SEQUENCE LISTING (111 sequences)

SEQ ID NO: 1 - DNA, 2600 bp, Rattus norvegicus
  misc_feature (1714)..(1714): n is a, c, g, or t
gcgatctactctcagtcttcctgagtcacaggctaccaaggacaagtagctctgtctgccctttgttgaggggagctgtgacactggtttgctgctgctgctgctgctgctgctgcatcttctttgggaaatcagactcaataaactgccatccatctgaggttctcaactcagagccatctacctggagcatgggactttcaaatgtccagctctggacaatactgctgtgggcattggcatgggtgcagagtacaagatctgcgtgcccgtcctgtggggccccaactctgacaccccaaggagaacgcgctctggtcctagagctagccaagcagcaaatcctggagggactgcacctaaccagccgtcccagaataactcgtcctctgccccaggcagcactgaccagagccctccggagactgcagcccaggagcatggtccctggcaaccgagagaaagtcatcagctttgctaccagcatagacaaatccacttcaacctaccgctccgtgctcaccttccaactgtcccctctttggtcccaccacctgtaccatgcccgcctctggctgcacgtgcctccctcttttccggccactctgtatctgaggatcttcggttgcggtaccacgaggtgcagaggatcccgcacgttcctagctgagcaccaaaccacttcctccggctggcacgccctgactctgccctctagcggcttgcggagtgaggaatctggagtcacaaaactccaactggaattcagacctctggaccttaacagcactactgccagactgccacggctgctgttggacacagcgggacagcagcgtcccttcttggaacttaagatccgagctaatgaacccggagcaggccgggccaggaggaggactcccacctgtgagtctgagacccccttatgttgtagacgagaccactatgtcgatttccaggagctggggtggagagactggatcctgcagccggagggataccagctgaattactgcagtgggcagtgcccgccccacctggctggcagccctggcattgctgcttccttccattctgctgtctttagcctcctcaaagccaacaacccttggcctgcgggttcttcctgctgtgtccccaccgcgcgaaggcctctctccctcctctaccttgaccataatggcaatgtggtcaagaccgatgtgccagacatggttgtagaggcctgtggctgcagctagcaagaggacctgaaggttttgagtgaagttcaaggttcaagttgggggttcccaagcaaaagggtcctgtgtctggaagcacaagttccggatccatactgacacccaacaagctgtgtagcagtatgcctgagtttgacccctgtggaactcaaatgggcatttttcttgtcccagattctggcctatttcaggctgtttcaaatgtggacagatgggtaaaacgtttcctttcgagaaactgcctggccagcaccatttcctacatcaagccctgttccaggacagcaggggatgccgtgggagggaaggaagaatgcagggaaaactacttagtctctcccgaaaaagaattcctcaagtaataaaggaggaattagaaggttaggcagattggggaaaggtatacaggctaagaacagggactgttgcctgctttgagcaaggtcaagaggaatatgagcaggcantgaggtgggagagcagttaggaggccctggaattgacagtcagtaaaaaggggtgctgaacccataagttctctagcttccccttggggagaggacccatgtaaatgacatacgtttatattttcttaataaaaaggagaaagaaaagcaacagtgtgtaaggggttgttaaaatgagccagaaacaaagtgtggtctggggacctctgtgcttcggggccttttcaaggggttttcaaactatttgcataatcacatagagaagttatttgtcatttactatcattattttttcattgttctgtgggaacagggtttacacttataactacagccctcaggaggctgagacaggaggatcgccgtgagttcaagaccagccttcatagtgaatgctgggctagcctgagctatggagtgagatcctgtctcaaagcaaaagaaacaaacaaaacaacaaagactaagtagtgagtctttccagagtgtattatatgatacagccgatgacattgtggcttatatattctcctttctctcctttaaacaattttatttatttttattgtatgtgtatgatgagtcttaggcctgcaccggcttgcagtgcccatagaggtcagaagaggacgctggatccttggaactaaagttagagcagtgtgagctgcctgtgtctgttaacagaactcaggtccacagtaagagaaacaaacaattaaaggctgagctacctctccacctgagacatgtgcacacacagtcacacattgttttgttttgtttggaaacagcccacgctatcttggaactctcaatcctcctgcctcagaccctcctgccctggaattaaaggcataaaccaccaaaaaaaaaaaaaaaaaaa

SEQ ID NO: 2 - DNA, 1731 bp, Rattus norvegicus
gtcgacagtctgggaggaatcgcccatcgagggaatagatgaaccacaccagagaacatggtagaagcggcccagcagagcaacgtgggctggggtgtacttcagtcggcagagtgctttgatctccagtagtggcccatgccatccagaggtggggagagagcttggggagcagactgtttggaaatggatggccctgtcttctttccatgtaactttccaactcccagttccattctccaccagcaacaacctcatgccatttgaggtgctacttcaatatcgctggcgtctactcatctatgtgaacttaagagtctggtgtcaggcgtgagctgaggtagaggtgggctcttctcagcctctataaaccaattacaccacttgagccaagcagtacacatgcactttctcctccgcctatcagcctagctcctgacaaggtttctctccagccttttactttcctggcttcaagaaaggcggatatataccagggtggggagttgcatttcagagtgagacggttctgtcttcacctaccacttgttggcgatgtgaccttgggcaaagctcattaacagcacagtgcctagttccctaatttgtaaaacatatgctataggtgtgacgattacgaagggctgacttttgtaatggctttgcttcagggatctgcagactcgttgagccacaattaggatgagaatcaaggtgcttcagacttgtgacaggcactggcggcccctcacatgatcctcagataccagattgtggcgtgtgctgctaggatcacttgtctttccagtctcccaacatctcttgggtccgtgatacgcgccccccaccccaagcccagcctgacgcggcggtggctcatgcgccctggagtcccgggctctagccacggaacacgtcccaactctggcgcccggctccgcccctagcctcgggcgcgtctctcccgccgcctgcttgggtgctggagcagccgcgcccgcggctctgggagctgattggctgtggactgcgggacgggcagccggagagcgcacacaccctcttcccgcagccaatgagcgcgcccacgtcactgtcttgggcggcccaaagagccggcagcccctcaataagccacattgttgcaccaactccagtgctagagtctcaggacaccacaggctacacggagttatcccgcttaggagacccgaaggcaggagcatcactccagtgactctgataaggtgcgatcgcccgagaggaacagaactgtcatttttgcgaagttgagccttacggatcccgtgggcgaagttagcgacgggacgctgagcaactagaccggtcggcaggagtgagacttaggtgccttctagtagttgtgacttaaaaaaaaaaaaaaaaaggaaaagaaaaaaggaggaaaacctgtttctggaaacgcgaggccctcagctggtgagccatcgtggttaagcttctttgtgtggctcctggagtctccgatcccagccggacacccgggcctggtttcaaagcggtcgaactgctctgcccgctccaccggtagcgctcgaacctcggtttctctactcgaccccgactcgccgcagcaggatgatcgcctcgcatatgatcgcctgcttattcacggagctcaaccaaaaccaagtgcagaaggtaagtcggcacgggcgggagctgctggctcgcttcgaccaagttgcg

SEQ ID NO: 3 - DNA, 4254 bp, Rattus norvegicus
gctcccatcttcgaggctcagctcaactcagagctacttcttccaaattctacatcttggcggacttcgcgaaggaaacccggagtgttacgtgaggtcctgatggagtttgaagagggccttaacggaagagcagacaagaacttctcaaagatgggcaaaaagagtaaaaaggagaaggagaagaaacctgctgttggcatattcgggatgtttcgctatgcagattggcttgacaagctgtgcatggctctgggaactctcgctgctatcatccacggaaccctgcttcccctcctgatgctggtgttcggatacatgacagatagttttaccccaagcagagacccgcattctgaccgagcgattactaatcaaagtgaaatcaacagtacacataccgtcagcgacacgagtctggaggaggacatggccatgtatgcctactattacacgggcattggtgccggtgtgctcatcgttgcctacatccaggtttcactttggtgcctggcagctgggagacaaatacacaagattaggcagaagtttttccatgccatcatgaatcaggagataggctggtttgacgtgaatgacgctggggagctcaacacccgtctcacagatgacgtctccaaaattaatgacggaattggtgacaaacttggaatgttctttcagtccataacgacattttcagccggttttataataggatttataagtggttggaagctaacccttgtaattttggccgtcagccctcttattgggttgtcatctgccatgtgggcaaaggtactgacttcatttactaataaggaactccaggcttatgcgaaagctggagcagttgccgaagaagtcttagcagccatcagaactgtgattgcgtttggaggacaaaagaaggaacttgaaaggtacaataaaaatttagaagaagctaaaagagttggcataaagaaagccatcacggccaacatttccataggtattgcctacctgttggtctatgcgtcttatgcactggcattctggtatgggacctccttggtcctctcaaatgaatattctattggacaagtgcttaccgtcttcttctctattttattggggactttcagtattggacatttagccccaaacatagaagcctttgcaaatgcaagaggggcagcctatgaaatcttcaagataattgataatgagccaagcatcgacagcttctcaaccaagggacacaaaccagacagtataatgggaaatttggaatttaaaaatgtttacttcaactacccatcacgaagtgaagttaagatcttgaagggcctcaacctgaaggtgaagagcgggcagacggtagccctggttggcaacagtggctgtgggaaaagcacaactgtccagctgctgcagaggctctacgaccccatagagggcgaggtcagtattgacggacaggacatcaggaccatcaatgtgaggtatctgcgggaaatcattggggtggtgagtcaggaacccgtgctgtttgccaccacgattgccgaaaacattcgctatggccgagaaaacgtcaccatggatgagatagagaaagctgtcaaggaagccaatgcctatgacttcatcatgaaactgccccacaaatttgacaccctggttggtgagagaggggcgcagctgagtgggggacagaaacagaggatcgccattgcccgggccctggtccgcaaccccaagatccttttgttggatgaggccacgtcagccttggacacagaaagcgaagccgtggttcaggccgctctggataaggctagagaaggccggaccaccattgtgatagctcaccgcttgtctacagtgcgcaatgctgacgtcattgctggttttgatggtggtgtcattgtggagcaaggaaatcatgaagagctcatgaaagagaagggcatttacttcaaacttgtcatgacacagactagaggaaatgaaattgaaccaggaaataatgcttatgaatcccaaagtgacactggtgcctctgagttgacttcagaagaatcaaaatctcctttaataaggagatcaattcgcagaagtatccacagaagacaagaccaggagagaagacttagttcgaaagaggatgtggatgaagatgtgcctatggtttccttttggcagatcctaaagctaaatattagtgaatggccctatttagttgtgggtgtactttgtgctgttataaatgggtgcatacaaccagtgtttgccatagtgttttcaaagattgtaggggttttttcaagagacgacgaccatgaaaccaaacaacggaattgtaacttgttttcccttctctttctggtcatgggaatgatttcttttgttacgtacttctttcaaggcttcacatttggcaaagctggagagatcctcaccaagcgactccgatacatggtcttcaaatccatgctgcgacaggatataagctggtttgatgaccataaaaacaccactggctcgctgactaccaggctcgctagtgacgcttctaatgttaaaggggctatgggctccaggcttgctgtagttacccagaatgtagcaaaccttggcacaggaattatcttatccttagtcttagtctatggctggcagcttacacttttacttgtagtaattataccactcattgtcttgggtggaattattgaaatgaaactgttgtctggtcaagccttgaaggacaagaaagagctagagatctctgggaagatcgctacagaagcaattgaaaacttccgcactgttgtctctttgactcgggagcagaagtttgaaactatgtatgcccagagcttgcagataccatacagaaatgctttgaagaaagcacacgtctttgggatcaccttcgccttcacccaggccatgatttatttttcctatgctgcttgtttccggttcggtgcctacttggtggcacgagaactcatgacgtttgaaaatgttatgttggtattttctgctgttgtctttggtgccatggcagcagggaataccagttcattcgctcctgactacgcgaaggccaaagtctcggcatcccacatcattgggatcattgagaaaatccccgagattgacagctacagcacggagggcttgaagcctaattggttagaaggaaatgtgaaatttaatggagtcaagttcaactatcccacccgacccaacatcccagtgcttcagggactgagcttcgaggtgaagaaggggcagacgctccgcctggtgggcagcagtggctgcgggaagagcacggtggtccagctgctcgagcgcttctacaaccccatggctggaacagtgtttctagatggcaaagaaataaagcaactcaatgtccagtgcgtccgcgcactgggcattgtgtcccaggagcccatcctgtttgactgcagcatcgccgagaacatcgcctacggagacaacagccgtgtcgtgtctcatgaggagatcgtgagggccgccagggaggccaacatccaccagttcatcgactcactgcctgagaaatacaacaccagagtgggagacaaagggactcagctgtcgggcgggcagaagcagcgcatcgccatcgcgcgcgccctcgtcagacagcctcacatcttacttctggatgaagcgacatcagctctggatacggagagtgaaaaggtcgtccaggaagcgctggacaaagccagggaaggccgcacctgcgttgtgatcgcgcaccgcctgtccaccatccagaacgcagacttgatcgtggtgattcagaacggccaggtcaaggagcacggcacccaccagcagctgctggcccagaaaggcatctatttctcgatggttcaggctggagcaaagcgctcatgagctgggagtatttgaggtgctaagtatttctaatattggtgttcaaacatggcacgtaaccaaagttaaaaggttaaaagcactgttaaaggtaatttcatcaagacgagaagccttcagagacttcataattaaatgaaccgaaattgaaaaaaaaatcattaaacagggccacattttttaattgtattatgtgattcaagagaacatatagtttttttaaaaagaaatgtgtagttttgtttcagtttttttaatttctaccctattcccttaaatgatcataaaggctgtaaaaagcactatttttttgcggcc

SEQ ID NO: 4 - DNA, 1016 bp, Rattus norvegicus
tgatgtttaaaattattggggctatcttaatgacggaaatagatgattgggaggggaagaggatgcctgattatatatatattcatgaaggtgtcgaaggtttataaagtcaatgtctgcagatgagaaagcagtggttctcttaggacttcttggggaagtgtggtcccagtggtcatcaccatgaacagccaagtgtcagcccggaaagcagggacactcctcctgctgatgatgtcaaaccttctgttctgccaaaatgtgcagaccctgccagtctgttctggtggcgactgccagacacctctcccggagctgtttgaccgtgtggtcatgctttctcactacatccataccctgtatacagatatgtttattgaatttgataaacagtatgtccaagatcgtgagtttattgccaaggccatcaatgactgccccacttcttccctagctactcctgaagacaaggaacaagcccagaaagtccctccggaagttcttttgaacctgatcctcagtttggtgcactcctggaatgaccctctgtttcaactaataactggactaggtggaatccatgaagctcctgatgctatcatatcaagagccaaagagattgaggaacaaaacaagcggcttcttgaagggattgaaaagataattagccaggcctatcctgaagccaaaggaaatgagatctacttggtttggtcacaactcccatccctgcaaggagttgatgaagaatccaaagacttggctttttataacaacattcggtgcctgcgcagggattcccacaaggttgacaattatctcaagttcctgaggtgccaaattgtccataaaaacaactgctaagcctacattcattccatgtacatctgagatgttcttaaaagtctatttcttcaaaggttctatttgcattacaactttcagcacatgcttaagtataattggtctcttcttaaataataaaaacaaacttttaaaaaaaaaaaaaaaaaaaa

SEQ ID NO: 5 - DNA, 1908 bp, Rattus norvegicus
gcgcggtgaagccagattaggatcagcgagcacttgaggatttagggccacaaaaaaccgcacaagatcgacagactatttctggagagctgcagaacgggcacgctggggtcgctggtgctggccatggtgatggaggtgggcatcctggacgccggggggctgcgcgcgctgctgcgagagcgcgccgctcagtgcctgcttctggattgtcgctccttcttcgccttcaacgccggccacatcgtgggctcagtgaacgtgcgcttcagcaccatcgtgcggcgccgcgccaagggcgccatgggcctggagcatatcgtgccgaacaccgaactgcgcggccgcctgctggccggagcctatacgcccgtagtgctgctggacgaacgcagcgccgccctggacggccgcaagcgcgacggcaccctggccctggccgcgggcgcgctctgccgagaagcgcgctccactcaagtcttcttcctccaaggaggatatgaagcgttttcggcttcctgccctgagctgtgcagcaaacagtccacccccatggggctcagcctcccgctgagtactagtgtgcctgacagtgcagaatccggatgcagctcctgtagcacccctctctacgaccaggggggcccagtggagatcctgtccttcctgtacctgggcagtgcttaccatgcttcccggaaagatatgctcgacgccttgggtatcactgctttgatcaacgtctcggccaattgtcctaaccactttgagggtcactaccagtacaagagcatccctgtggaggacaaccacaaggcagacattagctcctggttcaacgaggcgattgactttatagactccatcaaggatgctggaggaagggtgtttgtgcactgccaggccggcatctccaggtcagccaccatctgccttgcttacctcatgaggactaaccgagtgaagctggacgaggcctttgagttcgtgaagcagaggcggagtattatctcccccaacttcagcttcatgggccagctgctgcaatttgagtcccaagtactggcccctcactgttctgcagaagctgggagcccggccatggctgtccttgaccggggcacctctactacaacggtcttcaacttccctgtctccatccctgttcaccccacgaacagtgccctgaactaccttcaaagccccatcacaacctctccgagctgctgaagggccaggggaggtgtggagtttcacgtgccaccgggacgacactcctcccatgggaggagcaatgcaataactctgggagaggctcatgtgagctggtccttatttatttaacacccccccccccaaacacctcccgagttccactgagttcccaagcagtcataacaatgacttgaccgcaagacatttgctgaactcagcccgttcgggaccaatatattgtgggtacatcgagcccctctgacaaaacagggcagaagggaaaggactctgtttgagccagtttcttcccttgcctgttttttctagaaacttcgtgcttgacatacctaccagtattaaccattcccgatgacatacacgtttgagagttttaccttatttatttttgtgtgggtgggtggtctgccctcacaaatgtcattgtctactcatagaagaacgaaatacctcactttttgtgtttgcgtactgtactatcttgtaaatagacccagagcaggctttcagcactgatggacgaagccagtgttggtttgtttgtagcttttagctatcaacagttgtagtttgtttatttatgatctgaagtaatatatttcttcttctgtgaagacattttgttactgggatgactttttttatacaacagaataaattatgacgtttctattg

SEQ ID NO: 6 - DNA, 602 bp, Rattus sp.
acaggtggtgtccgatgggtacagcctttattgtttctccagcattttccagaagagtggtgtcattcgagggccataaaggatgggggagtaaaaaataacataaacaaactgaacagaaacccaggagggccgcgagaagggctgagatggggcatggggggtggagaggtgggagacccaagcagtcagcgccacttagctttcggccaccagagtggagaattcttcaaccccaatcttgccgtccccgtccttgtctccagcagccatcagcgtctttgtttccttagcagacaagtctctggcatctgaggagaagcccttcagaatggaccccagctcatcctcctcaatgaagccacttttgtctttgtccagaatgtggaacaccttcttcacatcatccgcactctttttcttcaggcccaccatctggaagaactttttgtggtcgaaggagtctgcagcagtaaaggctcctatcgccttcttgatgtcctcagcgctgagcaagtctgtcatcgacatcctgcaacttggatgaccagaagtcagagcctatatagaaaagctgggctggtgggagatcccctcgtgcc

SEQ ID NO: 7 - DNA, 1495 bp, Rattus norvegicus
caatccaagacaagatgtccagcaagggctctgtggttctggcctacagtggtggtctggacacctcctgcatcctcgtgtggctgaaggaacaaggctatgatgtcatcgcctacctggccaacattggccagaaggaagactttgaggaagccaggaagaaggcactgaagcttggggccaaaaaggtgttcattgaggatgtaagcaaggagtttgtggaagagttcatctggcctgctgtccagtccagtgcactctatgaggaccgctatctcctaggcacctctctcgccaggccttgcatagctcgcaaacaagtggaaattgcccagcgcgaaggggccaagtatgtgtctcacggcgccacggggaagggcaatgaccaggtccgctttgagctcacctgctactcgttagcaccccagattaaggtcatcgccccctggaggatgcccgagttttacaaccggttcaagggccgaaatgatttgatggaatacgcaaagcaacatggaatccccatccctgtcacccccaagagcccctggagcatggatgagaaccttatgcacatcagctacgaggctggaatcctggaaaaccccaagaaccaagcacctccaggtctctacacaaaaactcaggaccctgccaaagcacccaacaccccagatgtccttgagatagaattcaaaaaaggggtccctgtgaaggtgaccaacgtcaaagatggcactacccacagcacatccttggacctcttcatgtacctgaatgaagttgcgggcaagcatggagtagggcgcattgacatcgtggagaaccgcttcattggaatgaagtcccggggtatctacgagaccccagcagggaccatcctttaccacgctcatttagacatagaggccttcaccatggatcgggaagtacgcaaaatcaagcagggcctgggcctcaaattcgcagagctcgtatacaccggtttctggcacagccctgaatgtgaatttgttcgccactgcatcgacaagtcccaggaacgggtggaaggaaaggtgcaggtatctgtcttcaagggccaggtgtacatccttggccgggagtctccactttcactatacaatgaagagctggtgagcatgaacgtacagggtgactatgaacccattgatgccaccggcttcatcaatatcaactcgctcaggctgaaggagtaccatcgccttcagagcaaggtcaccgccaaatagaccgtgacaaagaggccgggcctccccgctctgcagctctcccaggctccagcattaattgttgtgataaatttgtaattgtagcttgttctcctaccacctgactggggctgctgtgccccccctcacctcccccccacccacaggctttgttccctggtcccctatagcctacaaaagtggtcatcgaagggaagggggggtggcaggcagctgcagaaagcgcgtaaaatgacaattaa

SEQ ID NO: 8 - DNA, 2559 bp, Rattus norvegicus
tggatccccaaattgctttttaaaaaatatcttggaaactttgtcctttgctgaattacgacactgtccacctttaatttcctcgaaaactccaatcactcggctgaagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaattactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgccccctcagtccgaggagatgatgccacacagcgggtcggtttactacaagccctcttcgcccccgacacccagcaccccgggcttccaggtgcagcatagcccgatgtgggacgatccgggctcccttcacaacttccaccagaactacgtggccactacgcatatgatcgagcagaggaagacacctgtctcccgcctttcactcttctcctttaagcagtccgccccgggcactcctgtgtctagctgccagatgcgctttgacgggcctctgcacgtccccatgaacccggagcccgcgggcagccaccacgtagtggatgggcagaccttcgccgtgcccaatcccattcgcaagccggcatccatgggcttcccgggcctgcagatcggccacgcgtcgcagttgcttgacacgcaggtgcccccgtcgccgtcccggggctctccctccaatgagggtctgtgcgctgtttgcggtgacaacgcggcctgtcagcattacggtgttcgcacttgtgagggctgcaaaggtttctttaagcgcacggtgcaaaaaaacgcgaaatatgtgtgtttagcaaataaaaattgcccagtggataagcgccgccgaaatcgttgtcagtactgtcggtttcagaagtgcctggctgttgggatggttaaagaagtggttcgcacggacagtttaaaaggccggagaggtcgtctaccctcaaaaccgaagagcccacaggatccctctcccccctcacctccgggatctgatcagtgccctcgtcagacccacgtcgactccaatccggcaatgaccagcctggactattccaggttccaggcaaaccctgactatcagatgagtggagatgatactcaacatatccagcagttctacgatctcctgactggctctatggagatcatcagagggtgggcagagaagattcctggctttgctgacctgcccaaagccagtcaggacctgctttttgaatcagctttcttagaattatttgttctacgcttagcatacaggtccaacccagtggagggtaaactcatcttttgcaatggggtggtcctgcacaggttgcaatgcgtgcgtggctttggggaatggattgattccattgttgaattctcctccaacttgcagaatatgaacatcgacatttctgccttctcctgcattgctgccctggctatggtcacagagagacacgggctcaaggaacccaagagagtggaagagctacaaaacaaaattgtaaattgtcttaaagaccatgtgactttcaataatgggggattgaaccgacccaactacctgtccaaactgttggggaagctcccagaacttcgcaccctttgcacacaggggctccagcgcattttctacctgaaattggaagacttggtaccaccaccagcaataattgacaaacttttcctggacaccttacctttctaagactttctcccatgcacgtcaaagaactggaaagaaaaaaaaaatccagagggggctggtcaagatgggtagagagctggctgaagtgtccggttcatgtctcccttctgtagacccctagccctcacccctaaagtaaacaaacaaacaagcaaacaaacaaacacaaattaaaaactgttgctatttcctaacctgcaggcagaacgtgaaagggcattttggctccggggcatcctggatttagaaaacggacaacatacacagtacagtggtataaacttttttattatcagttcaaaatcagtttattgttcagaaggaagattgcaaatgtatgatgggaaatgtttggccatgcttgcttgttgcagttaagacaaatgtaaggcaaatgtaacacacacacacacacacacacacacacacacacacacctcttaatgggaccctcatattttgccctttaacaagacttcaaagttttctgctgtaaagaaagctgtaatatatagtaaaactaaatgttgcgtgggtggcatgaattgaaggcagaggcttgtaaattttatccaatgcagtttggctttttaaattattttgtgcctatttatgaataaatattacaaattctaaaaagtaagtgtgtttgca

SEQ ID NO: 9 - DNA, 628 bp, Rattus norvegicus
atggctgcaataccagaagaaggctcttgtgtcaacttcaaagaaatgatgtttattgacaacacactttaccttatacctgaagataatggagacttggaatcagaccactttggcagacttcactgtacaaccgcagtaatgcggagcataaatgaccaagttctcttcgttgacaaaagaaacccgcctgtgttcgaggacatgcctgatatcgaccgaacagccaacgaatcccagaccagactgataatatatatgtacaaagatagtgaagtaagaggactggctgtgaccctatctgtgaaggatggaaggatgtctaccctctcctgtaaaaacaaaatcatttcctttgaggaaatgaatccacctgaaaatattgatgatataaaaagtgatctcatattctttcaaaaacgtgtgccaggacacaacaaaatggaatttgaatcttccctgtatgaaggacactttctagcttgccaaaaggaagatgatgctttcaaactcgttttgaagaggaaggatgaaaatggggataaatctgtaatgttcactcttactaacttacatcaaagttaggtattaaggtttctgtattccagaaagatgattagcacacatg

SEQ ID NO: 10 - DNA, 1067 bp, Rattus norvegicus
acggaggaccgtcagccagcaatcgcgtagctcgatcgatcacccaggaagagaaattgtaagcagataagaagaacgcgttcggtttgggacaccatttgcagctggaaatggggaatggactgtcagaccagacttccatcctatccagcctgccgtcctttcagtcctttcacattgttattctgggtttggactgtgctggaaagacaacagttttatacaggctgcagttcaacgaatttgtaaatactgtacctaccaaaggatttaacactgagaaaattaaggtaaccttgggcaattccaaaacagtcacttttcacttctgggatgtaggtggtcaagagaaattaagaccactgtggaagtcatatacccgatgcacagatggcattgtgtttgtggtggactctgttgatgttgagagaatggaagaagccaaaactgaacttcataaaataactaggatatcagaaaatcagggagtccctgtgcttattgttgctaacaaacaagacctgaggaactcactctctctctcagagatcgagaagttgttagcaatgggtgaactgagctcatcgactccttggcatttacagcccacctgtgcaatcataggagatgggctaaaggaaggacttgagaaactacatgatatgataattaaaagaagaaaaatgttgcggcaacagaaaaagaagagatgaatggcagtacttttatatcggtgtggaataggttttacttggtctgatttctgacaagctgaagagtgtctacagcctggtttgcctgtctgccctcacggatgctattaaagctttgttttgttgaacagtcagatacccaactctgttgccttgtggaagatgagtaaatgcaatgcttcttaaaggggtctcttctccgtgacccacaaatcttttggtactaccattttgggaagccaaaaaaggctagtaattgaccagaaaacaattttgtggaaatttgacctgaagttagtgaaataaaactttgaagagtgtaaaaaaaaaaaaaaaaaaaaaaaaaaaaaaaaa

SEQ ID NO: 11 - DNA, 2641 bp, Rattus norvegicus
gccctggaagccctgctttcctgacaccctacaggactcacaggcaggccatggcagctctggcagcgggagtatccaagcaacgtgcagttgctgaggggcttggctctaaccagaatgctgtgaagtatctgggccaggacttcgagaccctgaggaagcagtgcttgaactcgggggtcctatttaaggacccagaatttccagcatgtccatcagctttgggctacaaggatctaggaccaggctccccagacactcaaggcatcgtatggaagcgacccacggaattgtgtcccaaccctcagtttattgttggtggagccacacgcacagacatccgccaagggggtcttgtgtgggcacatgcgtctttgtgattctctgctggagaaacctgaacatgggctgaactgtaggactcccttgatggtccaagagactgctggcttctagcagccattgcctccctcaccttgaatgaaaagctgctttaccgggtgcttcccagggaccagagcttccagaaggactacgcgggcatatttcatttccagttctggcagtatggagagtgggtggaggtggtcattgatgaccggctccccaccaagaacgggcagctgctttttctacactccgaagaaggcaatgagttttggagcgctctgctggagaaagcctatgccaagctaaatggttcatatgaggctcttgttggaggctccacaatcgaggggtttgaggatttcacaggtggcatctctgagttttatgacttgaagaagcctccggaaaatctgtactacatcatccagaaggccctccgcaaaggctctctgctgggctgctccattgatgtctcaactgcagctgaagcagaagccaccaccaggcaaaagctggttaagggtcatgcatactctgttactggagtcgaagaggtgaatttccatggccgtccagagaagctgatcaggttgaggaacccgtggggtgaagtggagtggtcgggagcctggagtgataatgcacctgagtggaattacatagatccaaggaggaaggaggagctggacaagaaagcagaagatggcgagttctggatgtccttttcggatttcttgaagcagtactcccgactggagatctgcaacctgtccccagactctctgagcagtgaagagatacacaaatggaacctagtactcttcaacggccgctggacacggggttctacagccgggggctgcctgaactacccaggcacgtactggaccaacccccagtttaaaatccatttggatgaggtggatgaggaccaggaggagggcaccagtgaaccctgctgtaccgtgctgctgggtctgatgcagaagaatcgaagacgtcaaaagaggatcggccagggcatgctcagcataggctacgctgtctaccagattcccaaggaggttctcagcacgatcaggagtgtaaatagaccagcagggtccagattcatagacttgggcccaggctatgttcccagattgcagactactggtctgtcctgcccttggatcacagatgcacgccacttatcatggaggggacggcccagttggagagtcacacggatgcacaccttggccgggacttcttcctgggacgccagccctctacctgctccagcacttacatgaaccttcgggaggtgtccagcagggtccgactccccccgggacagtacctggtggtgccatccaccttcgagcccttcaaggatggtgacttctgcttgagggtgttctcagagaagaaggccaaggctctggaaattggggatactgtatctggacaccctcatgagccacatccccgtgacatggatgaagaagatgaacatgtccggagcctgtttgaggagtttgtgggcaaggattctgagatcagtgctaatcagctcaagagggtcctgaatgaagtactttctaaacgaacagacatgaaatttgatggattcaacatcaacacttgcagagaaatgatcagcctgctggatagtgatgggacaggaagcctgggacctatggagttcaagactctctggctgaagatccgcacatatctggagatcttccaagaaatggaccataaccatgtagggaccattgaagcccatgagatgaggacagctctcaagaaagcaggtttcaccctcaacaaccaggtgcagcagaccattgccatgaggtatgcgtgcagtaagcttggtgttgacttcaacggttttgtggcgtgtatgatccgcctggagaccctgttcaaactgttcaggcttttggataaggaccagaatggcattgtccagctctccctggctgagtggctgtgctgtgtgctggtctgacccgctgtttggacatcaacaacttccctgtctcccacttgtccctttcagtcttatgaacatgtgacctcaggtggcatttactgactgttgtattgttccagccaactgttgttcctgagacactgcctttcccaacagagcagtctagggagccccagatctctcagcagcaccgagctatgagctaactgggcagatcccagggttcagcagaaggaaaagaatcaattaaagttgtgggccagg

SEQ ID NO: 12 - DNA, 560 bp, Rattus sp.
aaatgaaacaaggtttatgccacacagtccaacaatgtataaagagcttgaagtagaaaagcttgtggaaaatgtatactctttacatggtacatacagttttatagctcataaataaacacagagaacaggagccattgttcacattaccgaccagcaacacagagcagcagtaacaacacaaacacaatttccttcgactcagccacaaaaacttgaaatggtctcaccatacttgaggaaaatgacaggtggtgaagtgttacagccacacccacatcaccaagtgctttaagttgtctctgggttagaaatatcaacaaccgtattaactccagagagaagaaggcagggtaagaccctgtcctataaggcacttaataaattgacactacccttgtcttaaaatactgcaaatttgccttgaaatatgctgatgttttacggcgaaatttaggtttcttttatccaggtttagacatagacataatttgaataaattgataatactgtctgatggccactctttacaccagcttcaaaatggacccatttaaact

SEQ ID NO: 13 - DNA, 1081 bp, Rattus norvegicus
gcttggaacggtacgcgaagaaaagtgaccatggacaacaacgaaacctccgtggattcaaaatccattaataattttgaaacaaagactatccatggaagcaagtcgatggactctggaatatatctggacagcagttacaaaatggattaccctgaaatgggcttgtgtataataattaataataagaacttccataaaagcactggaatgtcagctcgcaatggtaccgatgtcgatgcagctaacctcagagagacattcatggccctgaaatacgaagtcaggaataaaaatgaccttactcgtgaagaaattatggaattgatggatagtgtttctaaggaagatcacagcaaaaggagcagttttgtgtgtgtgattctaagtcatggagatgaaggagtaatttttggaacgaacggacctgtggacctgaaaaaactaactagtttcttcagaggcgactactgccggagtctgactggaaagccgaaactcttcatcattcaggcctgccgaggtacagagctggactgcggtattgagacagacagtggaactgacgatgatatggcatgccagaagataccagtgggggccgacttcctgtatgcttactctaccgcacccggttactattcctggagaaattcaagggacgggtcatggttcatccagtcactttgcgccatgctgaaactgtacgcgcacaagctggaattcatgcacatcctcactcgtgttaaccggaaggtggccatggaatttgagtccttctccctggacgccactttccacgcaaagaaacagatcccgtgtattgtgtcaatgctcacaaaagaactgtacttttatcactaaaggaatgactgggagtggggtaggggcatgtttctgttttggtttttttttggtttttggtttgttttttttttttttatttgaatgccaaatgagaaaactgtcagggagactttttttttcccctctcatttaaatcaaatccgatgttccaggtcgtcattgaacaataccactgcctgcaatgcagccacaatacaatacctcagctttgata

SEQ ID NO: 14 - DNA, 387 bp, Rattus sp.
gacatgtctcagaatttgctttattaacagtatcttgctctcacagctagatctttgggattggataggattcaatcagtaggtggtgtcggagctccttcttctggcttcattgtgataactttttgaagaaaacttcgaattcttcatagctaacttctccatcaccgttcttatccagctctttaaagagattgtctagagtacttgaagccttcaggaggctggggaactctgactgaatcagcagcttcagctcctccttggacagctggtttggatcgccttctttggctgcatatttttgaaaaatgctcttcatttcttcgggagatttcttagcgctcatttttctgtgctgcttgctgtcagtgagtgctgccagag

SEQ ID NO: 15 - DNA, 2668 bp, Rattus norvegicus
gagtggccaggagcaaagtctggaagccggcgagccacggagcagagcacacagagctatgaggcttgcacaaatgtgtctggccttcggctgggcagctgtcatattggttctacagacggtagacacggcatctgctgtgaggactccttatgacaaagcgagggtatttgcagacctgagcccccaggagataaaggctgttcacagcttcctgatgaacagggaggagctggggctgcagccgtccaaggaaccgactttggccaaaaactctgtgtttctcattgagatgctactgcccaagaagaagcatgtgttgaaatttctggatgaaggaagaaaaggtcctaaccgggaagctagggctgtcatcttcttcggtgcccaggactaccccaatgtcactgagtttgctgtggggcccctgccacggccctactatattcgagcactatcccccaggccagggcaccatctgtcctggtcatccaggcccatctccacagcagagtacgacctcctctaccacacgctgaagagagccaccatgcctctgcaccagtttttccttgacaccactggcttctcattcctaggctgtgacgaccgatgcttgacttttactgacgtagctccacgtggtgtggcgtctggtcagcgtagaagctggtttattgtgcagcgctatgtggaaggctatttcctgcatcctacagggctggagatcctattggatcatgggagcacagatgtccaggactggagagtggagcagctctggtataacggcaagttctacaacaacccagaggaactggctcggaaatacgcagttggagaagtggacacggtggtcctcgaggacccactgcccaatggcacagagaagcccccactcttttcttcctacaaaccccggggggaattccatacaccagtcaatgttgctggcccccacgttgtccagcccagcggcccccgatataaactagagggcaacactgtgctctatggaggttggagcttctcttatcggctaagatcctcttctgggctgcagatcttcaatgtgctctttggaggtgagcgtgttgcctacgaggtcagtgtgcaggaggctgtggcactgtatggaggacacacacctgcaggcatgcagactaagtacattgatgttggctggggcctgggcagtgtcactcacgagttggcccctggcattgactgtccagagactgctactttcctggatgccttccactattacgacagcgatggccctgtccattatccacatgctctgtgcctctttgagatgcccacaggggtgcccctgaggcgccactttaactcaaactttaaaggtggcttcaacttctatgcgggtttgaagggatatgtgctggtgctacggacgacatcaactgtctataattatgattacatctgggatttcatcttctactctaacggggtgatggaggccaagatgcacgccactggctatgtccatgcgaccttctacactcctgagggactgcgccatggcactcgcttacaaacccacctgcttggtaacatccacacccacctggtgcactaccgtgttgacatggacgtggcaggcaccaagaacagcttccagacactgacgatgaagctggaaaacctcaccaatccctggagcccaagtcactccctggtccagcccacactcgagcagacccagtactcccaggagcaccaggctgcattccgcttcggacagactctgcccaagtacctgctctttagcagcccccaaaagaactgctggggtcacaggcgcagctaccgcctgcagatccattctatggctgagcaggtgctgccaccaggctggcaggaggagcgggctgtcacttgggccaggtatcctttggctgtaacaaagtatcgggaatctgagcgctacagcagcagcctctacaaccagaatgacccctgggatccccccgtggtctttgaggagttccttcggaacaacgagaacattgaagatgaggatttggtggcctgggtgacagtgggcttcctgcacatccctcactcagaagatgtccccaacacagccacacctggaaactctgtgggcttcttgctccggcccttcaacttcttcccagaggacccatccctggcttctagagacactgtgatagtgtggccccaggacaagggcctcaaccgtgtccagcgctggatccctgaggacaggcgctgcttggtgtctcctcctttcagctataatgggacctacaagcctgtgtgatgggtcagccccagcctctgcagcacacccagagcctcacaaagacagggaaaacaaacaaacgaaacttctgtctctaccctgtatgctaccttttagctctactcgtgtttcattaccatacctgccacagacttccaagaccattgcaaaggaaggaccacaattttctgccggtggttcggtggtttggagggtggtgttttttaaatatttgaggtgtttatcttcctatcaggagactttagagaaattgtctcacctcagtgatgggagggagtaaggaaactgtcatatgaaaatctaataaaatgacacctttgctctttagacatcaaaaaaaaaaaaaaaaaa

SEQ ID NO: 16 - DNA, 1140 bp, Rattus norvegicus
ggatcctaacttcttcccaagcaccagtcactggagacataggagaggcaagaagcctctgggcaactgataaggattccccgcacccaggggaggcgatccccaagcccagcctctcagggtcctgacaagccatgctgcggagagcaatcacaggccagccaatgacagctgctccaagggcgtgtccttacctcctgcacatataaatgaaacttgctgcggggccaatacattctcattctgatagactcaggaagcaatcatggtgctctctgcagctgacaaaaccaacatcaagaactgctgggggaagattggtggccatggtggtgaatatggcgaggaggccctacagaggtaacatcaggaccctgttctttaaggacagcaggatccaaaccggaccagggactcagtgggtagctcctaagtgtgctttcccgtggcctcaacttatctctccttctcacaggatgttcgctgccttccccaccaccaagacctacttctctcacattgatgtaagccccggctctgcccaggtcaaggctcacggcaagaaggttgctgatgccctggccaaagctgcagaccacgtcgaagacctgcctggtgccctgtccactctgagcgacctgcatgcccacaaactgcgtgtggatcctgtcaacttcaaggtgtgagctcagacctggcagggggcacctgggaccttcaaggatcccttggggcagtggtgaggggcacagggggagggggaaggggttcctcatgcccagggcagggaacacagtgttccaggaaggggagcttacccagcagggtgtctactactagggactgacccttcctgtctctgcagttcctgagccactgcctgctggtgaccttggcttgccaccaccctggggatttcacacctgccatgcacgcctctctggacaaattccttgcctctgtgagcaccgtgctgacctccaagtaccgttaagccacctcctgtcgggcttgccttctgaccaggcccttcttccgtcccttgcacctgtctttgaataaagcaaaagtaggaagaatcccgtgtgcctgtttctcacatgtgcaaaggtgacaatgtttggtatgggaaaatcctcc

SEQ ID NO: 17 - DNA, 626 bp, Rattus sp.
gtcttataaaattgttttattttattctatgtaaacttctttctcaggaatggtaccaaagtgacttgcttacagggatttataatttgggataaacataattaacaaaacaagtatttctctattgtacagatgaatgatctaaaacatatttataggctacattattcaaagaaggtaattagacttactgacaaagcaactttcctacttaagccttgcaataggaagaaaacaacggctaagagatgttctcctcggaatcttacagagcagttgggatgtttagtttcctccacacagagccaccaggattttttcatagtctcctttggtttcatccaggatggcttggcagagagggattccgtacttcttctggtaaaatactttgatttcattcatgtcaatttccgaacgggagaccataatcctgatcaatgtcttatggcgagttccagcacccttcatggcttcatacagtttctcagcaaagaaagctggagtgctggtggcacacttcacaatggttgtgaggcacttctcaatgtcacccttcagttccagatccaaggctttgttcatgtcatgttgactgtactttctataattctgaaacactttcc

SEQ ID NO: 18 - DNA, 508 bp, Rattus sp.
gccattaaaaaactttattttgatttttttttttttacaaaggatagccccactttggggtgaaaatatatttggttaagtacaaaatatagtttttaatcatacaaaaagatacacaaaatacaaaaacaacaaagacgacacctccatcagctcacactttctggctgcacagttctcgggtggagtgcttagagtggagcttggagctctgacacaggacacggaacaaagtgctaaggcggagtaagttttggagtgctagaggccacggcgagagcagaactttctcctaacactggacaggaaatgggtctgggagcttccatggaaacctgcagttctcactgtgcctccatcactgttagggcatctcaatgtaagagtgtcaccagggtccagctgtcagcagcaacccttcatatgagtagtgtcagaatcacagaagtctgtgtgttgggggaaagcctccaaaagtaataaccaagctgggcctttaacctcagca

SEQ ID NO: 19 - DNA, 2642 bp, Rattus norvegicus
agtgtgctctaaaggcgacgggtttgtcggagcaccataatatggtgtgggaagtgaagacgaaccagaggcctcatgcagtacagagactcctgctggtgatggacgagagagctacaggagtgagtgactcattggagttgctgcagtgtaatgagaatgtgccgtcctctccaggatacaactcctgtgatgagcacatggagcttgatgaccttcctgaacttcaggctgttcaaagtgaccctacccaatctgccatataccagctcagttcagatgtatctcatcaagaatatccaagaccatcttggagccagaatacctcagacataccggaaaatactcaccgtgaagatgaggtggactggctaacagagttagcaaatattgccactagtccacaaagtccactaatgcagtgctcattttataacagatcatctcctgtacacatcatagctactagcaacagtttacattcctatgcacgcccaccaccagtgtcctctgcaaagagcggaccagccttccctcatgaccactggaaggaagaaacaccagtaagacatgaaagggcaaacagtgagtcagagtcaggaattttctgcatgtcctccctgtcagatgatgatgatctgggctggtgcaattcctggccatcaaccgtttggcattgctttctcaaaggcacacgactgtgcttccataaggaaagcaaaaaggagtggcaagatgttgaagactttgctagagccgccacctgtgatgaggaggagatccaaatgggcactcacaagggctatgggtctgatggtctaaagttgttatcccatgaagagagtgtgtcattcggtgagtctgtactgaagttgacatttgatcctggtacagtagaagatggcttgcttactgtggagtgcaagctggaccaccctttctatgttaaaaataaaggctggtcatcattttatccaagcttgactgtggtacagcacggcattccatgctgtgagattcatattggtgacgtatgtctacctcctggacaccctgatgccattaatttcgatgattcaggtgtttttgatacatttaaaagctatgacttcacacctatggactcttccgcagtctacgttttgagtagtatggctcgccagcgccgcgcatctttgtcttgtggaggacctggtactggtcaagagtttgcaggatctgaattcagtaaaagctgtggttcaccaggatcatcacagctctcctctagttctttatacactaaagctgtcaaaagccacagctcagggactgtgagtgccacttctcctaataagtgcaaaagaccaatgaatgccttcatgctttttgccaaaaaatacagagttgaatatactcagatgtatccagggaaagataacagagccataagtgtgatccttggtgacaggtggaaaaaaatgaagaacgaggaaaggaggatgtatacattagaagcaaaggctttggctgaagaacaaaagcgtttaaatcccgactgctggaaaaggaaaagaaccaattcaggctctcaacagcactaaacctgatgcttctgtctgtaagtctgtaccttgcatcagtactgactctgtacctgcataaggactgactctggacggaaaagcttggaagaccttggtctcaccatttgtcctacatttgtgtgaccataaaatgctgggagcattgagttttgtaatgatttaatgggtgaggaggatctgctttctccattagagcattaagtaagctaaaactatcaacattgtaaaccaaattgccttattttccttccaaacttcatatatgtctatcaggtaatataggcttgaaaattgatatcctgtggtgctaaagtacagtagaaagagagatgtgtatacatgtcttattttaaattgtacgaaggggaatttaaaaatatgtaactgctgtttatacatcagctccttactgcttattaatctgtattgtacccacgatatcaggaatgaagtagaagctggcctttccatgaatagccaccacatggcccagtgtctctgccaaacacagttgcttctagtctggccagtgccaagagacaacataacatggcaggtggatggtgtcattgtctcagctgagagccacctgcttcagataccatgggcgtgtgctgagtggatgcaccagggggcagcacagctccatgattcccagtgggggccaagccaggctaaggtgcagtcagttagcattgggacagtcattgtaatggtgctgttatcagttgaaaccatgctccctgtctcctcagccacatcccctccgtctctgctgtcatcttctctgtggtggggcagactttgcacttactgcagtgcaacacttgcactttacccctccggcttctacagtagagcaagtccttcggcagccctgctgctttcgctctgcgctacaatcatggcttctcatgttacttaccaagtggtatttttggttaggaatcacagctgtaaagttgatttcagttcattacacttcttaaacatattgcccctcaattttgcacactatattcttgtatattatttcaaataaaatgaaaaaagtct

SEQ ID NO: 20 - DNA, 1671 bp, Rattus norvegicus
cctttctttgcgagaagtgcaaaattggctagagccagagtctcgcccacctggtacagaaggagcgcgcgcctgtgtcccttggggacccacagttgcaaagagacagctgcttgatttggtcacccactcgcccagactataggagcctcccgggacactcttgagttgcacctttctgcagagcagactgtttgagcccccgcagtgctcgtaggatttgaagcgttctgatactggaggcttgctcgccgcgctcgcccgactctgggacacatttgtccgagccacaggagtgtgcgggtgactgacatatcatctcttcgactctccatgagctcatgaacctgggagcaggtgcccggaaatggcgcggcctctgagcgacaggaccccgggccccctgctgctgggtggcccggctggggccccccctggcgggggagcgctgcttgggctgaggagccttctgcagggaaacagcaagcccaaagaaccggccagctgtctcctgaaggaaaaggagcgcaaggcaactctgccctcagcccccgtcccgggacccgtcctggaaacggcgggcccagcggatgccccaactggggcggttagtggcggtgggtcccctcgggggcgctcagggcctgtggctggcccgagtctttttgcgccgctgctgtgggaacgcactttgcctttcggggacgtggaatacgtggacctggacgccttcttgctggagcacgggctaccgccgagcccgccgccccccgggggcctgtcgccggcaccctctccagcgcgcactcccgcgccctcccccgggcccggctcttgcagctcctcttccccccgctcctcgcccgggcacgcccccgcgcgggccactctgggagccgccggcggccaccgcgcaggcttgacctctagggacacacccagtcctgtggacccagacaccgtggaggtgctaatgacctttgaacctgatccggctgatcttgccctgtcaagcattccaggccatgagacttttgaccctcggaggcaccgcttctcagaggaggaattgaagcctcagccaatcatgaagaaggcaaggaaagtccaggtgcccgaggaacagaaggatgagaagtactggagccggaggtacaagaacaatgaagcagccaagaggtcgagagacgcaagaagactcaaggagaaccagatatctgtgcgggcagccttcctggagaaggaaaacgccctattgcggcaggaggtggtggctgtgcggcaggagctgtcccactaccgtgctgtgctttcacgctaccaggcccaacacgggacactgtgaggccacttccaccctgccagggcagagtcctgttccttgttcagacttacacctgacaccccccttcctccttgtcccatggccaatggtctggccagctaggtgcccaggaacgtcatgatgcagacaaatacatttatatttttaagaaaaagctagccttcccccacctcccttgtgggggtggggagggtcctgtgtgcacttttagcatgtcgaggaacccatccatccaaccgcctccatcaacacaatcctgaataaatcttgagaacccaaaaaaaaaaaaaaaaaaaaaaaaaaaaaaaaaaaaaaaaaaagaattc

SEQ ID NO: 21 - DNA, 2249 bp, Rattus rattus
aaaggtccaagcttaatggatgtatactcgtgactgagctctcggcccacaatcagccaaccgagggcttcatggatccgccaagatcatcaacacccagggactgggaccagatgaaggagggagaaggcagcgaggagaagttgaggaggaggatgaagatgaggaggaagaggatgaggaggaggaggaagaggatgaggaggaggaggacttgtcttctcctcaagggctacctgagcctctggagaatgtggaagtcccctctggaccccaggtcctcacagatggcccccgggaacatagcaagagtgctagcctcctatttggcatgcgaaacagtgcagccagtgacgaggactcaagctgggccaccttatcgcagggcagcccctcctatggctctccggaggacacagattccttctggaaccccaacgctttcgagacggattccgatctaccggctggatggatgagggtccaagacacctcagggacctactactggcacatcccaacagggaccacccagtgggaacccccaggccgggcctccccatcacaggggaacagtccccaagaagagtcccagctcacctggactggctttgctcaccaagaaggctttgaggaaggagagttttggaaggatgaacctagtgaagaggccccaatggagttgggactgaaggaccctgaggaggggacattgcccttctcagctcagagcctcagcccagagccagtgccccaggaggaggagaatctgccccaacggaacgccaatccagggatcaagtgtttcgctgtgcgctccctaggctgggtagagatgactgaggaggagctggccccaggacgcagcagtgtggcagtcaacaattgtatccgccagctctcctaccacaaaaacaatctacatgatccgatgtctggaggctggggagagggaaaggatctgctgctccagctggaggatgagacgctaaagttggtggagccacagaaccagacactgctgcatgcccagcccatcgtcagcattcgcgtgtggggcgttgggcgggacagtggaagagagagggactttgcctacgtagctcgagataagctgacccagatgctcaagtgccacgtgtttcgctgtgaggcacctgccaagaacatcgccaccagcctgcatgagatctgctccaagatcatgtctgaacggcgcaatgctcgctgcttggtaaatggactctccctggaccactctaaacttgtggatgtccctttccaagtggaattcccagcaccaaagaatgaactggtgcagaagtttcaagtctattacctagggaacgtgcctgtggctaaacccgttggggtagatgtgattaatggagccctggagtcagtcctgtcttccagtagtcgtgagcagtggaccccaagtcacgtcagcgtggcccctgccaccctcaccatcttgcaccagcagacagaagcagtgctgggggagtgtcgagtgcggttcctctccttcctggctgtgggcagagatgtgcacacgttcgcattcatcatggctgccggcccagcctccttctgctgtcacatgttttggtgcgagcccaatgctgccagtctctcggaggctgtgcaggctgcatgcatgctccgctaccagaagtgtctggatgctcgttcccagacctccacctcctgcctcccagcaccccctgcggagtcagttgcgagacgtgtagggtggactgtccgcaggggtgttcagtctctgtggggttccctcaagcccaaacgtctaggatcccagaccccatgaagagctcccatcctccctctacctgcttgttttgggccccagggaactcaagggtttggggcaggtaggggctgtggatgttcttcctacaccccatgtcccccgggctgcccctcctcagtagctggggttccctacctaggggctgggagagagaaattgaattccttcacagaagtcattacactggactgatgacatcggggccgagaagcaaagccagccttgggctctccatccccagtgtttgaggtggagcaggaggaactggtccaagccaggccccatcctcaaaggcccaataagggcaggaggggactggtctcggcatggatcccctgtcccctgttctgcggacacctccactgtactgatatcactaataaagtctgtctgtcgctgaaaaaaaaaaaaaaaa

SEQ ID NO: 22 - DNA, 3113 bp, Rattus norvegicus
cctagatgccccagtcttctactgctctgaccccacccgctttctcccggctcggtacagctggtgccggggggtgctgctttctgccctgcgctgcgcaccgttagtgccctgcccctgtccttccgatctcagagtctgcggagtgcccctatcgccgtccacctgtttcctcagaaaaaaggccagctcgtgatccctgctgcgttcctggggccatggcgggtctgggtcctggcgggggcgactcagaagggggaccccgacccctgttttgcagaaagggggcgctgaggcagaaggtggtccacgaggtgaagagccacaagttcaccgctcgtttcttcaagcagccaaccttctgcagtcactgtaccgacttcatctggggcattggaaagcagggcctgcaatgtcaagtctgcagctttgtggttcaccgccgatgccacgaatttgtgaccttcgagtgtccaggagctggaaagggcccccagacggacgaccctcgcaacaagcacaagttccgtctgcacagctacagcagtcccaccttctgcgaccactgtggttccctcctctacgggctggtgcaccagggcatgaaatgttcctgttgcgaaatgaatgtgcaccgacgctgtgtgcgcagcgtgccctccctttgcggcgtggaccatacagagcgccgtggacgtctgcaactggaaatccgggctcccacatcagatgagatccatattactgtgggtgaggcccggaacctcattcctatggaccccaatggcctgtctgatccctatgtgaaactgaagctcatcccggaccctcggaacctgacaaaacagaagacaaagaccgtgaaagccacactgaatcccgtgtggaacgagaccttcgtgttcaacctgaagccgggggatgtggagcgccggctcagtgtggaggtgtgggattgggataggacatcccgaaatgacttcatgggtgccatgtcctttggtgtctcagagctactcaaggctcctgtggatggatggtacaagttactgaaccaggaggagggcgagtattacaatgtaccggtggccgatgctgacaactgcagcctcctccagaagtttgaggcctgtaattaccccttggaattgtatgagagagtgcggatgggcccctcttcctctcccattccttctccatcccccagtcccacggactccaagagatgcttcttcggtgccagcccaggacgcctgcatatctctgacttcagcttcctcatggttctagggaaaggcagttttgggaaggtgatgctggcagagcgcagaggatccgatgaactctatgccatcaagatactgaaaaaagacgtcattgtccaggatgatgatgtagactgcacccttgtggagaagcgtgtgctggcattgggaggccgaggtcctggaggccggccacactttctcacacaacttcattccacctttcagactccggaccgcctgtattttgtgatggagtacgtcactgggggcgatttaatgtaccacattcagcaactgggcaagtttaaggagccccacgcagcattctatgccgcggaaatcgccataggcctcttcttccttcacaaccagggcatcatctacagggacctcaagttggataatgtgatgctggatgctgaaggacacatcaagatcacagacttcggcatgtgtaaagagaatgtcttccctgggtccacaacccgcaccttctgtgggaccccagactacatagcacctgagatcattgcctatcagccctatgggaagtctgtcgactggtggtcctttggagtcctgctgtatgagatgttggcaggacagccaccctttgatggggaagatgaggaggagctgtttcaagccatcatggaacaaactgtcacctatcccaagtcactttcccgggaagctgtggccatctgcaaggggttcctgaccaagcacccaggaaagcgcctgggctcagggccagatggggaacccaccatccgggctcatggctttttccgttggatcgattgggagaggttggagagactggaaattgcgcctccttttagaccacgtccgtgtggccgcagcggcgaaaactttgacaagttcttcacgcgggcagcgccagccttgaccccgccagaccgcttggtcctagccagcatcgaccaagctgatttccagggctttacttatgtgaacccggacttcgtgcacccagatgcccgcagccccacaagccctgtgcctgtgcccgtcatgtaatctcatctgctgccgctaggtgttcccagtgctccctccgccaagttggctgtaactcccatccacccccatccccgcctctagtccgaattttaggtctcttaaaccacccaaccttctggcctctttcacgcgccccaagtgggttctagacgctgttccccagcattgctggcattttaaacttcaaacagtctctagggcctttctgtgttctaggttcgttgtgctgagccctggtttttccccacccccaacatctggatgctgttccaactcttcccagaaaccccactccgtgtggggttctagactctatcttggtagttttatgccttctctctccctagaccacgttgggagaaatagtctcatgagattgcctgctccagactaagattccagatcagctctctgcatccttcaaggcccctcctacctccacttcagttgtagaattaagtgggaggctgggctccgtgttccaggccacctcccttccatgttctggggattcctggcatgcacggaggattctctccccgacttttctcagtcagcttttgttctagatttgttccagaacccttcactgctcacctgccccgtgcatggctccagccttggtcggaatcacacacacacacacacacacacacacacacacacacacacacacacacacaccccttgtcctccgcagtgcctgccactttctgggactttctcatcccccacgcccttcctttatcctctcccacccagacacagctgctggagaataaatttggagctctcgag

SEQ ID NO: 23 - DNA, 3611 bp, Rattus norvegicus
cccgcgggggaagacgcacgggcgggctcggctctcccggggagcggcccgggactgcaccgggaccggcgcctccccgctccggctgccctcggcctcgccccgggcccgggcggatgagccgggggcccggggggacatggaagcgctgacgctgtggcttcttccctggatatgccagtgcgttacggtgcgggccgactccatcatccacatcggtgccatcttcgaggagaacgcagccaaggacgacagggtgttccagttggctgtatcagacttgagcctcaatgatgacatcctgcagagtgagaagatcacctactccatcaaggtcatcgaggccaataatccgttccaggcggtgcaggaagcctgtgacctcatgacccaggggattttggccttggtcacgtccacgggctgtgcatctgccaacgccctgcagtccctcacagacgccatgcacatcccacacctctttgtccagcgcaaccccgggggttcaccacgtactgcctgccacctgaaccctagccccgacggcgaggcctacacactggcttcgagaccgcccgtccgtctcaatgatgtcatgctcaggctggtgacagaactgcgctggcagaagttcgtcatgttctatgacagcgagtatgatatccgtgggctccaaagctttctggatcaggcctcacggctgggtcttgacgtctctttacaaaaggtggacaagaacatcagtcacgtgttcaccagcctcttcaccaccatgaagacggaggagctgaatcgctaccgggacacactgcgccgggccatcctcctgcttagcccacagggggcgcactcgttcatcaacgaggctgtggagaccaacttggcttccaaggacagccactgggtcttcgtgaatgaggaaatcagtgaccccgagatcctggatctggtccacagtgcccttggcaggatgaccgtggtccggcaaatcttcccatctgcaaaggacaaccagaaatgcatgaggaataaccaccgcatctcttccctgctctgtgatccacaggaaggctacctccaaatgctgcagatctccaatctctatctgtacgacagtgttctgatgctggccaacgccttccacaggaagctggaagaccggaagtggcatagtatggcaagccttaactgcatacggaaatctaccaagccatggaatggagggagatccatgctagacaccattaaaaagggacacatcaccggcctcacaggagttatggagtttcgggaagacagctcaaatccctatgtccagtttgaaatccttggcacaacctatagtgagacttttggcaaagacatgcgcaagctggcgacctgggactcagagaagggcctgaatggcagtctgcaggagagacccatgggcagccgcctccaaggactgactctcaaagtggtgactgtcttggaagagccttttgtgatggtagctgagaatatccttggacagcccaagcgttacaaagggttctccatagatgtgctggatgcattggctaaagctctcggattcaaatacgagatataccaggcccctgatggcaggtatggccaccaactccataacacttcctggaacgggatgatcggggagctcattagcaagagagcagacttggccatctctgctattaccatcaccccggagagagagagcgttgtggacttcagcaagcgatacatggactactcagtggggatcctcatcaagaagccggaggagaaaatcagcatcttctcccttttcgccccctttgactttgcggtgtgggcctgcattgctgcagccattcccgtggtgggtgtgctcatattcgtgttgaatcggatacaggctgtaaggtctcagagtgccacccagcctcggccctcagcttctgcgactttgcacagtgccatctggatcgtctatggagcctttgtccagcaaggtggtgagtcttcggtgaactctgtggccatgcgcatcgtgatgggcagctggtggctcttcacgctcattgtatgttcctcctacacagccaaccttgctgctttcctcacagtgtccaggatggacagccccgtaagaacatttcaggacctgtccaagcaactggagatgtcttatggcactgtccgggactctgctgtctatgagtacttcagagccaaggggaccaatcccctggagcaggatagcacttttgctgagctctggcggaccataagcaagaatggaggggctgacaactgtgtgtccaatccttcagaaggtatcaggaaggcaaagaaggggaactacgcctttctgtgggatgtggccgtggttgagtatgcagccctgacagatgacgactgctcagtgactgtcatcggcaacagcatcagcagcaagggctatgggattgccctgcaacatggcagcccctacagggacctcttctcccagaggatcctggagctgcaagacacaggggacctggatgtgctcaagcagaagtggtggccacacacaggccgctgcgacctcaccagccattccagtgcacagactgatggtaaatcccttaagctgcacagcttcgctggggtcttctgcattttggccattggcctccttctcgcctgcctcgtggctgctctagagttgtggtggaacagcaaccggtgccaccaggagacccccaaagaggacaaagaagtgaatctggaacaggtgcaccggcgcatcaacagcctcatggatgaagacattgctcacaagcaaatttccccagcatccattgagctttctgccctggagatggggggcctggctccaagccaagctttggagcccacgagggagtaccagaacacccagctctcagtcagcacctttctgcctgagcagagcagccatggcaccagccggacactgtcgtcagggcccagcagcaacctgccactgccgctgagcagctcagccaccatgccctcaattcagtgcaaacacaggtcgcccaatgggggactgtttcgacagagtccggtgaagacccccatccctatgtctttccagcccgtgcctggaggcgtccttccagaggccctggacacctctcatggcacctccatctgactgtgtcgcctgccctcctgcccgccacccagcccacccgaccagcagagctttttaatacaagaaaatgacaacacagaccacacacacacacacacacacacacacacacacacacagactctttcatttttcttgtacatacgtgtaaataatgacagaatggagtggggtaaaagtgtattttgaatattcccaattttcgaagtcagtaaaaaaaaacaaaacaaaacaaaaactgtatgaatgactttgtaaattttgttctatatgaataaaaaggcaaattacttgtgatctattttatttctgtgagtggggtaggcaggccagtacagaggggattgatctgcaggtgctggtcctgcagggtccaagtggctcccccttcctggttgcatgaccctgtgccaggatgccgggacatcggtttgcaggtccg

SEQ ID NO: 24 - DNA, 4422 bp, Rattus norvegicus
gatcctgggagagtccgacgttcgccggctaacgcaggacaggtcactgaggtccgccaggtgtgtccgccgtttgtttcctgcccctcgccaggttcggtgtccgctgccgaggcccgaggcccttcttccgctgtcggcgcgcccccctgtctcgtttcggccccgccgggacaacgcctggagtcgcagcagccgggtgatcgcgcccgtcatcctctaccaacgtttccaggctgccctgctcgggagtgggtgagaatgaagctaaaggagatagaccgcacagcgatgcaggcgtggagccctgcccagaaccaccccatttacctggccacagggacatctgctcagcagttggacgcgacattcagtaccaacgcttcccttgagatattcgagctcgacctttccgacccgtccttggatatgaagtcctgtgctactttctcctcgtctcacaggtaccacaagttgatttggggacctcataaaatggattccaaaggagatgtttctggagttttgattgcaggtggagaaaatggaaacattattctctacgatccttctaaaattatagctggagacaaggaagttgtgattgcccaaaaggacaagcatactgggccagtgagagccttggatgtaaacatcttccagaccaatttggtggcctctggtgctaatgaatctgaaatctacatttgggatctaaacaattttgcaactccaatgacgccaggagctaaaacacagccaccagaagatatcagctgcatagcctggaacagacaggtccagcatatcttagcatcagccagtcccagtggccgggcaacggtctgggatctcaggaaaaacgagcctattatcaaagtcagcaaccacagtaactggatgcactgctctgggctggcatggcaccctgatgtcgctactcagatggtccttgcttctgaggatgatcgattaccagtggtccagatgtgggatcttcgatttgcttcctctcccctccgggttctggaaaatcatgccagggggattttggcaattgcttggagcatggcagatcctgaattgttgttaagctgtggaaaagatgctaagattctctgctctaaccctaacacgggagaggtattatatgaacttcccaccaacacacagtggtgctttgatatccagtggtgtcctcggaatcctgccgtcctgtctgccgcttccttcgatggtcgcatccgtgtctactccatcatgggagggagcatagacggcttaaggcagaaacaagttgacaagctttcgtcgtcttttgggaatcttgatccgtttggcacaggacagccgctccctccattacaaatcccacagcagacttcacagcacagcatcgtgctgcccctgaagaagccgcccaagtggatccggagacctgtgggcgcctccttctcattcggagggaagttggtgactttcgagaacgttacagggcagcctcagcagggagctgagcagccacggcggcagcccgtgttcatcagtcaggtggtgacagaaaaggacttcctcagccgctcagagcagctacagcacgtcgtcgagtctcagggctttatcagttactgccagaaaaagatcgatgcgtcccagactgacttcgagaagaatgtgtggtccttcttgaaggtaaagtttgaggaggattctcgtgggaaataccttgagcttcttggatatagaagagaagatctgggagagaagattgctttggccttgaacagagtggacggatccgatgtggctcttaaggactctgaccgcgtagcgcagagtgatggggaggagagccctgccgaggaagggcagctcctgggagagcgcattaaagaggaaaaacaggagtgtgactttctgccctcggctggcggtgggacctttaacatctccgtcagtggggatattgatggtttaattacccgggctttgctgaccggcaattttgagagcgcagttgacctttgtttacacgataaccgaatggctgatgccatcatattagccatcgcaggtgggcaggagctcttggctcaaacgcagaagaaatactttgcaaaatcccaaagcaagattaccaggcttatcactgcggtggtgatgaagaactggaaggagattgttgaatcctgtgatcttaaaaattggagagaggctttagctgcggtcctcacttatgctaagccagatgagttctccgccctgtgtgatctcttgggagccaggcttgaaagtgaaggagacagcctcctgcggactcaggcctgcctctgctatatctgtgccgggaatgtagagagattagttgcttgttggaccaaagctcaagatggtagcaaccccttgtctctccaggatctgattgagaaagttgtcatcctccgaaaagctgtacagctcacgcaagccctggacacgaacactgtcggggctcttctggccgagaagatgagtcagtatgccaacttgttggcagcccagggcagcattgccgcggccttggcgtttctgcctgacaacaccaaccagccagatatcgtgcagctgcgtgacagactctgcagagctcaaggcaggtctgtgccggggcaggagtcctctaggagctcctacgaggggcagccgctgcccaagggcgggcccgggccgtttgctggtcacccgcaggtgtccagagttcaaagtcagcaatattatccccaggttagaattgcccctactgtcactacctggagtgacagaactcccactgccctccccagccatccacctgcagcctgtccctctgacacacagggagggaatcctccacctccaggtttcataatgcatggcaacgttgttccaaatagccctgccccactgccgacgtctccaggccacatgcacagccagccaccaccttatccgcagccgcagccttaccagccagcccagcagtattccttgggaacaggggggtcagcagtttatcgacctcaacagcctgttgctcctcctgcttctaagcgttaccctaacgccccatacgtatctcctgtcgcttcctactccgggcagcctcatatgtacacagcacagccagcctcctcccctacctccagctctgctcctcttccccctcccccttcctctggagcgtccttccagcatggcggaccaggagctccaccgtcatcttcagcttatgcactgcctcctggaacaacaggtacaccgcctgctgccagtgagctgcctgcgtcccagagaacaggtcctcagaatggttggaatgatcctcccgctttgaacagagtaccgaagaagaagaagctgcctgagaacttcatgcctcctgtccccattacgtcaccaatcatgaaccctgggggagatccccagccacaggggctgcagcaacagccttcagcttcagggccacggtcaagccacgcctccttcccgcagccgcacctagccggcggccagcccttccacgggatccagcagcctcttgctcaaacaggcatgccaccgtctttctcgaagcccaacactgaaggggcccccggcgctcccattgggaacaccatccagcatgtgcaggctctgccaacagaaaaaattaccaagaagcctattccagacgagcacctcattctaaagactacgtttgaggaccttatccagcgctgcctctcctcagctacagacccccaaaccaagaggaagctggacgacgccagcaaacgcctggagtgtctgtacgacaaacttagggaccagacactttctccaacaatcatcagcggtctccacagcattgcgaggagcattgagacgaggaactactctgaagggttgaccgtccacactcacatcgtcagcaccagcaacttcagtgagacctcagccttcatgccagtcctcaaggttgtgctcagccaggccagtaagctgggcgtctgagacagctgccatgacctccgactccgccacttctccaaagaaagccatgctaagactgggaccagcccctcattagcatgtttccatggcacccgccacccaggagctttgacatttctgcaggcaaactcccctagaactgcttgaaccccccctcccaccccgcccgtgctttcctttgttttaatcccctcgcccattatgattttcccattctgcaaatagtgtctttcctggattacacatagtgtggtttcctgagggattctgacaaaatgtggtttttaaaacttgggtgtactttttaggaaaattgcatctggctgtgtataaaacagacctaaaatagatttctttaatgtcttccctaccttcttcacgttcatcagattaaagttgtaatcccatctt

SEQ ID NO: 25 - DNA, 2704 bp, Rattus sp.
gaattccggaatccggcgaggaaatacatgcactcgctgagaatcgccggcgccaggacgcagcgccacaaggtgtagcgagtgagtggggtggggcaagaggggacccaggagtccccccaggctcccagcgcgcctgctcctgctcttcaatcctgccctcggggcggacggagtgacccccgccccgaccatggtagtgttcaatggccttcttaagatcaaaatctgcgaggccgtgagcttgaagcccacagcctggtcgctgcgccatgcggtgggaccccggccccagacgttccttctggacccctacattgcccttaacgtggacgactcgcgcatcggccaaacagccaccaagcagaagaccaacagtccggcctggcacgatgagttcgtcactgatgtgtgcaatgggcgcaagatcgagctggctgtctttcacgatgctcctatcggctacgacgacttcgtggccaactgcaccatccagttcgaggagctgctgcagaatgggagccgtcacttcgaggactggattgatctggagccagaaggaaaagtctacgtgatcatcgatctctcgggatcatcgggcgaagcccctaaagacaatgaagaacgagtgtttagggagcggatgcggccaaggaagcgccaaggggctgtcaggcgcagggtccaccaggtcaatggccacaagttcatggccacctacttgcggcagcccacctactgctcccactgtagggatttcatctggggtgtcataggaaaacagggatatcaatgtcaagtttgtacctgcgtcgtccacaaacgatgccatgagctcattattacgaagtgcgctgggctaaagaaacaggaaacccctgacgaggtgggctcccaacgcttcagcgtcaacatgccccacaagttcgggatccacaactacaaggtccccacgttctgtgaccactgtggctccctgctctggggcctcttgcggcagggcctgcagtgtaaagtctgcaaaatgaatgttcaccgtcgatgcgagaccaacgtggctcccaattgtggggtggacgccagaggaattgccaaggtgctggccgatcttggcgttactccagacaaaatcaccaacagtggccagagaaggaaaaagctcgctgctggtgctgagtccccacagccggcttctggaaactccccatcagaagacgaccgatccaagtcagcgcccacctccccttgtgaccaggaactaaaagaacttgaaaacaacatccggaaggccttgtcatttgacaaccgaggagaggagcaccgagcctcgtcgtctactgatggccagctggcaagccctggcgagaacggtgaagtccggcaaggccaggccaagcgcttgggcctggatgagttcaacttcatcaaggtgttaggcaaaggcagctttggcaaggtcatgctggccgagctcaagggtaaggatgaagtctatgctgtgaaggtcttaaagaaggacgtcatcctgcaggatgacgacgtggactgcacgatgacagagaagaggattttggctctggcgcggaaacacccttatctaacccaactctattgctgcttccagaccaaggaccggctcttcttcgtcatggaatatgtaaacggtggagacctcatgttccagattcagcggtcccgaaaattcgatgagcctcgttccgggttctatgctgccgaggtcacatctgctctcatgtttctccaccaacatggagtgatctacagggatttgaaactggacaacatccttctagatgcagaaggtcactccaagctggctgactttgggatgtgcaaggaagggattctgaatggcgtgacaactaccaccttctgtgggactcctgactacatagctccagagatcctgcaggagttggagtacggcccctcagtggactggtgggccctgggcgtgctgatgtacgagatgatggccgggcagcccccctttgaagctgacaacgaggacgacttgtttgaatccatccttcacgatgacgttctctaccctgtctggcttagcaaggaggctgtcagcatcctgaaagctttcatgaccaagaacccgcacaagcgcctgggctgcgtggcagcacagaacggggaagatgccatcaagcaacatccattcttcaaggagattgactgggtactgctggagcagaagaaaatgaagccccccttcaagccgagaattaaaaccaagagagatgtcaataactttgaccaagactttacccgggaagagccaatacttacacttgtggatgaagcaatcgtgaagcagatcaaccaggaagaattcaaaggcttctcctactttggtgaagacctgatgccctgagaaactgcttcacatggagttagctcactgcaaggagggtgttgagacaatcccgtgttgcagaggctcagaatgtctcgaactattcgtcctccccagagccccagtcccacatctgctctcttatttattgcatcccctcatcccaggccctgtccttccccaccctcccagtgaccagaaggccctctttggtccagactcaccaagatcacagatttgaactgcgtctgctctgtgtgcagtgctaggtctggagtagccgtccacccacaaccctgaagcagcccggaattc

SEQ ID NO: 26 - DNA, 549 bp, Rattus sp.
agaaaagaatattttattgaaacagtttctcaattaacaatggaacaaagtacaatttgattcaaacctgtccaaccagcctgaactgctaatgaaagaactcaaacacacaggggggaactgtgtaggacctttaagtctctctgccaatgtggcaaaaaaaaaaaaaaaaaaggtggagaggggtgggggtggggtagaaaagacaaaacaactgacatcaggtttgctttgcccctgcactggggtggccctacctcctgctacaggtgcaatactggaggacaggcactctaggcatggttatagtgggcaaggggctctccttcctggcaaaggacactgtcaagttgaaccctgacccctctttctttacccacaaagcttgcctgagagtgaggtggcatttttacattcacaccatgatctctgtcccacagggtcttgggaaaggggctcacagtagatagcacatttttggtcagcccagggggaagaaggaaagagcctgaagggtagattttacaggaactggggatgaccaggatg

SEQ ID NO: 27 - DNA, 1647 bp, Rattus norvegicus
cttccgaaaagtagtccacattgagcagggtggcctggtcaagcctgagagggatgacaccgagttccagcatccatgtttcctgcgtggccaggaacagctccttgagaatatcaagaggaaagtgaccagcgtgtccaccctgaagagtgaggacataaaaatacgccaggacagtgtcaccaggctgttgacagatgtgcagctgatgaaggggaaacaggagtgtatggactccaagctcctggccatgaagcacgagaacgaggccctgtggcgggaggtggccagccttcggcagaagcatgcccagcagcaaaaagttgtcaacaagctcatccaattcctgatctcactggtgcagtcgaaccggatcctgggggtgaaaagaaagatccctctgatgttgagtgacagcagctcagcacactctgtgcccaagtatggtcgacagtactccctggagcatgtccatggtcccggcccatactcagctccatctccagcctacagcagctctagcctttactcctctgatgctgtcaccagctctgggcccataatctccgatatcactgagctggctcccaccagccctttggcctccccaggcaggagcatagatgagaggcctctgtccagcagcaccctggtccgtgtcaaggaagagccccccagcccacctcatagccctcgggtactggaggcaagccctgggcgcccagcctccatggatacccctttgtccccaactgccttcattgactccatccttcgagagagcgaacctacccctgctgcctcaaacacagcccctatggacacaaccggagcccaagcccctgcacacccagccccctccacccctgagaagtgcctcagcgtagcctgcctagacaagaacgagctaagtgatcacctggatgccatggactccaacctggacaacctgcagaccatgctgacaagccacggcttcagtgtggacaccagtgccctgctggacctgttcagcccctcggtgaccatgcccgacatgagcctgcctgacctggacagcagcctggccagcattcaggaacttctatctccccaagagcctcccaggcctattgaggcagagaatagtaacgccgactcaggaaagcagctggtgcactacacggctcagcctctgttcctgctggaccctgatgctgtggacacagggagcagtgaactgcctgtgctctttgagctgggggagagctcctacttctctgagggagatgactacacggatgatcccaccatctctcttctgacgggcactgaaccccacaaagccaaggaccccactgtctcctagaggtttcagttgtcaggctggcttgggcctggcccccagcctattcctaggacacggctggtcctggggagacaagacagttgggtagtccagggaatcctaggtcaagccagcacaaccccagtggagcacagatgggacttgggcttgggcagtaccttggatcaagaggaagatcctgaaggctgcatacctgctgccttcaccccagccccaagtctgctctctggtcagagcttcacagccacacttggactgaccctgcaggttgttcataaaattgtattttgatttttaat

SEQ ID NO: 28 - DNA, 21 bp, Artificial sequence
  Description of artificial sequence Primer
atatgatcgcctgcttattca

SEQ ID NO: 29 - DNA, 21 bp, Artificial sequence
  Description of artificial sequence Primer
aaaggtaggcaacattttcac

SEQ ID NO: 30 - DNA, 20 bp, Artificial sequence
  Description of artificial sequence Primer
ttgaccacatcgccgaatgc

SEQ ID NO: 31 - DNA, 22 bp, Artificial sequence
  Description of artificial sequence Primer
agtgcccacaatgagaccaatc

SEQ ID NO: 32 - DNA, 17 bp, Artificial Sequence
  Description of artificial sequence Primer
cccgtccttgtctccag

SEQ ID NO: 33 - DNA, 21 bp, Artificial sequence
  Description of artificial sequence Primer
agaaaaagagtgcggatgatg

SEQ ID NO: 34 - DNA, 24 bp, Artificial sequence
  Description of artificial sequence Primer
agcattttccagaagagtggtgtc

SEQ ID NO: 35 - DNA, 20 bp, Artificial sequence
  Description of artificial sequence Primer
acaaagacgctgatggctgc

SEQ ID NO: 36 - DNA, 20 bp, Artificial sequence
  Description of artificial sequence Primer
gagcaccaaaccacttcctc

SEQ ID NO: 37 - DNA, 20 bp, Artificial sequence
  Description of artificial sequence Primer
ctacaacataagggggtctc

SEQ ID NO: 38 - DNA, 23 bp, Artificial sequence
  Description of artificial sequence Primer
aaatccacttcaacctaccgctc

SEQ ID NO: 39 - DNA, 24 bp, Artificial sequence
  Description of artificial sequence Primer
tcgtctacaacataagggggtctc

SEQ ID NO: 40 - DNA, 21 bp, Artificial sequence
  Description of artificial sequence Primer
tctatgacagcgagtatgata

SEQ ID NO: 41 - DNA, 21 bp, Artificial sequence
  Description of artificial sequence Primer
caagggcactgtggaccagat

SEQ ID NO: 42 - DNA, 22 bp, Artificial sequence
  Description of artificial sequence Primer
taaccaccgcatctcttccctg

SEQ ID NO: 43 - DNA, 22 bp, Artificial sequence
  Description of artificial sequence Primer
tgtgccaaggatttcaaactgg

SEQ ID NO: 44 - DNA, 21 bp, Artificial sequence
  Description of artificial sequence Primer
cagaatgggagccgtcacttc

SEQ ID NO: 45 - DNA, 21 bp, Artificial sequence
  Description of artificial sequence Primer
agcgcacttcgtaataatgag

SEQ ID NO: 46 - DNA, 22 bp, Artificial sequence
  Description of artificial sequence Primer
tttgacaaccgaggagaggagc

SEQ ID NO: 47 - DNA, 23 bp, Artificial sequence
  Description of artificial sequence Primer
ccttggtctggaagcagcaatag

SEQ ID NO: 48 - DNA, 20 bp, Artificial sequence
  Description of artificial sequence Primer
ggggaactgtgtaggacctt

SEQ ID NO: 49 - DNA, 21 bp, Artificial sequence
  Description of artificial sequence Primer
atgtaaaaatgccacctcact

SEQ ID NO: 50 - DNA, 21 bp, Artificial sequence
  Description of artificial sequence Primer
ttcaaacctgtccaaccagcc

SEQ ID NO: 51 - DNA, 23 bp, Artificial sequence
  Description of artificial sequence Primer
ttgtgggtaaagaaagaggggtc

SEQ ID NO: 52 - DNA, 22 bp, Artificial sequence
  Description of artificial sequence Primer
tcgcatccgtgtctactccatc

SEQ ID NO: 53 - DNA, 23 bp, Artificial sequence
  Description of artificial sequence Primer
ggaagtccttttctgtcaccacc

SEQ ID NO: 54 - DNA, 21 bp, Artificial sequence
  Description of artificial sequence Primer
cagaaccaccccatttacctg

SEQ ID NO: 55 - DNA, 24 bp, Artificial sequence
  Description of artificial sequence Primer
tgtttacatccaaggctctcactg

SEQ ID NO: 56 - DNA, 24 bp, Artificial sequence
  Description of artificial sequence Primer
cgaaagaggatgtggatgaagatg

SEQ ID NO: 57 - DNA, 24 bp, Artificial sequence
  Description of artificial sequence Primer
atgtatcggagtcgcttggtgagg

SEQ ID NO: 58 - DNA, 22 bp, Artificial sequence
  Description of artificial sequence Primer
aggatgtggatgaagatgtgcc

SEQ ID NO: 59 - DNA, 24 bp, Artificial sequence
  Description of artificial sequence Primer
atgtatcggagtcgcttggtgagg

SEQ ID NO: 60 - DNA, 23 bp, Artificial sequence
  Description of artificial sequence Primer
ggaggatgcccgagttttacaac

SEQ ID NO: 61 - DNA, 24 bp, Artificial sequence
  Description of artificial sequence Primer
aagaggtccaaggatgtgctgtgg

SEQ ID NO: 62 - DNA, 22 bp, Artificial sequence
  Description of artificial sequence Primer
aagatggcactacccacagcac

SEQ ID NO: 63 - DNA, 20 bp, Artificial sequence
  Description of artificial sequence Primer
tttccttccacccgttcctg

SEQ ID NO: 64 - DNA, 22 bp, Artificial sequence
  Description of artificial sequence Primer
atcaaggatgctggaggaaggg

SEQ ID NO: 65 - DNA, 22 bp, Artificial sequence
  Description of artificial sequence Primer
tagttcagggcactgttcgtgg

SEQ ID NO: 66 - DNA, 20 bp, Artificial sequence
  Description of artificial sequence Primer
cttgggtatcactgctttga

SEQ ID NO: 67 - DNA, 21 bp, Artificial sequence
  Description of artificial sequence Primer
ataatactccgcctctgcttc

SEQ ID NO: 68 - DNA, 27 bp, Artificial sequence
  Description of artificial sequence Primer
tgttctggtggcgactgccagacacct

SEQ ID NO: 69 - DNA, 27 bp, Artificial sequence
  Description of artificial sequence Primer
tatcttttcaatcccttcaagaagccg

SEQ ID NO: 70 - DNA, 22 bp, Artificial sequence
  Description of artificial sequence Primer
aagaactgctgggggaagattg

SEQ ID NO: 71 - DNA, 20 bp, Artificial sequence
  Description of artificial sequence Primer
ttgccgtgagccttgacctg

SEQ ID NO: 72 - DNA, 24 bp, Artificial sequence
  Description of artificial sequence Primer
acaactacagcacaggctacgacg

SEQ ID NO: 73 - DNA, 22 bp, Artificial sequence
  Description of artificial sequence Primer
agaagagtgaaaggcgggagac

SEQ ID NO: 74 - DNA, 21 bp, Artificial sequence
  Description of artificial sequence Primer
gacgatccgggctcccttcac

SEQ ID NO: 75 - DNA, 21 bp, Artificial sequence
  Description of artificial sequence Primer
atggatgccggcttgcgaatg

SEQ ID NO: 76 - DNA, 23 bp, Artificial sequence
  Description of artificial sequence Primer
gaccactttggcagacttcactg

SEQ ID NO: 77 - DNA, 23 bp, Artificial sequence
  Description of artificial sequence Primer
ccttccatccttcacagataggg

SEQ ID NO: 78 - DNA, 21 bp, Artificial sequence
  Description of artificial sequence Primer
gcctgatatcgaccgaacagc

SEQ ID NO: 79 - DNA, 21 bp, Artificial sequence
  Description of artificial sequence Primer
atcatcttccttttggcaagc

SEQ ID NO: 80 - DNA, 22 bp, Artificial sequence
  Description of artificial sequence Primer
acttccatcctatccagcctgc

SEQ ID NO: 81 - DNA, 23 bp, Artificial sequence
  Description of artificial sequence Primer
caccacaaacacaatgccatctg

SEQ ID NO: 82 - DNA, 19 bp, Artificial sequence
  Description of artificial sequence Primer
gcaattccaaaacagtcac

SEQ ID NO: 83 - DNA, 21 bp, Artificial sequence
  Description of artificial sequence Primer
tttagcccatctcctatgatt

SEQ ID NO: 84 - DNA, 24 bp, Artificial sequence
  Description of artificial sequence Primer
aggctacgctgtctaccagattcc

SEQ ID NO: 85 - DNA, 23 bp, Artificial sequence
  Description of artificial sequence Primer
aacaccctcaagcagaagtcacc

SEQ ID NO: 86 - DNA, 18 bp, Artificial sequence
  Description of artificial sequence Primer
tggacacggggttctaca

SEQ ID NO: 87 - DNA, 21 bp, Artificial sequence
  Description of artificial sequence Primer
caactccttgggaatctggta

SEQ ID NO: 88 - DNA, 22 bp, Artificial sequence
  Description of artificial sequence Primer
ggagccattgttcacattaccg

SEQ ID NO: 89 - DNA, 24 bp, Artificial sequence
  Description of artificial sequence Primer
taccctgccttcttctctctggag

SEQ ID NO: 90 - DNA, 19 bp, Artificial sequence
  Description of artificial sequence Primer
ccgaccagcaacacagagc

SEQ ID NO: 91 - DNA, 21 bp, Artificial sequence
  Description of artificial sequence Primer
ttcgccgtaaaacatcagcat

SEQ ID NO: 92 - DNA, 24 bp, Artificial sequence
  Description of artificial sequence Primer
ggagcagttttgtgtgtgtgattc

SEQ ID NO: 93 - DNA, 25 bp, Artificial sequence
  Description of artificial sequence Primer
tgcggtagagtaagcatacaggaag

SEQ ID NO: 94 - DNA, 21 bp, Artificial sequence
  Description of artificial sequence Primer
gccgaaactcttcatcattca

SEQ ID NO: 95 - DNA, 21 bp, Artificial sequence
  Description of artificial sequence Primer
gatctgtttctttgcgtggaa

SEQ ID NO: 96 - DNA, 23 bp, Artificial sequence
  Description of artificial sequence Primer
tggaactgaagggtgacattgag

SEQ ID NO: 97 - DNA, 22 bp, Artificial sequence
  Description of artificial sequence Primer
atggcttggcagagagggattc

SEQ ID NO: 98 - DNA, 21 bp, Artificial sequence
  Description of artificial sequence Primer
gaactgaagggtgacattgag

SEQ ID NO: 99 - DNA, 21 bp, Artificial sequence
  Description of artificial sequence Primer
gggatgtttagtttcctccac

SEQ ID NO: 100 - DNA, 23 bp, Artificial sequence
  Description of artificial sequence Primer
ctgacacaggacacggaacaaag

SEQ ID NO: 101 - DNA, 25 bp, Artificial sequence
  Description of artificial sequence Primer
ggtgacactcttacattgagatgcc

SEQ ID NO: 102 - DNA, 19 bp, Artificial sequence
  Description of artificial sequence Primer
cacagttctcgggtggagt

SEQ ID NO: 103 - DNA, 21 bp, Artificial sequence
  Description of artificial sequence Primer
cattgagatgccctaacagtg

SEQ ID NO: 104 - DNA, 23 bp, Artificial sequence
  Description of artificial sequence Primer
gaccactggaaggaagaaacacc

SEQ ID NO: 105 - DNA, 25 bp, Artificial sequence
  Description of artificial sequence Primer
cagactcaccgaatgacacactctc

SEQ ID NO: 106 - DNA, 25 bp, Artificial sequence
  Description of artificial sequence Primer
gagagtgtgtcattcggtgagtctg

SEQ ID NO: 107 - DNA, 25 bp, Artificial sequence
  Description of artificial sequence Primer
cggaagagtccataggtgtgaagtc

SEQ ID NO: 108 - DNA, 21 bp, Artificial sequence
  Description of artificial sequence Primer
tggctcggaaatacgcagttg

SEQ ID NO: 109 - DNA, 24 bp, Artificial sequence
  Description of artificial sequence Primer
aggtgtgtgtcctccatacagtgc

SEQ ID NO: 110 - DNA, 22 bp, Artificial sequence
  Description of artificial sequence Primer
ggtggcttcaacttctatgcgg

SEQ ID NO: 111 - DNA, 22 bp, Artificial sequence
  Description of artificial sequence Primer
ccagggattggtgaggttttcc

(19) United States
(12) Patent Application Publication
(10) Pub. No.: US 2005/0064462 A1
(43) Pub. Date: Mar. 24, 2005

(54) Methods, compositions, and kits for predicting the effect of compounds on hot flash symptoms

(76) Inventors: Bernd Stein, San Diego, CA (US); Helen Brady, San Diego, CA (US); Dan Zhu, San Diego, CA (US)

Correspondence Address: JONES DAY, 222 EAST 41ST ST, NEW YORK, NY, 10017, US

(21) Appl. No.: 10/870,387

(22) Filed: Jun. 16, 2004

Related U.S. Application Data
(60) Provisional application No. 60/479,570, filed on Jun. 17, 2003.

(51) Int. Cl. C12Q 1/68; G01N 33/53; G01N 33/567; A61K 31/56; A61K 31/445; A61K 31/138

(52) U.S. Cl. 435/6; 435/7.2; 514/182; 514/319; 514/651

(57) ABSTRACT

The present invention provides methods, compositions, and kits, for determining the effects of one or more candidate compounds on hot flash symptoms. In certain aspects, the methods, compositions, and kits can be used to identify compounds that decrease the incidence of hot flash symptoms. In other aspects, the methods, compositions, and kits can be used to determine whether candidate compounds increase the incidence of undesirable hot flash symptoms when administered to a subject.

### 1. REFERENCE TO RELATED APPLICATIONS

The present application is entitled to and claims the benefit of U.S. Provisional Application No. 60/479,570, filed Jun. 17, 2003, which application is hereby incorporated by reference in its entirety.

### 2. FIELD OF THE INVENTION

The field of the invention generally relates to the identification of the effect of candidate compounds on hot flash symptoms based upon their effect on the regulation of expression of genes associated with hot flash symptoms. More specifically, the invention relates to identification of compounds that decrease hot flash symptoms and/or determining the effects of candidate compounds on hot flash symptoms. In another aspect, the invention provides methods of determining whether candidate compounds increase the incidence of hot flash symptoms as an undesirable side effect.

### 3. BACKGROUND

Hot flash symptoms are physical sensations that are experienced by subjects, most commonly by women undergoing menopause. As many as 67% to 75% of women undergoing physiological or surgical menopause report experiencing hot flash symptoms. See Agarwal and Judd, 1995, Osteoporosis: Etiology, Diagnosis, and Management, Riggs et al., eds., Ch. 16. “Management of Menopause,” pp. 351-354. Such symptoms generally include a sudden sensation of heat radiating from the face, neck, and/or chest; chills; sweating or perspiration; anxiety; tingling; and/or pressure in the head. See id. Hot flash symptoms generally do not present serious medical issues; however, approximately 10% to 15% of menopausal women experience hot flash symptoms of sufficient severity to seek medical treatment. See id.

Hot flash symptoms can also be caused as a side effect by compounds that modulate the estrogen receptor. For example, raloxifene, commonly administered in the treatment of osteoporosis, causes approximately 11% of subjects to experience hot flash symptoms of sufficient severity to discontinue therapy. See Drug Facts and Comparisons, 2003, Wickersham et al., eds., Wolters Lewis Health, St. Louis, Mo., pp. 225-227.

Hot flash symptoms are believed to be mediated by endocrinological changes occurring in menopausal women that likely involve downward resetting of the central thermoregulatory mechanism. See Kronenberg and Downey, 1987, Can. J. Physol. Pharmacol. 65:1312-1324. These changes are believed to be mediated by the hypothalamus and/or the pituitary gland. See id.

Hot flash symptoms have generally been treated with estrogen replacement therapy. See Agerwal and Judd, supra. Unfortunately, estrogen replacement therapy presents several serious side effects, including an increased risk of endometrial, ovarian, and/or breast cancer; cardiovascular disorders; hypercalcemia; glucose tolerance; depression, and hypothyroidism, among others. See Drug Facts and Comparisons, 2003, Wickersham et al., eds., Wolters Lewis Health, St. Louis, Mo., pp. 217-219.

Thus, there remains an unmet need for compounds that can be used to treat hot flash symptoms without the deleterious side effects presented by standard estrogen replacement therapy. In addition, rapid, inexpensive methods for determining the effects on hot flash symptoms of compounds indicated for the treatment of, for example, osteoporosis to identify therapeutic agents that do not increase the incidence of hot flash symptoms are also needed.

### 4. SUMMARY OF THE INVENTION

The present invention provides methods, compositions, and kits for determining the effects of candidate compounds on hot flash symptoms. Further, the invention provides methods, compositions, and kits for identifying candidate compounds that decrease hot flash symptoms. The methods and kits of the invention are based, in part, on the recognition of a correlation between the regulation of expression of certain genes by a cell and contact by the cell with compounds known to increase or decrease the incidence of hot flash symptoms. The methods and kits are described in reference to the embodiments of the invention that follow.

In certain aspects, the invention provides a method for determining the effect of a candidate compound on hot-flash symptoms that comprises contacting a first cell that expresses an estrogen receptor or estrogen related receptor with the candidate compound and determining the effect of the candidate compound on the first cell's expression of a panel of genes associated with hot flash symptoms.

In certain embodiments, the methods further comprise comparing the first cell's expression of the panel of genes associated with hot flash symptoms with a reference expression profile of the panel of genes associated with hot flash symptoms. In certain embodiments, the expression profile of the panel of genes can be the expression profile of the panel of genes following contacting the cell with a compound selected from the group consisting of estradiol, tibolone, raloxifene, and 4-hydroxy tamoxifen.

In other embodiments, the methods further comprise comparing the first cell's expression of the panel of genes associated with hot flash symptoms with a second cell's expression of the panel of genes associated with hot flash symptoms following contact with a compound that has a known effect on hot flash symptoms. The compound that has a known effect on hot flash symptoms can be any such compound known to one of skill in the art without limitations. In certain embodiments, the compound that has a known effect on hot flash symptoms can be selected from the group that consists of estradiol, tibolone, raloxifene, and 4-hydroxy tamoxifen.

In certain embodiments, the methods of the invention further comprise determining that the candidate compound decreases the incidence of hot flash symptoms.

In certain embodiments, the cell that expresses an estrogen receptor can express estrogen receptor α. In other embodiments, the cell that expresses an estrogen receptor can express estrogen receptor β. In certain embodiments, the cell that expresses the estrogen receptor can express both estrogen receptor a and estrogen receptor β. In certain embodiments, the cell that expresses the estrogen related receptor can express estrogen related receptor α. In other embodiments, the cell that expresses the estrogen related receptor can express estrogen related receptor β. In yet other embodiments, the cell that expresses the estrogen related receptor can express estrogen related receptor γ. In still other embodiments, the cell that expresses the estrogen related receptor can express two or three estrogen related receptors, each of which is selected from the group that consists of estrogen related receptor α, estrogen related receptor β, and estrogen related receptor γ.

Any cell known by one of skill in the art to express the estrogen receptor or the estrogen related receptor, without limitation, can be used in the methods and kits of the invention. In certain embodiments, the cell that expresses the estrogen receptor or estrogen related receptor can be selected from the group consisting of a pituitary cell and a hypothalamus cell. In further embodiments, the cell that expresses the estrogen receptor can be a GH3 cell, a GH4 cell, a PR1 cell, a MtT/E-2 cell, a alphaT3-1 cell, a D12 cell, an RCF-8 cell, and a GT1-7 cell. In certain embodiments, the cell that expresses the estrogen related receptor can be selected from the group that consists of an A172 glioma cell, a MCF10a cell, a MCF12 cell, a MDA-MB-231 cell, a MDA-MB-435 cell, a MDA-MB-436 cell, a MDA-MB-468 cell, a Hs 578T cell, a BT 20 cell, a BT 474 cell, a BT 549 cell, a SKBr 3 cell, a ZR 75.1 cell, a T47D cell, and a MCF7 cell.

Any technique known by one of skill in the art to be useful in quantifying expression of a panel of genes associated with hot flash symptoms can be used in the methods of the invention, without limitation. In certain embodiments, the cell's expression of the panel of genes associated with hot flash symptoms can be quantified by a technique selected from the group of reverse transcription real time PCR, quantitative reverse transcription PCR, Northern blot assays, dot blot assays, reverse dot blot assays, RNAse protection assays, 5′-nuclease assays, reporter gene assays, branched DNA assays, bead array assays, and multiplexed array mRNA assays. In a preferred embodiment, the technique used to quantify the expression of the panel of genes associated with hot flash symptoms is a multiplexed array mRNA assay.

The panel of genes that is associated with hot flash symptoms can include any gene known by one of skill in the art to be associated with hot flash symptoms, without limitation. In certain embodiments, at least one member of the panel of genes that is associated with hot flash symptoms can be selected from the group consisting of Activin Beta E, Type II Hexokinase, Multi Drug Resistance Gene, Parvalbumin, BAD2, Prolactin, Argininosuccinate Synthetase, Ribonucleoside Reductase 1, Interleukin-18, ARL gene 4, Calpain, EST196325, CPP32, EST208064, 2-alpha-1 globin, Amiloride Binding Protein, Annexin 1, N27, HBP1, D-binding protein, FE65, Protein Kinase C type I, Glutamate Receptor subunit d1, VAP1, Protein Kinase C subspecies epsilon, EST203549, and Heat Shock Transcription Factor 1.

In a more preferred embodiment, at least one member of the panel of genes that is associated from hot flash symptoms can be selected from the group consisting of Type II Hexokinase, Multi Drug Resistance Gene, Parvalbumin, BAD2, Interleukin-18, Calpain, EST196325, Annexin 1, N27, HBP1, and Protein Kinase C subspecies epsilon.

In certain embodiments, expression of at least one member of the panel of genes can be upregulated in a cell that expresses the estrogen receptor or estrogen related receptor following contact with the candidate compound. In certain embodiments, expression of at least one member of the panel of genes can be not upregulated in a cell that expresses the estrogen receptor or estrogen related receptor following contact with the candidate compound. In certain embodiments, expression of at least one member of the panel of genes can be downregulated in a cell that expresses the estrogen receptor or estrogen related receptor following contact with the candidate compound. In certain embodiments, expression of at least one member of the panel of genes can be not downregulated in a cell that expresses the estrogen receptor or estrogen related receptor following contact with the candidate compound.

In certain embodiments, expression of Type II Hexokinase, Multi Drug Resistance Gene, Parvalbumin, BAD2, Prolactin, Interleukin-18, Calpain, and EST196325 can be upregulated in a cell that expresses the estrogen receptor or estrogen related receptor following contact with the candidate compound. In certain embodiments, expression of Annexin 1, N27, and HBP1 can be not upregulated in a cell that expresses the estrogen receptor or estrogen related receptor following contact with the candidate compound. In certain embodiments, expression of Protein Kinase C subspecies epsilon can be not downregulated in a cell that expresses the estrogen receptor or estrogen related receptor following contact with the candidate compound.

In a preferred embodiment, expression of Type II Hexokinase, Multi Drug Resistance Gene, Parvalbumin, BAD2, Prolactin, Interleukin-18, Calpain, and EST196325 are upregulated in a cell that expresses the estrogen receptor or estrogen related receptor following contact with the candidate compound; expression of Annexin 1, N27, and HBP1 is not upregulated in a cell that expresses the estrogen receptor or estrogen related receptor following contact with the candidate compound; and expression of Protein Kinase C subspecies epsilon is not downregulated in a cell that expresses the estrogen receptor or estrogen related receptor following contact with the candidate compound.

The candidate compound can be any compound known by one of skill in the art without limitation. In certain embodiments, the candidate compound can be an estrogen receptor modulator. In further embodiments, the estrogen receptor modulator can be a selective estrogen receptor modulator.

The estrogen receptor modulator can be effective to treat any disease, condition, affliction, or disorder known by one of skill in the art to be treatable with an estrogen receptor modulator, without limitation. In certain embodiments, the estrogen receptor modulator can be therapeutically effective to treat or prevent osteoporosis. In other embodiments, the selective estrogen receptor modulator can be therapeutically effective to treat or prevent post-menopausal symptoms. In still other embodiments, the selective estrogen receptor modulator can be therapeutically effective to treat or prevent a proliferative disorder.

The proliferative disorder can be any proliferative disorder known by one of skill in the art to be treatable with an estrogen receptor modulator without limitation. In certain embodiments, the proliferative disorder can be endometriosis. In other embodiments, the proliferative disorder can be cancer. In certain embodiments, the cancer can be selected from the group consisting of breast cancer, uterine cancer, ovarian cancer, cervical cancer, testicular cancer, and prostate cancer.

In another aspect, the invention provides a method for rapidly determining the effects of a plurality of compounds on hot-flash symptoms, comprising separately contacting a sample of cells that express an estrogen receptor or estrogen related receptor with each member of the plurality of compounds; and assessing the effect of each member of the plurality of compounds on each of the samples of cells' expression of a panel of genes associated with hot flash symptoms, thereby predicting the effect of each of the compounds on hot-flash symptoms.

In certain embodiments, the method further comprises comparing the expression of the panel of genes associated with hot flash symptoms by the samples of cells with a reference expression profile of the panel of genes associated with hot flash symptoms. In certain embodiments, the reference expression profile of the panel of genes can be the expression profile of the panel of genes following contacting the cell with a compound selected from the group consisting of estradiol, tibolone, raloxifene, and tamoxifen.

In other embodiments, the method further comprises comparing the expression of the panel of genes associated with hot flash symptoms by the samples of cells with the expression of the panel of genes associated with hot flash symptoms by a sample of cells following contact with a compound that has a known effect on hot flash symptoms. In certain embodiments, the compound that has a known effect on hot flash symptoms can be selected from the group that consists of estradiol, tibolone, raloxifene, and tamoxifen.

In certain embodiments, the methods further comprise determining that the compound decreases the incidence of hot flash symptoms.

In certain embodiments, the sample of cells that expresses the estrogen receptor can estrogen receptor α. In other embodiments, the sample of cells that expresses the estrogen receptor can express estrogen receptor β. In yet other embodiments, the sample of cells that expresses the estrogen receptor can express both estrogen receptor α and estrogen receptor β. In certain embodiments, the sample of cells that expresses the estrogen related receptor can express estrogen related receptor α. In other embodiments, the sample of cells that expresses the estrogen related receptor can express estrogen related receptor β. In yet other embodiments, the sample of cells that expresses the estrogen related receptor can express estrogen related receptor γ. In still other embodiments, the sample of cells that expresses the estrogen related receptor can express two or three estrogen related receptors, each of which is selected from the group that consists of estrogen related receptor α, estrogen related receptor β, and estrogen related receptor γ.

In certain embodiments, the cell that expresses the estrogen receptor can be selected from the group consisting of a pituitary cell and a hypothalamus cell. In certain embodiments, the cell that expresses the estrogen receptor can be selected from the group consisting of a GH3 cell, a GH4 cell, a PR1 cell, a MtT/E-2 cell, a alphaT3-1 cell, a D12 cell, an RCF-8 cell, and a GT1-7 cell.

In certain embodiments, the expression of the panel of genes associated with hot flash symptoms by the sample of cells can be quantified by determining the presence and amount of mRNA expressed from the panel of genes. In certain embodiments, the expression of the panel of genes associated with hot flash symptoms by the sample of cells can be quantified by a technique selected from the group of reverse transcription real time PCR, quantitative reverse transcription PCR, Northern blot assays, dot blot assays, reverse dot blot assays, RNAse protection assays, 5′-nuclease assays, reporter gene assays, branched DNA assays, bead array assays, and multiplexed array mRNA assays. In certain embodiments, the expression of the panel of genes associated with hot flash symptoms can be quantified by a multiplexed array mRNA assay.

In certain embodiments, the expression of the panel of genes associated with hot flash symptoms by the sample of cells can be quantified by determining the presence and amount of protein expressed from the panel of genes. In certain embodiments, the expression of the panel of genes associated with hot flash symptoms can be quantified by a technique selected from the group of a western blot assay, an ELISA assay, a cytokine bead array, multiplexed protein detection assays, and an immunofluorescence assay.

In certain embodiments, at least one member of the panel of genes can be selected from the group consisting of Activin Beta E, Type II Hexokinase, Multi Drug Resistance Gene, Parvalbumin, BAD2, Prolactin, Argininosuccinate Synthetase, Ribonucleoside Reductase 1, Interleukin-18, ARL gene 4, Calpain, EST196325, CPP32, EST208064, 2-alpha-1 globin, Amiloride Binding Protein, Annexin 1, N27, HBP1, D-binding protein, FE65, Protein Kinase C type I, Glutamate Receptor subunit d1, VAPI, Protein Kinase C subspecies epsilon, EST203549, and Heat Shock Transcription Factor 1. In certain embodiments, at least one member of the panel of genes can be selected from the group consisting of Type II Hexokinase, Multi Drug Resistance Gene, Parvalbumin, BAD2, Interleukin-18, Calpain, EST196325, Annexin 1, N27, HBP1, and Protein Kinase C subspecies epsilon, and expression of at least one member of the panel of genes can be upregulated in the cell following contact with the candidate compound.

In certain embodiments, at least one member of the panel of genes can be selected from the group consisting of Type II Hexokinase, Multi Drug Resistance Gene, Parvalbumin, BAD2, Interleukin-18, Calpain, EST196325, Annexin 1, N27, HBP1, and Protein Kinase C subspecies epsilon, and expression of at least one member of the panel of genes can be not upregulated in the cell following contact with the candidate compound.

In certain embodiments, at least one member of the panel of genes cam be selected from the group consisting of Type II Hexokinase, Multi Drug Resistance Gene, Parvalbumin, BAD2, Prolactin, Interleukin-18, Calpain, EST196325, Annexin 1, N27, HBP1, and Protein Kinase C subspecies epsilon, and expression of at least one member of the panel of genes can be downregulated in the cell following contact with the candidate compound.

In certain embodiments, at least one member of the panel of genes can be selected from the group consisting of Type II Hexokinase, Multi Drug Resistance Gene, Parvalbumin, BAD2, Prolactin, Interleukin-18, Calpain, EST196325, Annexin 1, N27, HBP1, and Protein Kinase C subspecies epsilon, and expression of at least one member of the panel of genes can be not downregulated in the cell following contact with the candidate compound.

In certain embodiments, the panel of genes comprises Type II Hexokinase, Multi Drug Resistance Gene, Parvalbumin, BAD2, Prolactin, Interleukin-18, Calpain, EST196325, Annexin 1, N27, and HBP1; expression of Type II Hexokinase, Multi Drug Resistance Gene, Parvalbumin, BAD2, Prolactin, Interleukin-18, Calpain, and EST196325 can be upregulated in the cell following contact with the candidate compound; expression of Annexin 1, N27, and HBP1 can be not upregulated in the cell following contact with the candidate compound; and expression of Protein Kinase C subspecies epsilon can be not downregulated in the cell following contact with the candidate compound.

In another aspect, the invention provides kits for determining the effect of a compound on hot flash symptoms. The kits generally comprise at least one primer or probe that can be used to detect the presence and amount of an expression product of a member of a panel of genes associated with hot flash symptoms. In certain embodiments, the primer or probe can be used to detect an mRNA expressed from a member of a panel of genes associated with hot flash symptoms. In other embodiments, the primer or probe can be used to detect a protein expressed from a member of a panel of genes associated with hot flash symptoms.

In a preferred embodiment, the kits can comprise at least one gene-specific nuclease protection probe that is specific for a member of a panel of genes associated with hot flash symptoms and that can be directly or indirectly detectable; and a surface having multiple spatially discrete regions, at least two of which regions are substantially identical, and wherein the regions are adapted to specifically bind to the gene-specific protection probe(s).

In yet another aspect, the invention provides compositions suitable for determining the effect of a compound on hot flash symptoms. The compositions may also be used in the methods and kits of the invention. The compositions of the invention generally comprise at least one primer or probe that can be used to detect the presence and amount of an expression product of a member of a panel of genes associated with hot flash symptoms and a suitable buffer, diluent, or excipient. The member of the panel of genes can be any member of a panel of genes associated with hot flash symptoms known by one of skill in the art without limitation.

In certain embodiments, the primer or probe can be used to detect an mRNA expressed from a member of a panel of genes associated with hot flash symptoms. In other embodiments, the primer or probe can be used to detect a protein expressed from a member of a panel of genes associated with hot flash symptoms. In a preferred embodiment, the composition comprises a plurality of gene-specific nuclease protection probes, wherein each member of the plurality of gene-specific nuclease protection probes hybridizes under stringent conditions to an mRNA expressed from a member of a panel of genes associated with hot flash symptoms.

In still another aspect, the invention provides arrays useful for the identification of the effect of a plurality of compounds on hot flash symptoms. In certain embodiments, the array can comprise a non-porous surface; and a plurality of different oligonucleotides connected with the surface, wherein at least one of the oligonucleotides hybridizes under stringent conditions to a member of a panel of genes associated with hot flash symptoms, and wherein each of the different oligonucleotides is connected with the surface in a different predetermined region of the surface. The member of the panel of genes can be any member of a panel of genes associated with hot flash symptoms known by one of skill in the art, without limitation.

### 5. BRIEF DESCRIPTION OF THE FIGURES

FIG. 1A presents the effects of estradiol, raloxifene, tibolone, and 4-hydroxy tamoxifen on expression of BAD2 and MDR in GH3 cells;

FIG. 1B presents the effects of estradiol, raloxifene, tibolone, and 4-hydroxy tamoxifen on expression of Prolactin and Parvalbumin in GH3 cells;

FIG. 1C presents the effects of estradiol, raloxifene, tibolone, and 4-hydroxy tamoxifen on expression of HKII and Calpain in GH3 cells;

FIG. 1D presents the effects of estradiol, raloxifene, tibolone, and 4-hydroxy tamoxifen on expression of Il-18 and Annexin 1 in GH3 cells; and

FIG. 1E presents the effects of estradiol, raloxifene, tibolone, and 4-hydroxy tamoxifen on expression of N27 (Vdup1) and Protein Kinase C, epsilon subspecies in GH3 cells.

### 6. DETAILED DESCRIPTION OF EMBODIMENTS OF THE INVENTION

The present invention provides methods, kits, compositions, and arrays for determining the effect of candidate compounds on hot flash symptoms. The methods, kits, compositions, and arrays are described according to the various embodiments of the invention presented in detail below.

### 6.1. Definitions

The terms “reference compound(s),” “compound(s) known to affect hot flash symptoms,” and “compound(s) with a known effect on hot flash symptoms” are used interchangeably and generally refer to compounds that are known by one of skill in the art to increase or decrease the incidence of hot flash symptoms when the compounds are administered to a subject. Exemplary reference compounds include estradiol, tibolone, raloxifene, and 4-hydroxy tamoxifen.

The terms “candidate compound” or “candidate compounds” refer to one or more compounds whose effects on hot flash symptoms are to be determined according to the methods of the invention. The candidate compound can be any compound known by one of skill in the art without limitation. The candidate compound can be derived from any source known to one of skill in the art without limitation. For example, and not by way of limitation, the candidate compound can be organic or inorganic; polar or non-polar; neutrally charged, positively charged, negatively charged, or zwitterionic; a small organinc molecule or a large macromolecule, etc. Candidate compounds suitable for the methods of the invention can be obtained from any commercial source, including Aldrich (1001 West St. Paul Ave., Milwaukee, Wis. 53233), Sigma Chemical (P.O. Box 14508, St. Louis, Mo. 63178), Fluka Chemie AG (Industriestrasse 25, CH-9471 Buchs, Switzerland (Fluka Chemical Corp. 980 South 2nd Street, Ronkonkoma, N.Y. 11779)), Eastman Chemical Company, Fine Chemicals (P.O Box 431, Kingsport, Tenn. 37662), Boehringer Mannheim GmbH (Sandhofer Strasse 116, D-68298 Mannheim), Takasago (4 Volvo Drive, Rockleigh, N.J. 07647), SST Corporation (635 Brighton Road, Clifton, N.J. 07012), Ferro (111 West Irene Road, Zachary, La. 70791), Riedel-deHaen Aktiengesellschaft (P.O. Box D-30918, Seelze, Germany), PPG Industries Inc., Fine Chemicals (One PPG Place, 34th Floor, Pittsburgh, Pa. 15272). Further, the effect of any kind of natural product on expression of genes associated with hot flash symptoms may be determined according to the methods of the invention, including microbial, fungal or plant extracts.

The term “hot flash symptom(s)” refers to one or more symptoms commonly experienced by subjects, typically women, typically at menopause. Hot flash symptoms can include, but are not limited to, a sudden sensation of heat radiating from the face, neck, and/or chest; chills; sweating or perspiration; anxiety; tingling; and/or pressure in the head. Hot flash symptoms can be experienced by subjects for short, e.g., seconds, or long, e.g., hours, durations.

The term “upregulated” refers to an increase in the expression of one or more genes associated with hot flash symptoms in a cell following contact of the cell with a compound that has an known or unknown effect on hot flash symptoms.

The term “downregulated” refers to a decrease in the expression of one or more genes associated with hot flash symptoms in a cell following contact of the cell with a compound that has an known or unknown effect on hot flash symptoms.

The term “panel of genes associated with hot flash symptoms” refers to a plurality of genes, expression of each of which is modulated by a compound that has a known effect on hot flash symptoms. A panel of genes associated with hot flash symptoms can comprise as few as two genes or as many as 30, or more. In certain embodiments, the panel of genes associated with hot flash symptoms comprises 2, 3, 4, 5, 6, 7, 8, 9, 10, 12, 14, 16, 18, 20, 22, or 24 genes associated with hot flash symptoms. In a preferred embodiment, the panel of genes associated with hot flash symptoms comprises 10 genes associated with hot flash symptoms.

The term “high throughput method for determining regulation of gene expression” refers to any method known by one of skill in the art without limitation suitable for determining the effects of a large number of candidate compounds on regulation of expression of genes. Such methods include, for example, but not by way of limitation, the multiplexed array mRNA assays, differential PCR, restriction mediated differential display, AFLP-based transcript profiling, serial expression of gene analysis, Massive Parallel Signature Sequencing, dot blot assays, reverse dot blot assays, and bead array-based assays.

The term “reference expression profile,” as used herein, refers to the profile of expression of a panel of genes associated with hot flash symptoms in a cell following contact of the cell with a compound that has a known effect on hot flash symptoms. The reference expression profile can be the profile of expression of genes associated with hot flash symptoms in a cell following contact of the cell with any compound with a known effect on hot flash symptoms known by one of skill in the art without limitation. In certain embodiments, the reference expression profile is the profile of expression of genes associated with hot flash symptoms in a cell following contact of the cell with estradiol, tibolone, raloxifene, or 4-hydroxy tamoxifen.

As used herein, the terms “nucleic acid,” “nucleotide,” “polynucleotide” and “oligonucleotide” refer to primers, probes, oligomer fragments to be detected, oligomer controls and unlabeled blocking oligomers and is generic to polydeoxyribonucleotides (containing 2-deoxy-D-ribose), to polyribonucleotides (containing D-ribose), and to any other N-glycoside of a purine or pyrimidine base, or modified purine or pyrimidine bases.

A nucleic acid, nucleotide, polynucleotide or oligonucleotide can comprise phosphodiester linkages or modified linkages including, but not limited to phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

A nucleic acid, nucleotide, polynucleotide or oligonucleotide can comprise the five biologically occurring bases (adenine, guanine, thymine, cytosine and uracil) and/or bases other than the five biologically occurring bases. These bases may serve a number of purposes, e.g., to stabilize or destabilize hybridization; to promote or inhibit probe degradation; or as attachment points for detectable moieties or quencher moieties. For example, a polynucleotide of the invention can contain one or more modified, non-standard, or derivatized base moieties, including, but not limited to, N⁶-methyl-adenine, N⁶-tert-butyl-benzyl-adenine, imidazole, substituted imidazoles, 5-fluorouracil, 5-bromouracil, 5-chlorouracil, 5-iodouracil, hypoxanthine, xanthine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acidmethylester, 3-(3-amino-3-N-2-carboxypropyl) uracil, (acp3)w, 2,6-diaminopurine, and 5-propynyl pyrimidine. Other examples of modified, non-standard, or derivatized base moieties may be found in U.S. Pat. Nos. 6,001,611, 5,955,589, 5,844,106, 5,789,562, 5,750,343, 5,728,525, and 5,679,785, each of which is incorporated herein by reference in its entirety.

Furthermore, a nucleic acid, nucleotide, polynucleotide or oligonucleotide can comprise one or more modified sugar moieties including, but not limited to, arabinose, 2-fluoroarabinose, xylulose, and hexose.

It is not intended that the present invention be limited by the source of a nucleic acid, nucleotide, polynucleotide or oligonucleotide. A nucleic acid, nucleotide, polynucleotide or oligonucleotide can be from a human or non-human mammal, or any other organism, or derived from any recombinant source, synthesized in vitro or by chemical synthesis. A nucleic acid, nucleotide, polynucleotide or oligonucleotide may be DNA, RNA, cDNA, DNA-RNA, locked nucleic acid (LNA), peptide nucleic acid (PNA), a hybrid or any mixture of the same, and may exist in a double-stranded, single-stranded or partially double-stranded form. A nucleic acid may also be a derivative nucleic acid as described in U.S. Pat. No. 5,696,248, which is hereby incorporated by reference in its entirety. The nucleic acids of the invention include both nucleic acids and fragments thereof, in purified or unpurified forms, including genes, chromosomes, plasmids, the genomes of biological material such as microorganisms, e.g., bacteria, yeasts, viruses, viroids, molds, fungi, plants, animals, humans, and the like.

There is no intended distinction in length between the terms nucleic acid, nucleotide, polynucleotide and oligonucleotide, and these terms will be used interchangeably. These terms include double- and single-stranded DNA, as well as double- and single-stranded RNA, as appropriate for the context.

The term “primer” refers to an oligonucleotide which is capable of acting as a point of initiation of polynucleotide synthesis along a template nucleic acid strand when placed under conditions that permit synthesis of a primer extension product that is complementary to the template strand. The primer can be obtained from a recombinant source, as in a purified restriction fragment, or produced synthetically. Primer extension conditions typically include the presence of four different deoxyribonucleoside triphosphates and an agent with polymerization activity such as DNA polymerase or reverse transcriptase, in a suitable buffer (a “buffer” can include substituents which are cofactors, or which affect pH, ionic strength, etc.), and at a suitable temperature. The primer is preferably single-stranded for maximum efficiency in amplification.

The term “hybridize” refers to binding of a single-stranded nucleic acid or a locally single-stranded region of a double-stranded nucleic acid to another single-stranded nucleic acid or a locally single-stranded region of a double-stranded nucleic acid having a complementary sequence. As one of skill in the art is aware, it is not necessary for two nucleic acid strands to be entirely complementary to hybridize to each other. Depending on the hybridization conditions, a nucleic acid can hybridize to its complement even if there are few, some, or many mismatches, deletions, or additions in one or both strands. In certain embodiments, the primers and probes of the invention can hybridize to an at least partially complementary sequence under stringent conditions, as defined below.

The terms “stringent” or “stringent conditions,” as used herein, denote hybridization conditions of low ionic strength and high temperature, as is well known in the art; see for example Maniatis et al., 1989, Molecular Cloning: A Laboratory Manual, 2d Edition; Current Protocols in Molecular Biology, 1988, ed. Ausubel et al., J. Wiley & Sons publ., New York, and Tijssen, 1993, Techniques in Biochemistry and Molecular Biology-Hybridization with Nucleic Acid Probes, “Overview of principles of hybridization and the strategy of nucleic acid assays,” each of which is hereby incorporated by reference. Generally, stringent conditions are selected to be about 5-30° C. lower than the thermal melting point (Tm) for the specified sequence at a defined ionic strength and pH. Alternatively, stringent conditions are selected to be about 5-15° C. lower than the thermal melting point (Tm) for the specified sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). For example, stringent hybridization conditions can be those in which the salt concentration is less than about 1.0 M sodium (or other salts) ion, typically about 0.01 to about 1 M sodium ion concentration at about pH 7.0 to about pH 8.3 and the temperature is at least about 25° C. for short probes (e.g., 10 to 50 nucleotides) and at least about 55° C. for long probes (e.g., greater than 50 nucleotides). Stringent conditions may also be modified with the addition of hybridization destabilizing agents such as formamide.

The term “highly stringent conditions” is meant to refer to hybridization of a strand of a nucleic acid to a complementary strand of nucleic acid under conditions that permit specific association between the two strands. The nucleic acid strands need not be entirely complementary to hybridize under highly stringent conditions; one of ordinary skill in the art will recognize that nucleic acids can hybridize to each other under highly stringent conditions notwithstanding a certain amount of mismatches, insertions, deletions, etc. One example of “highly stringent conditions” for hybridization of nucleic acids is hybridization in a buffer that comprises 0.5 M NaHPO4, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. 1, at p. 2.10.3). Another example of “highly stringent conditions” that can be used for hybridization of an oligonucleotide to another nucleic acid comprises washing in 6×SSC/0.05% sodium pyrophosphate at 37° C. (for 14 base oligos), 48° C. (for 17 base oligos), 55° C. (for 20 base oligos), and 60° C. (for 23 base oligos).

The term “moderately stringent conditions” is meant to refer to hybridization of a strand of a nucleic acid to a complementary strand of nucleic acid under conditions that permit specific association between the two strands, wherein the strands are less complementary than strands that will associate under highly specific conditions. As one of skill in the art is well aware, “moderately stringent conditions” allow the specific association of nucleic acids that contain sufficient mismatches, insertions, deletions, etc. to prevent specific association under highly stringent conditions, but nonetheless retain sufficient sequence complementarity to specifically associate. One example of “moderately stringent conditions” comprises washing in 0.2×SSC/0.1% SDS at 42° C. (Ausubel et al., 1989, supra).

The “complement” of a nucleic acid sequence, as used herein, refers to an oligonucleotide which, when aligned with the nucleic acid sequence such that the 5′ end of one sequence is paired with the 3 ′ end of the other, is in anti-parallel association. The complement of a nucleic acid sequence need not exactly match every nucleotide of the sequence; stable duplexes may contain mismatched base pairs, unmatched bases, insertions, or deletions. Those skilled in the art of nucleic acid technology can determine duplex stability by empirically considering a number of variables including, for example, the length of the oligonucleotide, base composition and sequence of the oligonucleotide, ionic strength, and incidence of mismatched base pairs.

Stability of a nucleic acid duplex is measured by the melting temperature, or “Tₘ.” The Tₘ of a particular nucleic acid duplex under specified conditions is the temperature at which half of the potential base pairs are disassociated.

The term “detectable moiety” as used herein refers to any atom or molecule which can be used to provide a detectable, quantifiable signal, and which can be attached to a nucleic acid or protein. Detectable moieties may provide signals detectable by fluorescence, radioactivity, colorimetry, gravimetry, X-ray diffraction or absorption, magnetism, enzymatic activity, and the like.

The term “fluorescent moiety” as used herein refers to a chemical moiety that can emit light under conditions appropriate for the particular moiety. Typically, a particular fluorescent moiety can emit light of a particular wavelength following absorbance of light of shorter wavelength. The wavelength of the light emitted by a particular fluorescent moiety is characteristic of that moiety. Thus, a particular fluorescent moiety can be detected by detecting light of an appropriate wavelength following excitation of the fluorescent moiety with light of shorter wavelength. Examples of fluorescent moieties that can be used in the methods and compositions of the present invention include, but are not limited to, fluorescein-family dyes, polyhalofluorescein-family dyes, hexachlorofluorescein-family dyes, coumarin-family dyes, rhodamine-family dyes, cyanine-family dyes, oxazine-family dyes, thiazine-family dyes, squaraine-family dyes, chelated lanthanide-family dyes, and BODIPY®-family dyes.

The term “control assay” as used herein refers to a reaction performed as described below with a compound that has a known effect on regulation of genes associated with hot flash symptoms and/or a known effect on hot flash symptoms. The amount of signal emitted by such a reaction can be compared to a reaction performed using a candidate compound to determine the effect of the candidate compound on regulation of genes associated with hot flash symptoms and/or a known effect on hot flash symptoms.

To determine “percent complementarity” or “percent identity” of two nucleic acid sequences, the sequences are aligned for optimal comparison purposes (e.g., gaps can be introduced in the sequence of a first nucleic acid sequence for optimal alignment with a second nucleic acid sequence). The nucleotides at corresponding nucleotide positions are then compared. When a position in the first sequence is occupied by a complementary nucleotide as the corresponding position in the second sequence, then the molecules are complementary at that position. Likewise, when a position in the first sequence is occupied by the same nucleotide as the corresponding position in the second sequence, then the molecules are identical at that position. The percent complementarity (or percent identity) between the two sequences is a function of the number of complementary positions (or identical positions) shared by the sequences divided by the total number of positions compared (i.e., % complementarity=number of complementary overlapping positions/total number of positions of the shorter nucleotide×100%; and % identity=number of identical overlapping positions/total number of positions of the shorter nucleotide×100%).

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST program of Altschul et al., 1990, J. Mol. Biol. 215:403.

The practice of the present invention will employ, unless otherwise indicated, conventional techniques of molecular biology, microbiology and recombinant DNA techniques, which are within the skill of the art. Such techniques are explained fully in the literature. See, e.g., Sambrook et al., 2001, Molecular Cloning: A Laboratory Manual, Third Edition, Cold Spring Harbor Laboratory Press, Cold Spring Harbor, New York; Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.).

### 6.2. Methods of Determining the Effect of a Candidate Compound on Hot Flash Symptoms

The present invention provides a method for determining the effect of a candidate compound on hot flash symptoms. The method generally comprises exposing a cell that expresses an estrogen receptor or estrogen related receptor to a candidate compound and determining the effect of the candidate compound on the cell's expression of a panel of genes associated with hot flash symptoms. In certain embodiments, the method can be used to identify the effect of a single compound on the expression of a panel of genes associated with hot flash symptoms. In other embodiments, the methods can identify the effect of more than one compound on the expression of a panel of genes associated with hot flash symptoms. Thus, the method of the invention can be used to achieve at least two major objectives: the method can be used to identify compounds that decrease the incidence of hot flash symptoms, and the method can be used to determine whether compounds that have some other, useful effect, e.g., compounds effective to treat osteoporosis or breast cancer, undesirably increase the incidence of hot flash symptoms.

### 6.2.1. Compounds Having a Known Effect on Hot Flash Symptoms

The present invention is based, in part, on the discovery of a correlation between regulation of expression of certain genes in a cell that expresses the estrogen receptor or estrogen related receptor and contact of the cell by compounds that have a known effect on hot flash symptoms. Thus, based upon the disclosure of the present invention, one of skill in the art can determine the effect of a candidate compound on hot flash symptoms by contacting a cell with the candidate compound, and determining the effect of the candidate compound on the expression of genes associated with hot flash symptoms. The effects of the candidate compound on hot flash symptoms can be determined by comparing the expression of the panel of genes with a reference expression profile generated by a compound or compounds that has a known effect on hot flash symptoms.

Any compound, without limitation, that is known by one of skill in the art to have an effect on hot flash symptoms when administered to a subject can be used as a reference compound in the methods of the invention. The reference compound can either increase or decrease the incidence of hot flash symptoms. Gene expression in a cell that expresses the estrogen receptor or estrogen related receptor can be assessed in the presence and absence of the reference compound. Genes that are differentially expressed following contact with a reference compound can thus be identified. The expression of such genes can be upregulated or downregulated following such contact with a reference compound.

Following identification of genes that are upregulated or downregulated by compounds that have a known effect on hot flash symptoms, the effects of the reference compound on gene expression and on hot flash symptoms can be correlated. For example, estradiol and tibolone are known to decrease the incidence of hot flash symptoms when administered to a subject, while raloxifene and 4-hydroxy tamoxifen are known to increase the incidence of hot flash symptoms when administered to a subject. Thus, a candidate compound that has a similar effect on gene expression to estradiol or tibolone is likely to decrease the incidence of hot flash symptoms when administered to a subject. Conversely, a candidate compound that has a similar effect on gene expression to raloxifene or 4-hydroxy tamoxifen is likely to decrease the incidence of hot flash symptoms when administered to a subject. Any other compound whose effect on hot flash symptoms is known to one of skill in the art can be similarly used in the methods of the invention.

Accordingly, in certain aspects, the present invention provides methods of determining the effect of a candidate compound or a library of candidate compounds on hot flash symptoms. The methods generally comprise determining the effect of the candidate compound on expression of a panel of genes associated with hot flash symptoms.

In certain embodiments, the methods further comprise comparing the expression of the panel of genes associated with hot flash symptoms with a reference expression profile of the panel of genes associated with hot flash symptoms. In certain embodiments, the reference expression profile of the panel of genes associated with hot flash symptoms can be the expression profile of the panel of genes following contact of a cell with a compound selected from the group consisting of estradiol, tibolone, raloxifene, and 4-hydroxy tamoxifen. In other embodiments, the reference expression profile of the panel of genes associated with hot flash symptoms can be the expression profile of the panel of genes following contact of a cell with estradiol. In still other embodiments, the reference expression profile of the panel of genes associated with hot flash symptoms can be the expression profile of the panel of genes following contact of a cell with tibolone. In yet other embodiments, the reference expression profile of the panel of genes associated with hot flash symptoms can be the expression profile of the panel of genes following contact of a cell with raloxifene. In still other embodiments, the reference expression profile of the panel of genes associated with hot flash symptoms can be the expression profile of the panel of genes following contact of a cell with 4-hydroxy tamoxifen.

In other embodiments, the method further comprises comparing the expression profile of a panel of genes associated with hot flash symptoms in a cell following contact with a candidate compound with the expression profile of the panel of genes associated with hot flash symptoms in a cell following contact with a reference compound. In certain embodiments, the reference compound can be selected from the group that consists of estradiol, tibolone, raloxifene, and 4-hydroxy tamoxifen. In other embodiments, the reference compound can be estradiol. In still other embodiments, the reference compound can be tibolone. In yet other embodiments, the reference compound can be raloxifene. In still other embodiments, the reference compound can be 4-hydroxy tamoxifen.

### 6.2.2. Genes Associated with Hot Flash Symptoms and Their Regulation By Compounds Having a Known Effect on Hot Flash Symptoms

The present invention provides a large number of genes that are differentially expressed following contact with a compound that has a known effect on hot flash symptoms. The effect on expression of such genes in a cell following contact with a candidate compound can be compared to the effect on expression of these genes following contact with a reference compound, thereby determining the effect of the candidate compound on hot flash symptoms. Genes that can are differentially expressed following contact with a compound known to affect hot flash symptoms are described below.

The expression of any gene known by one of skill in the art to be associated with hot flash symptoms, without limitation, can be assessed in the methods of the invention. The present invention provides numerous such genes as described in detail, below. Further, additional genes that are associated with hot flash symptoms can readily be identified by one of skill in the art according to the disclosure of the present invention. For example, a cell that expresses the estrogen receptor or estrogen related receptor can be contacted with a reference compound known to affect hot flash symptoms. The overall expression of genes in the cell following contact with the reference compound can be compared to the overall expression of genes in a similar cell that has not been contacted with the reference compound.

The expression of such genes to identify genes that are differentially expressed following contact with a compound with a known effect on hot flash symptoms can be monitored by any convenient method known by one of skill in the art. Most advantageously, the expression of many genes, e.g., thousands of genes, is monitored at once using a high throughput method for assessing expression of genes in the presence and absence of compounds known to affect hot flash symptoms.

Using, for example, a gene array from Affymetrix, Inc. (Sunnyvale, Calif.), genes that are differentially expressed following contact with the reference compound can be identified. Affymetrix gene arrays, and methods of making and using such arrays, are described in, for example, U.S. Pat. Nos. 6,551,784, 6,548,257, 6,505,125, 6,489,114, 6,451,536, 6,410,229, 6,391,550, 6,379,895, 6,355,432, 6,342,355, 6,333,155, 6,308,170, 6,291,183, 6,287,850, 6,261,776, 6,225,625, 6,197,506, 6,168,948, 6,156,501, 6,141,096, 6,040,138, 6,022,963, 5,919,523, 5,837,832, 5,744,305, 5,834,758, and 5,631,734, each of which is hereby incorporated by reference in its entirety. In addition, Ausubel et al., eds., Current Protocols in Molecular Biology, 2002, Vol. 4, Unit 25B, Ch. 22, which is hereby incorporated by reference in its entirety, provides further guidance on construction and use of a gene array for identifying genes of interest, e.g., genes associated with hot flash symptoms, that are differentially expressed in different samples of cells.

Other techniques suitable for such analysis include differential PCR, restriction mediated differential display, AFLP-based transcript profiling, serial expression of gene analysis (“SAGE”), and Massive Parallel Signature Sequencing (“MPSS”). These other techniques and protocols for performing the techniques are well-known in the art and are described in Ausubel et al., eds., Current Protocols in Molecular Biology, 2002, Vol. 4, Unit 25B, Ch. 3-6, except for MPSS, which is described in Brenner et al., 2000, Nat. Biochem. 18:630-634. Each of these references is hereby incorporated by reference in its entirety. Expression of genes that are identified in such a manner can be assessed to determine the effect of candidate compounds on hot flash symptoms.

Using the above-described methods, a number of genes have been identified that are associated with hot flash symptoms. TABLE 1, below, presents a number of examples of such genes and further, provides examples of effects on regulation of expression of these genes in a cell following contact with reference compounds that have known effects on hot flash symptoms.

TABLE 1

| | Abbreviation | | | | | Effect of 4- |
|---|---|---|---|---|---|---|
| | or | | Effect of | Effect of | Effect of | hydroxy |
| | Alternative | Accession | Estradiol on | Tibolone on | Raloxifene on | tamoxifen on |
| Gene Name | Name | Number | Expression | Expression | Expression | Expression |
| Activin Beta E | | AF089825 | Upregulated | Upregulated | Down- | Down- |
| SEQ ID NO: 1 | | | | | regulated | regulated |
| Type II | HKII | D26393 | Upregulated | Upregulated | Down- | Down- |
| Hexokinase | | | | | regulated | regulated |
| SEQ ID NO: 2 | | | | | | |
| Multi Drug | MDR | M81855 | Upregulated | Upregulated | Down- | Down- |
| Resistance Gene | | | | | regulated | regulated |
| SEQ ID NO: 3 | | | | | | |
| Parvalbumin | | AF022935 | Upregulated | Upregulated | Down- | Down- |
| SEQ ID NO: 4 | | | | | regulated | regulated |
| Protein Tyrosine | BAD2 | U02553 | Upregulated | Upregulated | Down- | Down- |
| Phosphatase | | | | | regulated | regulated |
| BAD2 | | | | | | |
| SEQ ID NO: 5 | | | | | | |
| Prolactin | | AI175539 | Upregulated | Upregulated | Down- | Down- |
| SEQ ID NO: 6 | | | | | regulated | regulated |
| Argininosuccinate | | X12459 | Upregulated | Upregulated | Down- | Down- |
| Synthetase | | | | | regulated | regulated |
| SEQ ID NO: 7 | | | | | | |
| Ribonucleoside | RNR-1 | L08595 | Upregulated | Upregulated | No Significant | No Significant |
| Reductase | | | | | Effect | Effect |
| Nuclear Receptor | | | | | | |
| SEQ ID NO: 8 | | | | | | |
| Interleukin-18 | IL-18 | AJ222813 | Upregulated | Upregulated | No Significant | No Significant |
| SEQ ID NO: 9 | | | | | Effect | Effect |
| ARL gene 4 | | X77235 | Upregulated | Upregulated | No Significant | No Significant |
| SEQ ID NO: 10 | | | | | Effect | Effect |
| Calpain | | D14478 | Upregulated | Upregulated | No Significant | No Significant |
| SEQ ID NO: 11 | | | | | Effect | Effect |
| EST196325 | | AA892522 | Upregulated | Upregulated | No Significant | No Significant |
| SEQ ID NO: 12 | | | | | Effect | Effect |
| Interleukin-1 | CPP32 | U84410 | Upregulated | Upregulated | No Significant | No Significant |
| β-converting | | | | | Effect | Effect |
| enzyme-related | | | | | | |
| protease CPP32 | | | | | | |
| SEQ ID NO: 13 | | | | | | |
| EST208064 | | AI013389 | Upregulated | Upregulated | No Significant | No Significant |
| SEQ ID NO: 14 | | | | | Effect | Effect |
| Amiloride | | X73911 | Down- | Down- | No Significant | No Significant |
| Binding Protein | | | regulated | regulated | Effect | Effect |
| SEQ ID NO: 15 | | | | | | |
| 2-alpha-1 globin | | X56325 | Upregulated | Upregulated | No Significant | No Significant |
| SEQ ID NO: 16 | | | | | Effect | Effect |
| Annexin 1 | EST 21795 | AI171962 | No | No | Upregulated | Upregulated |
| SEQ ID NO: 17 | | | Significant | Significant | | |
| | | | Effect | Effect | | |
| N27 | EST 207724 | AI014169 | No | No | Upregulated | Upregulated |
| SEQ ID NO: 18 | | | Significant | Significant | | |
| | | | Effect | Effect | | |
| HMG-box | HBP1 | U09551 | No | No | Upregulated | Upregulated |
| containing protein 1 | | | Significant | Significant | | |
| SEQ ID NO: 19 | | | Effect | Effect | | |
| D-binding protein | | J03179 | No | No | Upregulated | Upregulated |
| SEQ ID NO: 20 | | | Significant | Significant | | |
| | | | Effect | Effect | | |
| FE65 adaptor | FE65 | X60469 | No | No | Upregulated | No Significant |
| protein | | | Significant | Significant | | Effect |
| interacting with | | | Effect | Effect | | |
| beta-amyloid | | | | | | |
| precursor protein | | | | | | |
| intracellular | | | | | | |
| domain | | | | | | |
| SEQ ID NO: 21 | | | | | | |
| Protein Kinase C | PKC type I | M13707 | No | No | Upregulated | Upregulated |
| type I | | | Significant | Significant | | |
| SEQ ID NO: 22 | | | Effect | Effect | | |
| Glutamate | | U08255 | No | No | Down- | No Significant |
| Receptor (d1 | | | Significant | Significant | regulated | Effect |
| subunit) | | | Effect | Effect | | |
| SEQ ID NO: 23 | | | | | | |
| Vesicle | VAP1 | AF034582 | No | No | Down- | No Significant |
| Associated | | | Significant | Significant | regulated | Effect |
| Protein VAP1 | | | Effect | Effect | | |
| SEQ ID NO: 24 | | | | | | |
| Protein Kinase C | | M18331 | No | No | Down- | No Significant |
| subspecies | | | Significant | Significant | regulated | Effect |
| epsilon | | | Effect | Effect | | |
| SEQ ID NO: 25 | | | | | | |
| EST 203549 | | AI009098 | No | No | Down- | No Significant |
| SEQ ID NO: 26 | | | Significant | Significant | regulated | Effect |
| | | | Effect | Effect | | |
| Heat Shock | | X83094 | No | No | Down- | No Significant |
| Transcription | | | Significant | Significant | regulated | Effect |
| Factor 1 | | | Effect | Effect | | |
| SEQ ID NO: 27 | | | | | | |

As shown in TABLE 1, the present invention provides a number of genes that are differentially expressed following contact with compounds that have known effects on hot flash symptoms. By comparing the effects of the reference compounds on expression of genes associated with hot flash symptoms to the effect of a candidate compound on expression of these genes, the effect of the candidate compound on hot flash symptoms can be determined.

Accordingly, in certain aspects, the invention provides methods of determining the effect of a candidate compound on hot flash symptoms that comprise determining the effect of the candidate compound on expression of a panel of genes associated with hot flash symptoms.

In certain embodiments, at least one member of the panel of genes that is associated with hot flash symptoms can be selected from the group consisting of Activin Beta E, Type II Hexokinase, Multi Drug Resistance Gene, Parvalbumin, BAD2, Prolactin, Argininosuccinate Synthetase, Ribonucleoside Reductase 1, Interleukin-18, ARL gene 4, Calpain, EST196325, CPP32, EST208064, 2-alpha-1 globin, Amiloride Binding Protein, Annexin 1, N27, HBP1, D-binding protein, FE65, Protein Kinase C type I, Glutamate Receptor subunit d1, VAP1, Protein Kinase C subspecies epsilon, EST203549, and Heat Shock Transcription Factor 1. In other embodiments, more than one member of the panel of genes that is associated with hot flash symptoms can be selected from the group consisting of Activin Beta E, Type II Hexokinase, Multi Drug Resistance Gene, Parvalbumin, BAD2, Prolactin, Argininosuccinate Synthetase, Ribonucleoside Reductase 1, Interleukin-18, ARL gene 4, Calpain, EST196325, CPP32, EST208064, 2-alpha-1 globin, Amiloride Binding Protein, Annexin 1, N27, HBP1, D-binding protein, FE65, Protein Kinase C type I, Glutamate Receptor subunit d1, VAP1, Protein Kinase C subspecies epsilon, EST203549, and Heat Shock Transcription Factor 1. In still other embodiments, each member of the panel of genes that is associated with hot flash symptoms can be selected from the group consisting of Activin Beta E, Type II Hexokinase, Multi Drug Resistance Gene, Parvalbumin, BAD2, Prolactin, Argininosuccinate Synthetase, Ribonucleoside Reductase 1, Interleukin-18, ARL gene 4, Calpain, EST196325, CPP32, EST208064, 2-alpha-1 globin, Amiloride Binding Protein, Annexin 1, N27, HBP1, D-binding protein, FE65, Protein Kinase C type I, Glutamate Receptor subunit d1, VAP1, Protein Kinase C subspecies epsilon, EST203549, and Heat Shock Transcription Factor 1.

In more preferred embodiments, at least one member of the panel of genes associated from hot flash symptoms can be selected from the group consisting of Type II Hexokinase, Multi Drug Resistance Gene, Parvalbumin, BAD2, Interleukin-18, Calpain, EST196325, Annexin 1, N27, HBP1, and Protein Kinase C subspecies epsilon. In still more preferred embodiments, more than one member of the panel of genes associated from hot flash symptoms can be elected from the group consisting of Type II Hexokinase, Multi Drug Resistance Gene, Parvalbumin, BAD2, Interleukin-18, Calpain, EST196325, Annexin 1, N27, HBP1, and Protein Kinase C subspecies epsilon. In yet more preferred embodiments, each member of the panel of genes associated from hot flash symptoms can be selected from the group consisting of Type II Hexokinase, Multi Drug Resistance Gene, Parvalbumin, BAD2, Interleukin-18, Calpain, EST196325, Annexin 1, N27, HBP1, and Protein Kinase C subspecies epsilon.

In certain embodiments, the panel of genes comprises two genes associated with hot flash symptoms. In other, more preferred, embodiments, the panel of genes comprises four genes associated with hot flash symptoms. In other, even more preferred, embodiments, the panel of genes comprises five genes associated with hot flash symptoms. In yet other, even more preferred, embodiments, the panel of genes comprises eight genes associated with hot flash symptoms. In the most preferred embodiment, the panel of genes comprises ten genes associated with hot flash symptoms.

The expression of the members of the panel of genes associated with hot flash symptoms can be either upregulated or downregulated. As shown above in TABLE 1, reference compounds can characteristically increase or decrease the expression of genes associated with hot flash symptoms. Thus, the nature of the regulation of expression, e.g., upregulation or downregulation, of genes associated with hot flash symptoms can also be used to determine the effect of a candidate compound on hot flash symptoms. For example, contacting a cell that expresses the estrogen receptor with estradiol results in the upregulation of BAD2 and HKII, while contact of the cell with 4-hydroxy tamoxifen results in the downregulation of these genes.

Accordingly, in certain embodiments, expression of at least one member of the panel of genes associated with hot flash symptoms can be upregulated in a cell that expresses the estrogen receptor or estrogen related receptor following contact with a candidate compound. In certain embodiments, expression of at least one member of the panel of genes associated with hot flash symptoms can be not upregulated in a cell that expresses the estrogen receptor or estrogen related receptor following contact with a candidate compound. In certain embodiments, expression of at least one member of the panel of genes associated with hot flash symptoms can be downregulated in a cell that expresses the estrogen receptor or estrogen related receptor following contact with a candidate compound. In certain embodiments, expression of at least one member of the panel of genes associated with hot flash symptoms can be not downregulated in a cell that expresses the estrogen receptor or estrogen related receptor following contact with a candidate compound.

In certain embodiments, expression of Type II Hexokinase, Multi Drug Resistance Gene, Parvalbumin, BAD2, Prolactin, Interleukin-18, Calpain, or EST196325 can be upregulated in a cell that expresses the estrogen receptor or estrogen related receptor following contact with the candidate compound. In certain embodiments, expression of Annexin 1, N27, or HBP1 can be not upregulated in a cell that expresses the estrogen receptor or estrogen related receptor following contact with the candidate compound. In certain embodiments, expression of Protein Kinase C subspecies epsilon can be not downregulated in a cell that expresses the estrogen receptor or estrogen related receptor following contact with the candidate compound.

In other embodiments, expression of Type II Hexokinase, Multi Drug Resistance Gene, Parvalbumin, BAD2, Prolactin, Interleukin-18, Calpain, or EST196325 can be not upregulated in a cell that expresses the estrogen receptor or estrogen related receptor following contact with the candidate compound. In certain embodiments, expression of Annexin 1, N27, or HBP1 can be upregulated in a cell that expresses the estrogen receptor or estrogen related receptor following contact with the candidate compound. In certain embodiments, expression of Protein Kinase C subspecies epsilon can be downregulated in a cell that expresses the estrogen receptor or estrogen related receptor following contact with the candidate compound.

In a preferred embodiment, expression of Type II Hexokinase, Multi Drug Resistance Gene, Parvalbumin, BAD2, Prolactin, Interleukin-18, Calpain, and EST196325 are upregulated in a cell that expresses the estrogen receptor or estrogen related receptor following contact with the candidate compound; expression of Annexin 1, N27, and HBP1 is not upregulated in a cell that expresses the estrogen receptor or estrogen related receptor following contact with the candidate compound; and expression of Protein Kinase C subspecies epsilon is not downregulated in a cell that expresses the estrogen receptor or estrogen related receptor following contact with the candidate compound.

The amount of the change in expression of a panel of genes associated with hot flash symptoms in a cell following contact with a candidate compound can also be assayed in order to determine the effects of the candidate compound on hot flash symptoms. A greater change in expression of genes associated with hot flash symptoms correlates with a more potent effect on hot flash symptoms. Further, the direction of regulation, either up or down, can be compared to the effect of the compounds with a known effect on hot flash symptoms.

For example, estradiol is the most potent therapeutic agent known to decrease the incidence of hot flash symptoms. If a candidate compound causes the expression of a gene that is upregulated following contact with estradiol, for example, HKII, to increase a similar or greater degree as estradiol, then the candidate compound can be determined to decrease the incidence of hot flash symptoms. The same relationship applies to the effects of tibolone, raloxifene, and 4-hydroxy tamoxifen on regulation of expression of genes associated with hot flash symptoms and the determined effect of a candidate compound on hot flash symptoms. However, even if a candidate compound causes the expression of a gene, for example, HKII, to increase, but to a lesser degree than estradiol, the candidate compound may still decrease the incidence of hot flash symptoms.

In such situations, the relative amount of increase or decrease in expression of a gene associated with hot flash symptoms following contact with a candidate compound can be determined. For example, a candidate compound may cause the expression of a gene, for example HKII, to increase about two-fold, i.e., by about 100%. Notwithstanding the relative increase or decrease in expression of a given gene following contact with estradiol or tibolone, a candidate compound that results in about a two-fold increase in expression of a gene that is upregulated following contact with estradiol or tibolone is likely to decrease the incidence of hot flash symptoms.

Similarly, a candidate compound that results in an about two-fold decrease, i.e., by about 50%, in expression of a gene that is downregulated following contact with estradiol or tibolone is likely to decrease the incidence of hot flash symptoms. The converse is also true; a candidate compound that results in an about two-fold increase, i.e., by about 100%, in expression of a gene that is upregulated following contact with raloxifene or 4-hydroxy tamoxifen is likely to increase the incidence of hot flash symptoms. Similarly, a candidate compound that results in an about two-fold decrease, i.e., by about 50%, in expression of a gene that is downregulated following contact with raloxifene or 4-hydroxy tamoxifen is likely to increase the incidence of hot flash symptoms.

### 6.2.3. Determining the Effect of a Candidate Compound on Hot Flash Symptoms

The present invention provides methods of determining the effect of a candidate compound on hot flash symptoms based, in part, on the discovery of a correlation between regulation of a panel of genes associated with hot flash symptoms in a cell and contact with the cell by a reference compound with a known effect on hot flash symptoms. The genes associated with hot flash symptoms, reference compounds with known effects on hot flash symptoms, and the effects of such compounds on regulation of such genes are described extensively above. The effects of candidate compounds on hot flash symptoms can be determined by observing the effects of such compounds on the regulation of a panel of genes associated with hot flash symptoms.

Generally, the methods of the invention provide for exposing a cell that expresses the estrogen receptor or estrogen related receptor to a candidate compound, and determining the effect of the candidate compound on the regulation of one or more genes that are associated with hot flash symptoms. Preferably, the effects of the candidate compound on regulation of more than two, four, five, six, or eight genes that are associated with hot flash symptoms are determined. Most preferably, the effects of the candidate compound on regulation of about ten genes that are associated with hot flash symptoms are determined.

In certain embodiments, the effect of a candidate compound on the regulation of genes associated with hot flash symptoms can correlate with the effect of a compound that is selected from the group of estradiol, tibolone, raloxifene, and 4-hydroxy tamoxifen. In other embodiments, the effect of a candidate compound on the regulation of genes associated with hot flash symptoms can correlate with the effect of a compound that is selected from the group of estradiol and tibolone. In still other embodiments, the effect of a candidate compound on the regulation of genes associated with hot flash symptoms can correlate with the effect of a compound that is selected from the group of raloxifene and 4-hydroxy tamoxifen. In yet other embodiments, the effect of a candidate compound on the regulation of genes associated with hot flash symptoms can correlate with the effect of estradiol. In still other embodiments, the effect of a candidate compound on the regulation of genes associated with hot flash symptoms can correlate with the effect of tibolone. In yet other embodiments, the effect of a candidate compound on the regulation of genes associated with hot flash symptoms can correlate with the effect of raloxifene. In still other embodiments, the effect of a candidate compound on the regulation of genes associated with hot flash symptoms can correlate with the effect of 4-hydroxy tamoxifen.

In certain embodiments, the effect of the candidate compound on the regulation of a panel genes associated with hot flash symptoms can be different from the effect of a compound that is selected from the group consisting of estradiol, tibolone, raloxifene, and 4-hydroxy tamoxifen. Nonetheless, one of skill in the art can still determine the effect of the candidate compound on hot flash symptoms based upon the degree of similarity of effect on hot flash symptoms of the candidate compound to the effects of the reference compounds.

In general, the effect of the candidate compound on regulation of genes associated with hot flash symptoms, particularly the direction of regulation, can be compared to the effects of the reference compounds on regulation of the same genes. If the candidate compound regulates the expression of at least about half of the genes associated with hot flash symptoms that are tested similar to the manner in which estradiol or tibolone regulate the same genes, but no such genes are regulated similar to the manner in which raloxifene or 4-hydroxy tamoxifen regulate the same genes, the candidate compound can be determined to decrease the incidence of hot flash symptoms. If the candidate compound regulates the expression of at least about half of genes associated with hot flash symptoms that are tested similar to the manner in which raloxifene or 4-hydroxy tamoxifen regulate the same genes, but no such genes are regulated similar to the manner in which estradiol or tibolone regulate the same genes, the candidate compound can be determined to increase the incidence of hot flash symptoms. Preferably, expression of at least ten such genes is tested in determining the effect of the candidate compound, as shown in the following examples. However, as few as five genes associated with hot flash symptoms can be tested in determining the effect of a candidate compound hot flash symptoms.

For example, a candidate compound could increase the expression of at least about five genes associated with hot flash symptoms that are upregulated in a cell following contact with estradiol and/or tibolone, but not increase the expression of any genes that are upregulated in a cell following contact with raloxifene and/or 4-hydroxy tamoxifen. Such a candidate compound would be determined to decrease the incidence of hot flash symptoms. In another example, a candidate compound that can decrease the expression of at least about five genes associated with hot flash symptoms that are downregulated in a cell following contact with estradiol and/or tibolone, but not increase the expression of any genes that are upregulated in a cell following contact with raloxifene and/or 4-hydroxy tamoxifen would also be determined to decrease the incidence of hot flash symptoms. Other examples of candidate compounds that would be determined to decrease the incidence of hot flash symptoms include those that increase the expression of at least about five genes associated with hot flash symptoms that are upregulated in a cell following contact with estradiol and/or tibolone, but not decrease the expression of any genes that are downregulated in a cell following contact with raloxifene and/or 4-hydroxy tamoxifen and those that can decrease the expression of at least about five genes associated with hot flash symptoms that are downregulated in a cell following contact with estradiol and/or tibolone, but not decrease the expression of any genes that are downregulated in a cell following contact with raloxifene and/or 4-hydroxy tamoxifen.

In another example, a compound that can increase the expression of at least about five genes that are upregulated in a cell following contact with raloxifene or 4-hydroxy tamoxifen but does not increase the expression of any genes that are upregulated in a cell following contact with estradiol or tibolone would be determined to increase the incidence of hot flash symptoms. In still another example, candidate compounds that decrease the expression of at least about five genes that are downregulated in a cell following contact with raloxifene and 4-hydroxy tamoxifen but do not decrease the expression of any genes that are downregulated in a cell following contact with estradiol or tibolone would be determined to increase the incidence of hot flash symptoms. Other examples of candidate compounds that would be determined to increase the incidence of hot flash symptoms include those that that can increase the expression of at least about five genes that are upregulated in a cell following contact with raloxifene or 4-hydroxy tamoxifen but does not decrease the expression of any genes that are downregulated in a cell following contact with estradiol or tibolone and those that decrease the expression of at least about five genes that are downregulated in a cell following contact with raloxifene and 4-hydroxy tamoxifen but do not increase the expression of any genes that are upregulated in a cell following contact with estradiol or tibolone.

In certain embodiments, the effect of the candidate compound on regulation of genes associated with hot flash symptoms can be compared to a reference expression profile of the panel of genes associated with hot flash symptoms. The reference expression profile of the panel of genes associated with hot flash symptoms is generally determined by contacting a cell with a compound selected from the group consisting of estradiol, tibolone, raloxifene, and 4-hydroxy tamoxifen. The reference pattern can be predetermined, i.e., known at the time the candidate compound is contacted to the cell to determine the effect of the candidate compound on regulation of hot flash symptoms. The reference pattern can comprise any set of one or more genes and effects on hot flash symptoms listed in TABLE 1.

In other embodiments, the methods further comprise comparing a cell's expression of the panel of genes associated with hot flash symptoms following contact with a candidate compound with a cell's expression of the panel of genes associated with hot flash symptoms following contact with a compound that has a known effect on hot flash symptoms. The compound that has a known effect on hot flash symptoms can be any such compound known to one of skill in the art without limitations. In certain embodiments, the compound that has a known effect on hot flash symptoms can be selected from the group that consists of estradiol, tibolone, raloxifene, and 4-hydroxy tamoxifen. In such embodiments, the compound or compounds with known effects on hot flash symptoms are contacted with a cell in parallel with one or more candidate compounds as a control assay. The effects of the candidate compounds and reference compounds on regulation of expression of genes associated with hot flash symptoms can be compared to determine the effect of the candidate compound on hot flash symptoms.

### 6.2.4. Cells and Cell Lines that Express the Estrogen Receptor and Estrogen Related Receptor

The present invention is based, in part, on assessing the regulation of genes associated with hot flash symptoms in cells that express the estrogen receptor or estrogen related receptor following contact with compounds that can modulate the estrogen receptor or estrogen related receptor. Accordingly, certain aspects of the invention rely on contacting a cell that expresses the estrogen receptor or estrogen related receptor with a candidate compound.

Any cell that is known by one of skill in the art to express the estrogen receptor or estrogen related receptor, without limitation, may be used in the methods of the invention. In certain embodiments, the cell can express the estrogen receptor. In other embodiments, the cell can express the estrogen related receptor. Such cells that express one of these receptors are generally mammalian cells. In certain embodiments, the mammalian cell can be selected from the group that consists of a rat cell, a mouse cell, a monkey cell, a chimpanzee cell, and a human cell.

Further, the cell can be derived from any organ or tissue that expresses the estrogen receptor or the estrogen related receptor. For example, in certain embodiments, the cell that expresses the estrogen receptor can be a pituitary cell. In other embodiments, the cell that expresses the estrogen receptor can be a hypothalamus cell. In still other embodiments, the cell that expresses the estrogen related receptor can be any central or peripheral nervous system cell that expresses the estrogen related receptor.

The cell that expresses the estrogen receptor or estrogen related receptor and that is used in the methods of the invention is preferably a cell suitable for propagation in cell culture for an indefinite period of time. Any such cell that is known by one of skill in the art to express the estrogen receptor or estrogen related receptor and to be suitable for propagation in cell culture for an indefinite period of time can be used in the methods of the invention, without limitation. In certain embodiments, the cell that expresses the estrogen receptor can be selected from the group of a GH3 cell, a GH4 cell, a PR1 cell, a MtT/E-2 cell, a alphaT3-1 cell, a D12 cell, an RCF-8 cell, and a GT1-7 cell. In a preferred embodiment, the cell that expresses the estrogen receptor is a GH3 cell. In certain embodiments, the cell that expresses the estrogen related receptor can be selected from the group that consists of an A172 glioma cell, a MCF10a cell, a MCF12 cell, a MDA-MB-231 cell, a MDA-MB-435 cell, a MDA-MB-436 cell, a MDA-MB-468 cell, a Hs 578T cell, a BT 20 cell, a BT 474 cell, a BT 549 cell, a SKBr 3 cell, a ZR 75.1 cell, a T47D cell, and a MCF7 cell.

Further, the cell that expresses the estrogen receptor can express any estrogen receptor known by one of skill in the art without limitation. In certain embodiments, the estrogen receptor that is expressed by the cell can be estrogen receptor α. In other embodiments, the estrogen receptor that is expressed by the cell can be estrogen receptor β. In still other embodiments, the cell that expresses the estrogen receptor can express both estrogen receptor a and estrogen receptor β.

Similarly, the cell that expresses the estrogen related receptor can express any estrogen related receptor known to one of skill in the art without limitation. In certain embodiments, the cell that expresses the estrogen related receptor can express estrogen related receptor α. In other embodiments, the cell that expresses the estrogen related receptor can express estrogen related receptor β. In yet other embodiments, the cell that expresses the estrogen related receptor can express estrogen related receptor γ. In still other embodiments, the cell that expresses the estrogen related receptor can express two or three estrogen related receptors, each of which is selected from the group that consists of estrogen related receptor α, estrogen related receptor β, and estrogen related receptor γ.

### 6.2.5. High Throughput Methods for Ouantifying the Expression of Genes Associated with Hot Flash Symptoms

In certain aspects, the present invention provides high throughput methods for determining the effect of a plurality of candidate compounds on hot flash symptoms. High throughput methods for determining the effect of a plurality of compounds on hot flash symptoms generally comprise separately exposing samples of cells that express the estrogen receptor or estrogen related receptor to each member of the plurality of candidate compounds. The effects of each of the plurality of candidate compounds on the regulation of expression of a panel of genes associated with hot flash symptoms can then be determined for each of the samples of cells. Using the above-described correlation between regulation of expression of such genes and the effect of a reference compound on hot flash symptoms, the effects of the plurality of compounds can thus be identified.

High throughput methods generally rely on simultaneously determining the effects of a large number of compounds on the regulation of expression of a panel of genes associated with hot flash symptoms in a sample of cells. The high throughput methods used in connection with the invention are further described with reference to particular embodiments of such high throughput methods as described below. Further guidance regarding these particular embodiments of the high throughput methods for determining the effect of candidate compounds on hot flash symptoms may be found in U.S. Pat. No. 6,238,869, which is incorporated by reference in its entirety. This patent describes generic methods that can be readily be adapted to determine the effect of candidate compound on regulation of expression of genes associated with hot flash symptoms as described below. However, any high throughput method known to one of skill in the art to be useful in the determination of the effects of a large number of compounds on regulation of gene expression, without limitation, can be used in the methods of the invention.

Briefly, the preferred high throughput method comprises separately exposing samples of cells to each member of a plurality of candidate compounds. The samples of cells are then lysed. The lysates are exposed to a plurality of gene-specific nuclease protection probes, each of which can hybridize to a member of a panel of genes associated with hot flash symptoms, as described above. The lysates are then contacted with a nuclease with single-stranded nuclease activity, such as, for example, S1 nuclease. Following digestion to completion with this nuclease, the remaining RNA in the lysates can optionally be degraded.

The remainder of the method comprises detecting the gene specific nuclease protection probes or RNA protected from degradation by the gene-specific nuclease protection probes. In one embodiment, the lysates are contacted with a surface containing regions adapted to specifically associate with each of the gene-specific nuclease protection probes. The presence and amount of each gene-specific nuclease protection probe can be detected by contacting the gene-specific nuclease protection probes with a detection probe that specifically associates with the gene-specific nuclease protection probes. The detection probe can then be detected directly or indirectly.

In addition, certain other methods for quantifying the expression of genes associated with hot flash symptoms, as described in Section 5.2.6, below, can be adapted by one of skill in the art to identify the effects of a large number of candidate compounds and thus can be used as high throughput methods. Therefore, the high throughput methods for identifying the effects of candidate compounds on hot flash symptoms are not limited to the above-described embodiments, but also include those described below as well. Further, the methods for identifying genes associated with hot flash symptoms can also be adapted to determine the effects of candidate compounds on regulation of expression of genes associated with hot flash symptoms and thus can be used to determine the effects of such candidate compounds on hot flash symptoms.

### 6.2.6. Other Methods for Quantifying the Expression of Genes Associated with Hot Flash Symptoms

In certain aspects, the invention provides methods for determining the effect of a candidate compound on hot flash symptoms by determining the effect of the candidate compound on expression of a panel of genes associated with hot flash symptoms. Generally, the effect of the candidate compound on expression of genes associated with hot flash symptoms can be determined by quantifying the amount of mRNA expressed from a gene associated with hot flash symptoms by a cell in the presence and absence of the candidate compound. By comparing the amount of mRNA expressed from a gene associated with hot flash symptoms by a cell that has been exposed to a candidate compound to the amount of mRNA expressed from a gene associated with hot flash symptoms by a cell that has not been exposed to the candidate compound, the effect of the candidate compound on expression of genes associated with hot flash symptoms can be determined.

Any method known by one of skill in the art to be useful in detecting the presence and amount of an mRNA expressed from a gene associated with hot flash symptoms can be used in the methods of the invention, without limitation. For example, the presence and amount of an mRNA expressed from a gene associated with hot flash symptoms can be detected with a technique that is selected from the group consisting of reverse transcription real time PCR, quantitative reverse transcription PCR, Northern blot assays, dot blot assays, reverse dot blot assays, S1 nuclease protection assays, primer extension, RNAse protection assays, 5′-nuclease assays, reporter gene assays, branched DNA assays, bead array assays, and multiplexed array mRNA assays. In a preferred embodiment, the presence and amount of an mRNA expressed from a gene associated with hot flash symptoms is detected with a multiplexed array mRNA assay, as described above.

Many of the above referenced techniques, as well as protocols for performing them, are described in Ausubel et al., eds., Current Protocols in Molecular Biology, 2002, Vol. 4, Unit 4, Ch. 6-9 and Unit 15, Ch. 5, which is hereby incorporated by reference in its entirety. For example, Northern blot assays, S1 nuclease protection assays, primer extension, RNAse protection assays, and reverse transcription PCR are well known to in the art and are each described in detail in this reference. One of ordinary skill in the art can readily adapt these protocols for detecting the presence and amount of an mRNA that is expressed by genes associated with hot flash symptoms.

Further, the presence and amount of an mRNA expressed from genes associated with hot flash symptoms can be accomplished using a dot blot format. In the dot blot format, the unlabeled amplified sample is bound to a solid support, such as a membrane, the membrane incubated with labeled probe under suitable hybridization conditions, the unhybridized probe removed by washing, and the filter monitored for the presence of bound probe. When multiple samples are analyzed with a single probe, the dot blot format is quite useful. Many samples can be immobilized at discrete locations on a single membrane and hybridized simultaneously by immersing the membrane in a solution of probe.

An alternate method that is quite useful when large numbers of different probes are to be used is a “reverse” dot blot format, in which the amplified sequence contains a label, and the probe is bound to the solid support. This format can be especially useful if the assay methods of the present invention is used as one of a battery of methods to be performed simultaneously on a sample. In this format, the unlabeled probes specific for a gene or several genes associated with hot flash symptoms are bound to the membrane and exposed to the labeled sample under appropriately stringent hybridization conditions. Unhybridized labeled sample is then removed by washing under suitably stringent conditions, and the filter is then monitored for the presence of bound sequences.

Both the forward and reverse dot blot assays can be carried out conveniently in a microtiter plate; see U.S. patent application Ser. No. 695,072, filed May 3, 1991, which is a CIP of U.S. patent application Ser. No. 414,542, filed Sep. 29, 1989, now abandoned, each of which is incorporated herein by reference in its entirety. The probes can be attached to bovine serum albumen (BSA), for example, which adheres to the microtiter plate, thereby immobilizing the probe. Another example of a method of using one or more oligonucleotides specific for genes associated with hot flash symptoms to detect the presence and amount of mRNA expressed from such genes is described in U.S. Pat. No. 6,383,756, which provides a method for detecting a nucleic acid bound to a membrane, and which is hereby incorporated by reference in its entirety.

In addition, Bustin, 2002, J. Mol. Endocrinol. 29(1):23-39; Bustin, 2000, J. Mol. Endocrinol.25(2):169-93; and Freeman et al., 1999, Biotechniques. 26(1):112-22, 124-5; each of which are incorporated by reference in their entirety, review the state of the art of real time reverse transcription PCR and provide methods for performing such reactions. These references also describe quantitative reverse transcription PCR, which is further reviewed by Richards and Poch, 2002, Mol. Biotechnol. 21(1):19-37, which is hereby incorporated by reference in its entirety. These references teach reaction conditions and parameters that can easily be adapted by one of skill in the art for detecting the presence and amount of an mRNA expressed from genes that are associated with hot flash symptoms.

5′-nuclease assays generally comprise contacting a primer hybridized to the nucleic acid to be detected with an enzyme with 5′-nuclease activity. The enzyme with 5′-nuclease activity then fragments a probe that is also hybridized to the nucleic acid to be detected in a 5′-nuclease reaction. The probe can be labeled with a detectable moiety that enables detection of fragmentation of the probe. Such methods are based on those described in U.S. Pat. Nos. 6,214,979, 5,804,375, 5,487,972 and 5,210,015, each of which is hereby incorporated by reference in its entirety. One of skill in the art is readily able to recognize suitable probes and primers for detecting a particular nucleic acid of known sequence for use in a 5′-nuclease assay for detecting the presence and amount of an mRNA expressed from genes associated with hot flash symptoms.

In another example, the presence and amount of mRNA expressed from genes associated with hot flash symptoms can be detected with a branched-DNA assay. In such methods, a dendrimer monomer is constructed of two DNA strands that share a region of sequence complementarity located in the central portion of each strand. When the two strands anneal to form the monomer the resulting structure has a central double-stranded center bordered by four single-stranded ends. A dendrimer can be assembled from monomers by hybridization of the single stranded ends of the monomers to each other, while still leaving many single-stranded ends free. These free single-stranded ends can have the sequences of any nucleic acid that can hybridize to an mRNA expressed from genes associated with hot flash symptoms. A dendrimer can be detectably labeled with any detectable moiety known to one of skill in the art without limitation, as described above in connection with the high throughput methods for quantifying an mRNA expressed from genes associated with hot flash symptoms.

Dendrimers can then be used as a probe, in, for example, the “dot blot” assays described above. In addition, a dendrimer can be used as a probe in any method known to one of skill in the art in which the probe is directly detected. A probe is directly detected when the presence of the probe can be determined without any subsequent reaction or modification, such as a dot blot or Northern hybridization. Further guidance on the selection and use of dendrimers as probes to detect the presence and amount of an mRNA expressed from genes associated with hot flash symptoms may be found in U.S. Pat. Nos. 6,261,779 and in Nilsen et al., 1997, J. Theoretical Biology 187:273-284, Capaldi et al., 2000, Nucleic. Acids Res., 28(7):21e, Wang et al., 1998, J. Am. Chem. Soc. 120:8281-8282, and Wang et al., 1998, Electroanalysis 10(8):553-556, each of which is hereby incorporated by reference in its entirety.

A reporter gene assay can also be used to detect the presence and amount of an mRNA expressed from genes associated with hot flash symptoms. Such assays generally rely on recombinantly linking the transcriptional control region of a gene associated with hot flash symptoms to the coding region of a reporter gene. The presence and amount of expression of the reporter gene can then be determined. The presence and amount of an mRNA expressed by genes associated with hot flash symptoms can then be extrapolated based upon the presence and amount of the reporter gene that is detected. Representative reporter gene assays that can easily be adapted by one of skill in the art for use in the methods of the invention are described by Storz et al., 1999, Anal. Biochem. 276:97-104 and Terstappen et al., 2000, J. Biomol. Screen. 5:255-262, each of which is incorporated by reference in its entirety.

The presence and amount of an mRNA expressed from genes associated with hot flash symptoms can also be determined using a bead array-based assay. In such assays, oligonucleotides specific for the mRNA to be detected are coupled to fluorescently-detectable beads. The mRNA is then reverse transcribed into labeled cDNA, and hybridized to the detectable beads. The presence and amount of the labeled cDNA can then be detected, revealing the presence and amount of the mRNA expressed from the gene associated with hot flash symptoms. One of ordinary skill in the art can readily adapt the protocols described in Yang et al., 2001, Genome Res. 11:1888-98 and in U.S. Pat. Nos. 6,562,569, 6,514,771, 6,468,811, 6,387,707, 6,376,256, 6,255,116, and 6,251,691, each of which is incorporated by reference in its entirety, to detect the presence and amount of an mRNA expressed from genes associated with hot flash symptoms.

Of course, the presence and amount of an mRNA expressed from genes associated with hot flash symptoms can be determined by reverse transcribing the mRNA into cDNA and detecting the presence and amount of cDNA product. Thus, any of the above-described methods suitable for the detection of DNA in addition to RNA can be adapted to detect cDNA derived from mRNA expressed from genes associated with hot flash symptoms.

Similarly, the presence and amount of mRNA expressed from genes associated with hot flash symptoms can also be determined by quantifying the amount of protein translated from the mRNA. As one of skill in the art is aware, the amount of protein translated from a given transcript, absent translational regulation, depends on the amount of mRNA transcript. Thus, the effects of candidate compounds on regulation of genes associated with hot flash symptoms can also be determined by detecting the presence and amount of proteins expressed from genes associated with hot flash symptoms.

The presence and amount of protein expressed from genes associated with hot flash symptoms can be determined by any suitable method known by one of skill in the art without limitation. For example, and not by way of limitation, the presence and amount of protein expressed from genes associated with hot flash symptoms can be determined using a western blot assay, an ELISA assay, a cytokine bead array, multiplexed protein detection assays, an immunofluorescence assay, and the like.

Many of the above-referenced techniques, as well as protocols for performing them, are described in Ausubel et al., eds., Current Protocols in Molecular Biology, 2002, Vol. 1, Unit 10, Ch. 3, and Vol. 2, Unit 11 Ch. 1-6., which is hereby incorporated by reference in its entirety. For example, western blot assays, ELISA assays, and immunofluorescence assays are well known to in the art and are each described in detail in this reference. One of ordinary skill in the art can readily adapt these protocols for detecting the presence and amount of protein that is expressed by genes associated with hot flash symptoms.

In addition, U.S. Pat. No. 6,576,478, which is incorporated by reference in its entirety, describes an array suitable for cytokine bead assays, multiplexed fluorescence assays, and other high throughput assays for determining the presence and amount of protein expressed from genes associated with hot flash symptoms. The device, as well as the device described in U.S. Pat. No. 6,558,960, also incorporated by reference in its entirety, can readily be adapted by one of skill in the art for use in such methods. U.S. Pat. Nos. 6,531,283 and 6,511,802, each of which is hereby incorporated by reference in its entirety, provide additional guidance regarding such assays for determining the presence and amount of protein expressed from genes associated with hot flash symptoms.

### 6.2.7. Primers and Probes Useful in Certain Methods for Quantifying the Expression of Genes Associated with Hot Flash Symptoms

The present invention further provides nucleic acid primers that are useful in certain methods for quantifying the expression of genes associated with hot flash symptoms. As one of skill in the art will readily recognize, certain of such methods described above rely on the use of oligonucleotide primers to amplify nucleic acids that comprise sequences of genes associated with hot flash symptoms. Methods of using such primers to reverse transcribe RNA and/or to amplify DNA of known sequence are well-known to the art and will not be presented in detail here. The primers, their sequences, and the genes from which sequences that the primers can amplify are derived are presented in TABLE 2, below.

TABLE 2

| | | |
|---|---|---|
| Gene | Primer 1 (Forward Primer) | Primer 2 (Reverse Primer) |

| | | |
|---|---|---|
| HKII | M68971 211F: | M6897 1378B: |
| | ATATGATCGCCTGCTTATTCA | AAAGGTAGGCAACATTTTCAC |
| | SEQ ID NO:28 | SEQ ID NO:29 |
| HKII | M68971 577F: | M68971 893B: |
| | TTGACCACATCGCCGAATGC | AGTGCCCACAATGAGACCAATC |
| | SEQ ID NO:30 | SEQ ID NO:31 |
| Parvalbumin | AI175539 250F: | AI175539 402B: |
| | CCCGTCCTTGTCTCCAG | AGAAAAAGAGTGCGGATGATG |
| | SEQ ID NO:32 | SEQ ID NO:33 |
| Parvalbumin | AI175539 41F: | AI175539 285B: |
| | AGCATTTTCCAGAAGAGTGGTGTC | ACAAAGACGCTGATGGCTGC |
| | SEQ ID NO:34 | SEQ ID NO:35 |
| Activin beta | AF089825 669F: | AF089825 918B: |
| E | GAGCACCAAACCACTTCCTC | CTACAACATAAGGGGGTCTC |
| | SEQ ID NO:36 | SEQ ID NO:37 |
| Activin beta | AF089825 492F: | AF089825 941B: |
| E | AAATCCACTTCAACCTACCGCTC | TCGTCTACAACATAAGGGGGTCTC |
| | SEQ ID NO:38 | SEQ ID NO:39 |
| Glutamate | U08255 642F: | U08525 945B: |
| receptor | TCTATGACAGCGAGTATGATA | CAAGGGCACTGTGGACCAGAT |
| | SEQ ID NO:40 | SEQ ID NO:41 |
| Glutamate | U08255 1030F: | U08255 1336B: |
| receptor | TAACCACCGCATCTCTTCCCTG | TGTGCCAAGGATTTCAAACTGG |
| | SEQ ID NO:42 | SEQ ID NO:43 |
| PKC epsilon | M18331 512F: | M18331 836B: |
| | CAGAATGGGAGCCGTCACTTC | AGCGCACTTCGTAATAATGAG |
| | SEQ ID NO:44 | SEQ ID NO:45 |
| PKC epsilon | M18331 1298F: | M18331 1631B: |
| | TTTGACAACCGAGGAGAGGAGC | CCTTGGTCTGGAAGCAGCAATAG |
| | SEQ ID NO:46 | SEQ ID NO:47 |
| EST 203549 | AI009098 115F: | AI009098 395B: |
| | GGGGAACTGTGTAGGACCTT | ATGTAAAAATGCCACCTCACT |
| | SEQ ID NO:48 | SEQ ID NO:49 |
| EST 203549 | AI009098 61F: | AI009098 382B: |
| | TTCAAACCTGTCCAACCAGCC | TTGTGGGTAAAGAAAGAGGGGTC |
| | SEQ ID NO:50 | SEQ ID NO:51 |
| VAP1 | AF034582 1239F: | AF034582 1573B: |
| | TCGCATCCGTGTCTACTCCATC | GGAAGTCCTTTTCTGTCACCACC |
| | SEQ ID NO:52 | SEQ ID NO:53 |
| VAP1 | AF034582 313F: | AF034582 655B: |
| | CAGAACCACCCCATTTACCTG | TGTTTACATCCAAGGCTCTCACTG |
| | SEQ ID NO:54 | SEQ ID NO:55 |
| MDR | M81855 2147F: | M81855 2468B: |
| | CGAAAGAGGATGTGGATGAAGATG | ATGTATCGGAGTCGCTTGGTGAGG |
| | SEQ ID NO:56 | SEQ ID NO:57 |
| MDR | M81855 2153F: | M81855 2468B: |
| | AGGATGTGGATGAAGATGTGCC | ATGTATCGGAGTCGCTTGGTGAGG |
| | SEQ ID NO:58 | SEQ ID NO:59 |
| Arginino- | X12459 448F: | X12459 766B: |
| succinate | GGAGGATGCCCGAGTTTTACAAC | AAGAGGTCCAAGGATGTGCTGTGG |
| synthetase | SEQ ID NO:60 | SEQ ID NO:61 |
| Arginino- | X12459 730F: | X12459 1057B: |
| succinate | AAGATGGCACTACCCACAGCAC | TTTCCTTCCACCCGTTCCTG |
| synthetase | SEQ ID NO:62 | SEQ ID NO:63 |
| BAD2 | U02553 865F: | U02553 1193B: |
| | ATCAAGGATGCTGGAGGAAGGG | TAGTTCAGGGCACTGTTCGTGG |
| | SEQ ID NO:64 | SEQ ID NO:65 |
| BAD2 | U02553 717F: | U02553 990B: |
| | CTTGGGTATCACTGCTTTGA | ATAATACTCCGCCTCTGCTTC |
| | SEQ ID NO:66 | SEQ ID NO:67 |
| Prolactin | AF022935 145F: | AF022935 528B: |
| | TGTTCTGGTGGCGACTGCCAGACACCT | TATCTTTTCAATCCCTTCAAGAAGCCG |
| | SEQ ID NO:68 | SEQ ID NO:69 |
| 2-alpha-1 | X56325 299F | X56325 562B: |
| globin | AAGAACTGCTGGGGGAAGATTG | TTGCCGTGAGCCTTGACCTG |
| | SEQ ID NO:70 | SEQ ID NO:71 |
| RNR1 | L08595 305F: | 640B: |
| | ACAACTACAGCACAGGCTACGACG | AGAAGAGTGAAAGGCGGGAGAC |
| | SEQ ID NO:72 | SEQ ID NO:73 |
| RNR1 | L08595 541F: | L08595 780B: |
| | GACGATCCGGGCTCCCTTCAC | ATGGATGCCGGCTTGCGAATG |
| | SEQ ID NO:74 | SEQ ID NO:75 |
| IL18 | AY077842 334F: | AY077842 546B: |
| | GACCACTTTGGCAGACTTCACTG | CCTTCCATCCTTCACAGATAGGG |
| | SEQ ID NO:76 | SEQ ID NO:77 |
| IL18 | AY077842 435F: | AY077842 709B: |
| | GCCTGATATCGACCGAACAGC | ATCATCTTCCTTTTGGCAAGC |
| | SEQ ID NO:78 | SEQ ID NO:79 |
| ARL gene 4 | X77235 135F: | X77235 410B: |
| | ACTTCCATCCTATCCAGCCTGC | CACCACAAACACAATGCCATCTG |
| | SEQ ID NO:80 | SEQ ID NO:81 |
| ARL gene 4 | X77235 301F: | X77235 617B: |
| | GCAATTCCAAAACAGTCAC | TTTAGCCCATCTCCTATGATT |
| | SEQ ID NO:82 | SEQ ID NO:83 |
| Calpain | Calpain 1424F: | Calpain 1791B: |
| | AGGCTACGCTGTCTACCAGATTCC | AACACCCTCAAGCAGAAGTCACC |
| | SEQ ID NO:84 | SEQ ID NO:85 |
| Calpain | Calpain 1224F: | Calpain 1356B: |
| | TGGACACGGGGTTCTACA | CAACTCCTTGGGAATCTGGTA |
| | SEQ ID NO:86 | SEQ ID NO:87 |
| EST 196325 | EST196325 131F: | EST196325 356B: |
| | GGAGCCATTGTTCACATTACCG | TACCCTGCCTTCTTCTCTCTGGAG |
| | SEQ ID NO:88 | SEQ ID NO:89 |
| EST 196325 | EST196325 150F: | EST196325 431B: |
| | CCGACCAGCAACACAGAGC | TTCGCCGTAAAACATCAGCAT |
| | SEQ ID NO:90 | SEQ ID NO:91 |
| CPP32 | CPP32 362F: | CPP32 630B: |
| | GGAGCAGTTTTGTGTGTGTGATTC | TGCGGTAGAGTAAGCATACAGGAAG |
| | SEQ ID NO:92 | SEQ ID NO:93 |
| CPP32 | CPP32 492F: | CPP32 796B: |
| | GCCGAAACTCTTCATCATTCA | GATCTGTTTCTTTGCGTGGAA |
| | SEQ ID NO:94 | SEQ ID NO:95 |
| Annexin1 | NM_012904 822F: | NM_012904 1041B: |
| | TGGAACTGAAGGGTGACATTGAG | ATGGCTTGGCAGAGAGGGATTC |
| | SEQ ID NO:96 | SEQ ID NO:97 |
| Annexin1 | NM_012904 824F: | NM_012904 1089B: |
| | GAACTGAAGGGTGACATTGAG | GGGATGTTTAGTTTCCTCCAC |
| | SEQ ID NO:98 | SEQ ID NO:99 |
| EST | AI014169 212F: | AI014169 393B: |
| AI014169 | CTGACACAGGACACGGAACAAAG | GGTGACACTCTTACATTGAGATGCC |
| | SEQ ID NO:100 | SEQ ID NO:101 |
| EST | AI014169 171F: | AI014169 360B: |
| AI014169 | CACAGTTCTCGGGTGGAGT | CATTGAGATGCCCTAACAGTG |
| | SEQ ID NO:102 | SEQ ID NO:103 |
| HBP1 | U09551 540F: | U09551 864B: |
| | GACCACTGGAAGGAAGAAACACC | CAGACTCACCGAATGACACACTCTC |
| | SEQ ID NO:104 | SEQ ID NO:105 |
| HBP1 | U09551 840F: | U09551 1131B: |
| | GAGAGTGTGTCATTCGGTGAGTCTG | CGGAAGAGTCCATAGGTGTGAAGTC |
| | SEQ ID NO:106 | SEQ ID NO:107 |
| Amiloride | X73911 819F: | X73911 1150B: |
| Binding | TGGCTCGGAAATACGCAGTTG | AGGTGTGTGTCCTCCATACAGTGC |
| Protein | SEQ ID NO:108 | SEQ ID NO:109 |
| Amiloride | X73911 1367F: | X73911 1687B: |
| Binding | GGTGGCTTCAACTTCTATGCGG | CCAGGGATTGGTGAGGTTTTCC |
| Protein | SEQ ID NO:110 | SEQ ID NO:111 |

Furthermore, the present invention provides probes that can also be used in certain methods for quantifying the expression of genes associated with hot flash symptoms. As one of skill in the art will readily recognize, certain of such methods described above rely on the use of nucleic acid probes to detect and quantify nucleic acids that comprise sequences of genes associated with hot flash symptoms. Methods of using such probes to detect and quantify nucleic acids of known sequence in, for example, RNAse protection assays, dot blots, and the like, are well-known to the art and will not be presented in detail here. The regions of genes associated with hot flash symptoms that can be used as probes to detect and quantify nucleic acids from such genes are presented in TABLE 3, below.

TABLE 3

| Gene Name | Sequence | Length of cDNA |
|---|---|---|
| MDR | 2147-2468 | 321 bp |
| Parvalbumin | 41-285 | 244 bp |
| HKII | 577-893 | 316 bp |
| BAD2 | 865-1193 | 294 bp |
| PKCε | 1298-1631 | 333 bp |
| IL18 | 334-546 | 213 bp |
| Calpain | 1224-1356 | 234 bp |
| EST196325 | 131-356 | 226 bp |
| Annexin1 | 822-1041 | 220 bp |
| EST207724(N27) | 212-393 | 180 bp |
| HBP1 | 840-1131 | 292 bp |
| PRL | 145-528 | 383 bp |

### 6.3. Methods of Identifying Candidate Compounds that Decrease the Incidence of Hot Flash Symptoms

In another aspect, the invention provides methods of identifying candidate compounds that decrease the incidence of hot flash symptoms. These methods generally comprise performing a method of the invention for determining the effect of a candidate compound on hot flash symptoms, and additionally determining that the candidate compound decreases the incidence of hot flash symptoms.

A candidate compound can be determined to decrease the incidence of hot flash symptoms based upon the effect of the candidate compound on regulation of genes associated with hot flash symptoms as extensively described above. In certain embodiments, the effect of the candidate compound on regulation of expression of genes associated with hot flash symptoms can be compared to a reference pattern of expression, as described above. In other embodiments, the effect of the candidate compound on regulation of expression of genes associated with hot flash symptoms can be compared to the effect of a compound with a known effect on regulation of expression of genes associated with hot flash symptoms, as described above.

### 6.4. Kits for Determining the Effect of a Candidate Compound on Hot Flash Symptoms

In another aspect, the invention provides kits for determining the effect of a compound on hot flash symptoms. The kits generally comprise at least one primer or probe that can be used to detect the presence and amount of an expression product of a member of a panel of genes associated with hot flash symptoms. The primer or probe can be any primer or probe known by one of skill in the art to be useful for detecting the presence and amount of an expression product of a member of a panel of genes associated with hot flash symptoms without limitation. The members of the panel of genes can be any genes associated with hot flash symptoms known by one of skill in the art without limitation, especially those described above.

In certain embodiments, the primer or probe can be used to detect an mRNA expressed from a member of a panel of genes associated with hot flash symptoms. Primers and probes suitable for such kits are extensively described in Section 5.2.7., above. In other embodiments, the primer or probe can be used to detect a protein expressed from a member of a panel of genes associated with hot flash symptoms.

In another aspect, the invention provides kits for determining the effect of one or more candidate compounds on hot flash symptoms. In certain embodiments, the kit comprises at least one gene-specific nuclease protection probe that is specific for a member of a panel of genes associated with hot flash symptoms; a surface having multiple spatially discrete regions, wherein at least two of the regions are substantially identical and wherein each region comprises at least two different oligonucleotide anchors; at least one bifunctional linker, wherein each bifunctional linker comprises a first region that can specifically bind to one of the oligonucleotide anchors and a second region that specifically binds to one of the gene-specific nuclease protection probe(s); and at least one detection probe, wherein the detection probe(s) specifically binds to the gene-specific nuclease protection probe(s).

In another embodiment, the kit for determining the effect of a compound on hot flash symptoms, comprises at least one gene-specific nuclease protection probe that is specific for a member of a panel of genes associated with hot flash symptoms and that can be directly or indirectly detectable; and a surface having multiple spatially discrete regions, at least two of which regions are substantially identical, and wherein the regions are adapted to specifically bind to the gene-specific protection probe(s).

The kits of the invention can comprise any components described as useful in the methods of the invention, above, without limitation. For example, gene-specific nuclease protection probes, detection probes, primers, and probes described as useful in the methods of the invention may also be included in a kit of the invention.

In certain embodiments, a kit of the invention can comprise instructions for determining the effect of a compound on hot flash symptoms. In certain embodiments, the kit can comprise instructions for identifying a compound that decreases the incidence of hot flash symptoms.

### 6.5. Compositions for Determining the Effect of a Candidate Compound on Hot Flash Symptoms

In yet another aspect, the invention provides compositions suitable for determining the effect of a compound on hot flash symptoms. The compositions may also be used in the methods and kits of the invention. In certain embodiments, the composition comprises a plurality of gene-specific nuclease protection probes, wherein each member of the plurality of gene-specific nuclease protection probes hybridizes under stringent conditions to an mRNA expressed from a member of a panel of genes associated with hot flash symptoms. The member of the panel of genes can be any member of a panel of genes associated with hot flash symptoms known by one of skill in the art without limitation.

In other embodiments, the composition comprises one or more primers that can be used to determine the presence and amount of an expression product of one or more genes associated with hot flash symptoms and a suitable buffer, diluent, or excipient. In certain embodiments, the primer or probe can be used to detect an mRNA expressed from a member of a panel of genes associated with hot flash symptoms. Primers and probes suitable for such kits are extensively described in Section 5.2.7., above. In other embodiments, the primer or probe can be used to detect a protein expressed from a member of a panel of genes associated with hot flash symptoms.

In certain embodiments, the member of the panel of genes associated with hot flash symptoms can be selected from the group consisting of Activin Beta E, Type II Hexokinase, Multi Drug Resistance Gene, Parvalbumin, BAD2, Prolactin, Argininosuccinate Synthetase, Ribonucleoside Reductase 1, Interleukin-18, ARL gene 4, Calpain, EST196325, CPP32, EST208064, 2-alpha-1 globin, Amiloride Binding Protein, Annexin 1, N27, HBP1, D-binding protein, FE65, Protein Kinase C type I, Glutamate Receptor subunit d1, VAP1, Protein Kinase C subspecies epsilon, EST203549, and Heat Shock Transcription Factor 1. In a preferred embodiment, the member of the panel of genes is selected from the group consisting of Type II Hexokinase, Multi Drug Resistance Gene, Parvalbumin, BAD2, Prolactin, Interleukin-18, Calpain, EST196325, Annexin 1, N27, HBP1, and Protein Kinase C subspecies epsilon.

### 6.6. Arrays for Determining the Effect of a Candidate Compound on Hot Flash Symptoms

In yet another aspect, the invention provides arrays useful for the identification of the effect of a plurality of compounds on hot flash symptoms. In certain embodiments, the array can comprise a non-porous surface; and a plurality of different oligonucleotides connected with the surface, wherein at least one of the oligonucleotides hybridizes under stringent conditions to a member of a panel of genes associated with hot flash symptoms, and wherein each of the different oligonucleotides is connected with the surface in a different predetermined region of the surface. The member of the panel of genes can be any member of a panel of genes associated with hot flash symptoms known by one of skill in the art without limitation.

In certain embodiments, the member of the panel of genes can be selected from the group consisting of Activin Beta E, Type II Hexokinase, Multi Drug Resistance Gene, Parvalbumin, BAD2, Prolactin, Argininosuccinate Synthetase, Ribonucleoside Reductase 1, Interleukin-18, ARL gene 4, Calpain, EST196325, CPP32, EST208064, 2-alpha-1 globin, Amiloride Binding Protein, Annexin 1, N27, HBP1, D-binding protein, FE65, Protein Kinase C type I, Glutamate Receptor subunit d1, VAP1, Protein Kinase C subspecies epsilon, EST203549, and Heat Shock Transcription Factor 1. In a preferred embodiment, the member of the panel of genes is selected from the group consisting of Type II Hexokinase, Multi Drug Resistance Gene, Parvalbumin, BAD2, Prolactin, Interleukin-18, Calpain, EST196325, Annexin 1, N27, HBP1, and Protein Kinase C subspecies epsilon.

### 7. EXAMPLES

The invention is described in reference to a number of examples presented below. The examples are intended to provide illustration of certain embodiments of the invention, and should not be construed to limit the invention in any way.

### 7.1. Example 1

### Generation of Reference Expression Profiles of a Panel of Genes Associated with Hot Flash Symptoms

The following example describes the generation of expression profiles of a panel of genes whose expression level is associated with the increase or decrease of hot flash symptoms using the compounds estradiol, raloxifene, tibolone, and 4-hydroxy tamoxifen. Estradiol and raloxifene are known to have a decreasing effect on hot flash symptoms, while tibolone and 4-hydroxy tamoxifen are known to have an increasing effect on hot flash symptoms.

Treatment of GH3 Cells and Preparation of Cell Lysates. In 96-well V-bottom dishes, 50,000 GH3 cells per well were plated in 200 μl phenol red free Ham's F-12K media supplemented with 2.5% charcoal stripped FCS, 1% penicillin & streptomycin, 1% 200 mM glutamine. The plates was incubated at 37° C. for 24 hours in a humidified atmosphere containing 5% (v/v) CO₂.

The next day, compound dilutions were prepared according to a standard compound dilution protocol: Briefly, from a 5 mM stock, the compounds (estradiol, raloxifene, tibolone, and 4-hydroxy tamoxifen ) were serially diluted in 100% DMSO from 5 mM to 0.00005 mM. Next, the compounds were diluted from 100% DMSO to into water by 1:25 to reduce the DMSO concentration to 4% by transferring 10 μl of compound solution into 240 μl of water. After mixing the compound solutions well, 10 μl of diluted compounds were added to each well of the 96-well plates. As such, the cells were exposed to a final compound concentration of 10 μM to 0.0001 μM. The 96-well plates were then incubated at 37° C. for 24 hours.

The lysis buffer containing probe linker solution and the denaturation oil were warmed to 50° C. The plate heat block/oven was set to 95° C.

The plates were removed from the incubator and spun down at 1200 rpm for 5 minutes to ensure that the cells will not lift off. The media was aspirated from the wells using the 8-channel aspirator, and immediately thereafter, 30 μl of lysis buffer/probe-linker solution per well were added, followed by 60 μl of denaturation oil per well. The plates were covered with foil plate cover and sealed. Then the plates were heated at 95° C. for 15 minutes.

Analysis of Gene Expression. The panel of signature genes used in this example included protein tyrosine phosphatase BAD2, Type II Hexokinase, multiple drug resistance gene, parvalbumin, prolactin, IL-18, N27, calpain, annexin I, and PKC subunit epsilon. Expression of the listed genes along with four housekeeping genes, i.e., actin, GAPDH, cyclophilin and L32, were analyzed using a cell lysate nuclease protection assay kit from High Throughput Genomics, Tucson, Ariz., following the manufacturer's protocol. Custom-made detection probes and linkers required to perform the assay had been obtained from the manufacturer.

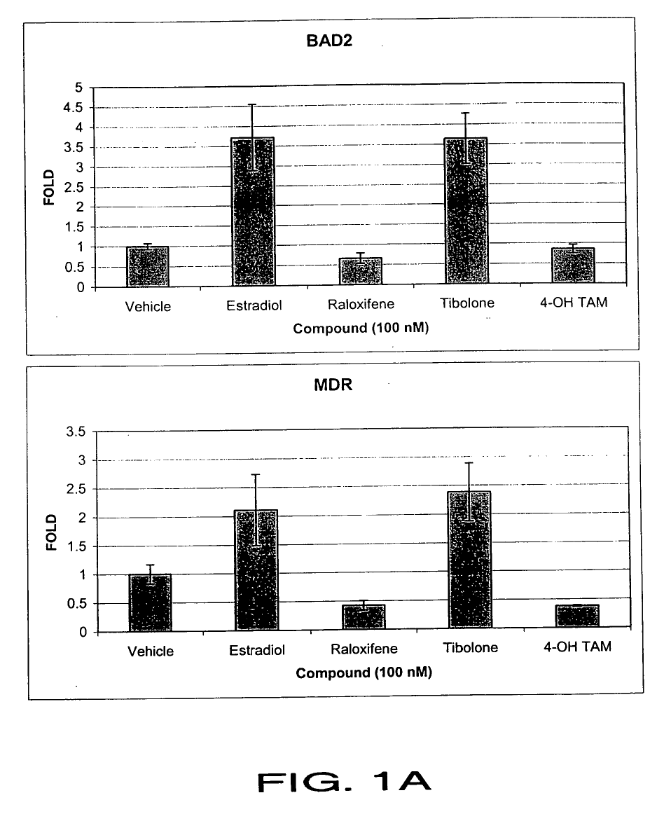

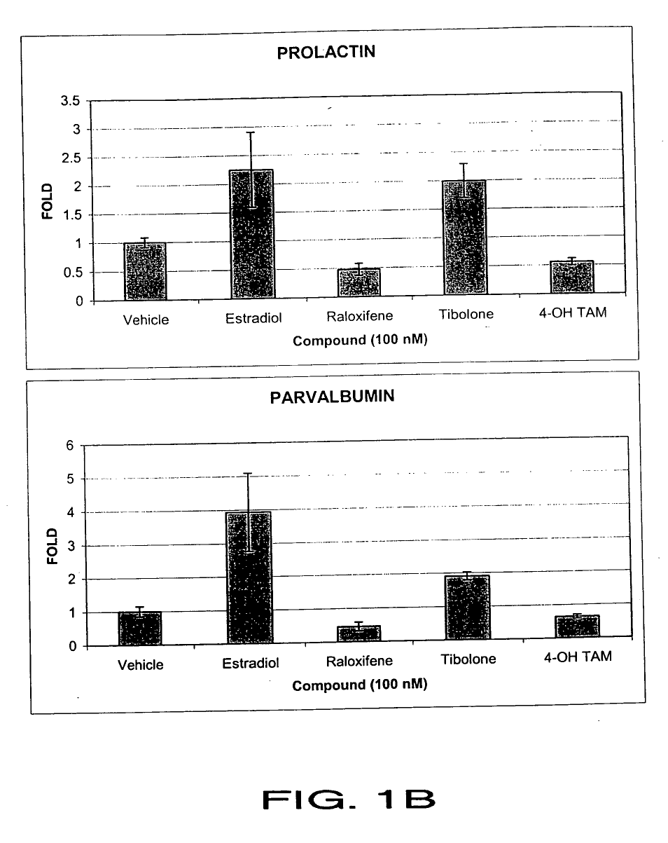

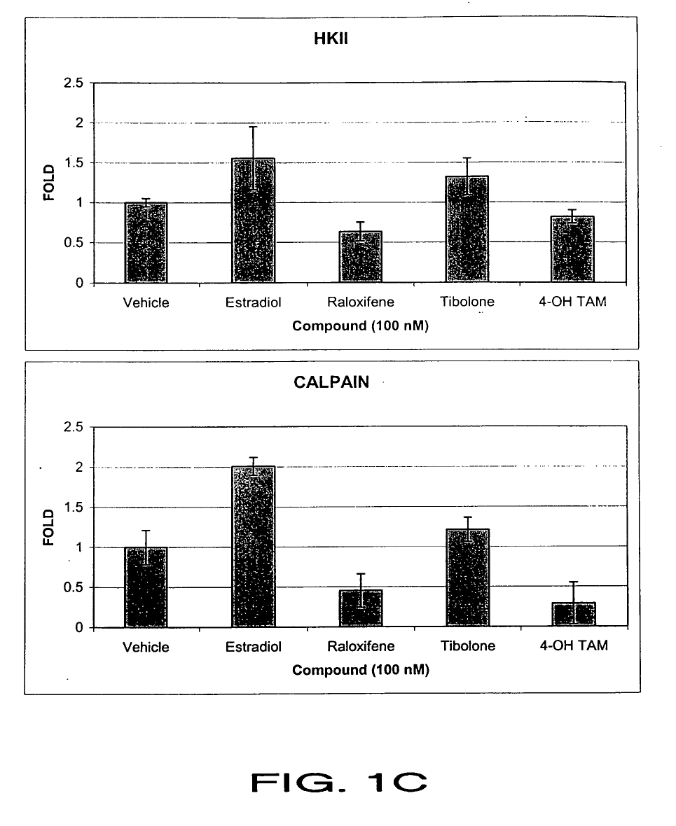

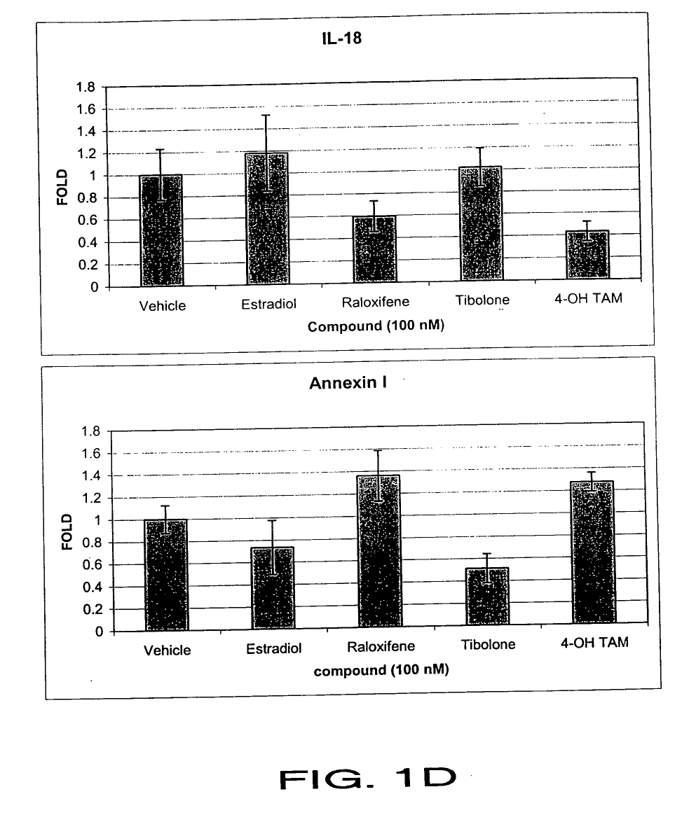

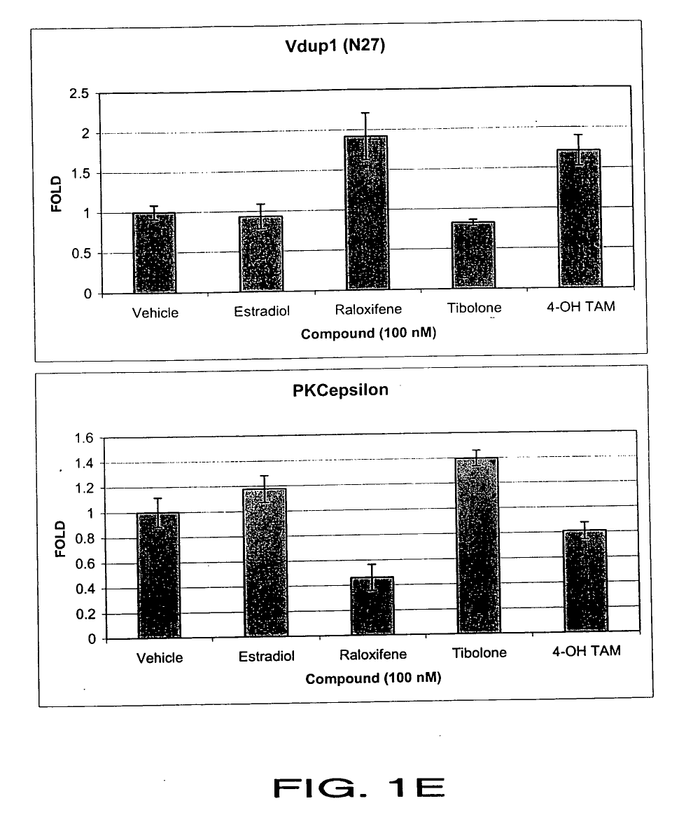

Data Analysis. First, gene expression data were normalized using the housekeeping gene expression. Then, the expression level of each gene in the cell samples treated with estradiol, raloxifene, tibolone, and 4-hydroxy tamoxifen was divided by the corresponding gene expression level in DMSO treated control samples. The effect of the compounds estradiol, raloxifene, tibolone, and 4-hydroxy tamoxifen, respectively, on the expression of each signature gene is shown in FIGS. 1A through 1E. These expression profiles serve as reference expression profiles for the testing of candidate compounds.

### 7.2. Example 2

### High Throughput Assay for Determining the Effect Candidate Compounds on Hot Flash Symptoms

The following example describes a high throughput assay for determining the effect of candidate compounds on the expression of a panel of genes whose expression level is associated with increase or decrease of hot flash symptoms. The rat pituitary cell line GH3 is used as the model system. Estradiol and Raloxifene at 100 nM are included in each experiment as reference compounds.

Treatment of GH3 Cells and Preparation of Cell Lysates. In 96-well V-bottom dishes, 50,000 GH3 cells per well are plated in 200 μl phenol red free Ham's F-12K media supplemented with 2.5% charcoal stripped FCS, 1% penicillin & streptomycin, 1% 200 mM glutamine. The plates are incubated at 37° C. for 24 hours in a humidified atmosphere containing 5% (v/v) CO₂.

The next day, candidate compound dilutions are prepared according to the compound dilution protocol described in Example 1, such that the cells are exposed to a final compound concentration of 10 μM to 0.0001 μM. The 96-well plates are then incubated at 37° C. for 24 hours.

As described in Example 1, the lysis buffer containing probe linker solution and the denaturation oil is warmed to 50° C. The plates are removed from the incubator and spun down at 1200 rpm for 5 minutes to ensure that the cells will not lift off. The media is aspirated from the wells using the 8-channel aspirator, and immediately thereafter, 30 μl of lysis buffer/probe-linker solution per well is added, followed by 60 μl of denaturation oil per well. The plates are covered with foil plate cover and sealed, and the plates are heated at 95° C. for 15 minutes.

Analysis of Gene Expression. Expression levels of the signature genes listed in Example 1, along with the expression levels of the four housekeeping genes used in Example 1, are analyzed using the cell lysate nuclease protection assay kit from High Throughput Genomics, Tucson, Ariz., following the manufacturer's protocol, and detection probes that had been custom-ordered from the manufacturer.

Data Analysis. After normalizing the gene expression data using the housekeeping gene expression, the expression level of each gene in the cell samples treated with candidate compounds is divided by the corresponding gene expression level in DMSO treated control samples. The effect of each candidate compound on the expression level of the panel of signature genes is compared with the reference profiles shown in FIGS. 1A through 1E.

### 7.3. Example 3

### Assay for Determining Hot Flash Side Effects of an Osteoporosis Candidate Compound

The assays of the present invention may be used to screen compounds identified as potential therapeutic agents for the treatment of osteoporosis (or other medical conditions) for hot flash side effects prior to costly clinical trials. The compound is assayed as described in Example 1. The effect of the compound on the expression of the panel of genes listed in Example 1 is compared with the expression profiles of estradiol, tibolone, raloxifene and 4-hydroxy tamoxifen. A compound whose effect on the expression profile of the panel of signature genes mirrors more closely that of raloxifene and 4-hydroxy tamoxifen than that of estradiol and tibolone is likely to increase the incidence of undesirable hot flash side effects.

### 7.4. Example 4

### Validation of Candidate Compounds Using a Rat Model of Hot Flash Symptoms

The following animal model is used to validate the effects of a candidate compound on hot flash symptoms.

Ovariectomized rats are treated for 8 or 9 days with candidate compounds. Rats are made morphine-dependent by implanting a morphine pellet (75 mg each) subcutaneously (sc) on days 3 and 5 of treatment. On the last day of treatment, a thermistor, connected to a data acquisition system, is placed on the tail of each animal and morphine addiction is withdrawn by naloxone injection (1.0 mg/kg, sc). Temperature measurements are taken for 1 h under ketamine (80 mg/kg, im) anesthesia. The candidate compounds that decrease the incidence of hot flash symptoms decrease the temperature of the rat tail in this model.

1. A method for determining the effect of a candidate compound on hot-flash symptoms, comprising:
a) contacting a first cell that expresses an estrogen receptor or estrogen related receptor with said candidate compound; and
b) determining the effect of said candidate compound on said first cell's expression of a panel of genes associated with hot flash symptoms.

2. The method of claim 1, wherein said method further comprises comparing said first cell's expression of said panel of genes associated with hot flash symptoms with a reference expression profile of said panel of genes associated with hot flash symptoms.

3. The method of claim 2, wherein said reference expression profile of said panel of genes is the expression profile of the panel of genes following contacting the cell with a compound selected from the group consisting of estradiol, tibolone, raloxifene, and tamoxifen.

4. The method of claim 1, wherein said method further comprises comparing said first cell's expression of said panel of genes associated with hot flash symptoms with a second cell's expression of said panel of genes associated with hot flash symptoms following contact with a compound that has a known effect on hot flash symptoms.

5. The method of claim 4, wherein said compound that has a known effect on hot flash symptoms is selected from the group that consists of estradiol, tibolone, raloxifene, and tamoxifen.

6. The method of claim 2 or 4, wherein said method comprises determining that said compound decreases the incidence of hot flash symptoms.

7. The method of claim 1, wherein said estrogen receptor is estrogen receptor α.

8. The method of claim 1, wherein said estrogen receptor is estrogen receptor β.

9. The method of claim 1, wherein said cell expresses both estrogen receptor α and estrogen receptor β.

10. The method of claim 1, wherein said cell that expresses the estrogen receptor is selected from the group consisting of a pituitary cell and a hypothalamus cell.

11. The method of claim 10, wherein said cell that expresses the estrogen receptor is selected from the group consisting of a GH3 cell, a GH4 cell, a PR1 cell, a MtT/E-2 cell, an alphaT3-1 cell, a D12 cell, an RCF-8 cell, and a GT1-7 cell.

12. The method of claim 1, wherein said cell's expression of said panel of genes associated with hot flash symptoms is quantified by determining the presence and amount of mRNA expressed from said panel of genes.

13. The method of claim 12 wherein said cell's expression of said panel of genes associated with hot flash symptoms is quantified by a technique selected from the group of reverse transcription real time PCR, quantitative reverse transcription PCR, Northern blot assays, dot blot assays, reverse dot blot assays, RNAse protection assays, 5′-nuclease assays, reporter gene assays, branched DNA assays, bead array assays, and multiplexed array mRNA assays.

14. The method of claim 13, wherein said cell's expression of said panel of genes associated with hot flash symptoms is quantified by a multiplexed array mRNA assay.

15. The method of claim 1, wherein said cell's expression of said panel of genes associated with hot flash symptoms is quantified by determining the presence and amount of protein expressed from said panel of genes.

16. The method of claim 15, wherein said cell's expression of said panel of genes associated with hot flash symptoms is quantified by a technique selected from the group of a western blot assay, an ELISA assay, a cytokine bead array, multiplexed protein detection assays, and an immunofluorescence assay.

17. The method of claim 1, wherein at least one member of said panel of genes is selected from the group consisting of Activin Beta E, Type II Hexokinase, Multi Drug Resistance Gene, Parvalbumin, BAD2, Prolactin, Argininosuccinate Synthetase, Ribonucleoside Reductase 1, Interleukin-18, ARL gene 4, Calpain, EST196325, CPP32, EST208064, 2-alpha-1 globin, Amiloride Binding Protein, Annexin 1, N27, HBP1, D-binding protein, FE65, Protein Kinase C type I, Glutamate Receptor subunit d1, VAP1, Protein Kinase C subspecies epsilon, EST203549, and Heat Shock Transcription Factor 1.

18. The method of claim 17, wherein at least one member of said panel of genes is selected from the group consisting of Type II Hexokinase, Multi Drug Resistance Gene, Parvalbumin, BAD2, Interleukin-18, Calpain, EST196325, Annexin 1, N27, HBP1, and Protein Kinase C subspecies epsilon, and wherein expression of at least one member of said panel of genes is upregulated in said cell following said contact with said candidate compound.

19. The method of claim 17, wherein at least one member of said panel of genes is selected from the group consisting of Type II Hexokinase, Multi Drug Resistance Gene, Parvalbumin, BAD2, Interleukin-18, Calpain, EST196325, Annexin 1, N27, HBP1, and Protein Kinase C subspecies epsilon, and wherein expression of at least one member of said panel of genes is not upregulated in said cell following said contact with said candidate compound.

20. The method of claim 17, wherein at least one member of said panel of genes is selected from the group consisting of Type II Hexokinase, Multi Drug Resistance Gene, Parvalbumin, BAD2, Prolactin, Interleukin-18, Calpain, EST196325, Annexin 1, N27, HBP1, and Protein Kinase C subspecies epsilon, and wherein expression of at least one member of said panel of genes is downregulated in said cell following said contact with said candidate compound.

21. The method of claim 17, wherein at least one member of said panel of genes is selected from the group consisting of Type II Hexokinase, Multi Drug Resistance Gene, Parvalbumin, BAD2, Prolactin, Interleukin-18, Calpain, EST196325, Annexin 1, N27, HBP1, and Protein Kinase C subspecies epsilon, and wherein expression of at least one member of said panel of genes is not downregulated in said cell following said contact with said candidate compound.

22. The method of claim 6, wherein said panel of genes comprises Type II Hexokinase, Multi Drug Resistance Gene, Parvalbumin, BAD2, Prolactin, Interleukin-18, Calpain, EST196325, Annexin 1, N27, and HBP1; wherein expression of Type II Hexokinase, Multi Drug Resistance Gene, Parvalbumin, BAD2, Prolactin, Interleukin-18, Calpain, and EST196325 is upregulated in said cell following said contact with said candidate compound; wherein expression of Annexin 1, N27, and HBP1 is not upregulated in said cell following said contact with said candidate compound; and wherein expression of Protein Kinase C subspecies epsilon is not downregulated in said cell following said contact with said candidate compound.

23. A method for rapidly determining the effects of a plurality of compounds on hot-flash symptoms, comprising:
a) separately contacting a sample of cells that express an estrogen receptor or estrogen related receptor with each member of said plurality of compounds; and
b) assessing the effect of each member of said plurality of compounds on each of said samples of cells' expression of a panel of genes associated with hot flash symptoms, thereby predicting the effect of each of said compounds on hot-flash symptoms.

24. The method of claim 23, wherein said method further comprises comparing said expression of said panel of genes associated with hot flash symptoms by said samples of cells with a reference expression profile of said panel of genes associated with hot flash symptoms.

25. The method of claim 24, wherein said reference expression profile of said panel of genes is the expression profile of the panel of genes following contacting the cell with a compound selected from the group consisting of estradiol, tibolone, raloxifene, and tamoxifen.

26. The method of claim 23, wherein said method further comprises comparing said expression of said panel of genes associated with hot flash symptoms by said samples of cells with the expression of said panel of genes associated with hot flash symptoms by a sample of cells following contact with a compound that has a known effect on hot flash symptoms.

27. The method of claim 26, wherein said compound that has a known effect on hot flash symptoms is selected from the group that consists of estradiol, tibolone, raloxifene, and tamoxifen.

28. The method of claim 24 or 26, wherein said method comprises determining that said compound decreases the incidence of hot flash symptoms.

29. The method of claim 23, wherein said estrogen receptor is estrogen receptor α.

30. The method of claim 23, wherein said estrogen receptor is estrogen receptor β.

31. The method of claim 23, wherein said sample of cells expresses both estrogen receptor α and estrogen receptor β.

32. The method of claim 23, wherein said sample of cells that expresses the estrogen receptor is selected from the group consisting of a pituitary cell and a hypothalamus cell.

33. The method of claim 32, wherein said sample of cells that expresses the estrogen receptor is selected from the group consisting of a GH3 cell, a GH4 cell, a PR1 cell, a MtT/E-2 cell, a alphaT3-1 cell, a D12 cell, an RCF-8 cell, and a GT1-7 cell.

34. The method of claim 23, wherein said expression of said panel of genes associated with hot flash symptoms by said sample of cells is quantified by determining the presence and amount of mRNA expressed from said panel of genes.

35. The method of claim 34, wherein said expression of said panel of genes associated with hot flash symptoms by said sample of cells is quantified by a technique selected from the group of reverse transcription real time PCR, quantitative reverse transcription PCR, Northern blot assays, dot blot assays, reverse dot blot assays, RNAse protection assays, 5′-nuclease assays, reporter gene assays, branched DNA assays, bead array assays, and multiplexed array mRNA assays.

36. The method of claim 35, wherein said expression of said panel of genes associated with hot flash symptoms by said sample of cells is quantified by a multiplexed array mRNA assay.

37. The method of claim 23, wherein said expression of said panel of genes associated with hot flash symptoms by said sample of cells is quantified by determining the presence and amount of protein expressed from said panel of genes.

38. The method of claim 37, wherein said expression of said panel of genes associated with hot flash symptoms by said sample of cells is quantified by a technique selected from the group of a western blot assay, an ELISA assay, a cytokine bead array, multiplexed protein detection assays, and an immunofluorescence assay.

39. The method of claim 23, wherein at least one member of said panel of genes is selected from the group consisting of Activin Beta E, Type II Hexokinase, Multi Drug Resistance Gene, Parvalbumin, BAD2, Prolactin, Argininosuccinate Synthetase, Ribonucleoside Reductase 1, Interleukin-18, ARL gene 4, Calpain, EST196325, CPP32, EST208064, 2-alpha-1 globin, Amiloride Binding Protein, Annexin 1, N27, HBP1, D-binding protein, FE65, Protein Kinase C type I, Glutamate Receptor subunit d1, VAP1, Protein Kinase C subspecies epsilon, EST203549, and Heat Shock Transcription Factor 1.

40. The method of claim 39, wherein at least one member of said panel of genes is selected from the group consisting of Type II Hexokinase, Multi Drug Resistance Gene, Parvalbumin, BAD2, Interleukin-18, Calpain, EST196325, Annexin 1, N27, HBP1, and Protein Kinase C subspecies epsilon, and wherein expression of at least one member of said panel of genes is upregulated in said sample of cells following said contact with said candidate compound.

41. The method of claim 39, wherein at least one member of said panel of genes is selected from the group consisting of Type II Hexokinase, Multi Drug Resistance Gene, Parvalbumin, BAD2, Interleukin-18, Calpain, EST196325, Annexin 1, N27, HBP1, and Protein Kinase C subspecies epsilon, and wherein expression of at least one member of said panel of genes is not upregulated in said sample of cells following said contact with said candidate compound.

42. The method of claim 39, wherein at least one member of said panel of genes is selected from the group consisting of Type II Hexokinase, Multi Drug Resistance Gene, Parvalbumin, BAD2, Prolactin, Interleukin-18, Calpain, EST196325, Annexin 1, N27, HBP1, and Protein Kinase C subspecies epsilon, and wherein expression of at least one member of said panel of genes is downregulated in said sample of cells following said contact with said candidate compound.

43. The method of claim 39, wherein at least one member of said panel of genes is selected from the group consisting of Type II Hexokinase, Multi Drug Resistance Gene, Parvalbumin, BAD2, Prolactin, Interleukin-18, Calpain, EST196325, Annexin 1, N27, HBP1, and Protein Kinase C subspecies epsilon, and wherein expression of at least one member of said panel of genes is not downregulated in said sample of cells following said contact with said candidate compound.

44. The method of claim 28, wherein said panel of genes comprises Type II Hexokinase, Multi Drug Resistance Gene, Parvalbumin, BAD2, Prolactin, Interleukin-18, Calpain, EST196325, Annexin 1, N27, and HBP1; wherein expression of Type II Hexokinase, Multi Drug Resistance Gene, Parvalbumin, BAD2, Prolactin, Interleukin-18, Calpain, and EST196325 is upregulated in said sample of cells following said contact with said candidate compound; wherein expression of Annexin 1, N27, and HBP1 is not upregulated in said sample of cells following said contact with said candidate compound; and wherein expression of Protein Kinase C subspecies epsilon is not downregulated in said sample of cells following said contact with said candidate compound.

45. An array, comprising:
a) a non-porous surface; and
b) a plurality of different oligonucleotides connected with said surface, wherein at least one of said oligonucleotides is specific for a member of a panel of genes associated with hot flash symptoms, and wherein each of said different oligonucleotides is connected with said surface in a different predetermined region of said surface.

46. The array of claim 45, wherein at least one of said oligonucleotides hybridizes under stringent conditions to a member of a panel of genes associated with hot flash symptoms.

47. The array of claim 46, wherein said member of said panel of genes is selected from the group consisting of Activin Beta E, Type II Hexokinase, Multi Drug Resistance Gene, Parvalbumin, BAD2, Prolactin, Argininosuccinate Synthetase, Ribonucleoside Reductase 1, Interleukin-18, ARL gene 4, Calpain, EST196325, CPP32, EST208064, 2-alpha-1 globin, Amiloride Binding Protein, Annexin 1, N27, HBP1, D-binding protein, FE65, Protein Kinase C type I, Glutamate Receptor subunit d1, VAP1, Protein Kinase C subspecies epsilon, EST203549, and Heat Shock Transcription Factor 1.

48. The array of claim 47, wherein said member of said panel of genes is selected from the group consisting of Type II Hexokinase, Multi Drug Resistance Gene, Parvalbumin, BAD2, Prolactin, Interleukin-18, Calpain, EST196325, Annexin 1, N27, HBP1, and Protein Kinase C subspecies epsilon.
